(19) United States
(12) Patent Application Publication
(10) Pub. No.: US 2018/0235984 A1
(43) Pub. Date: Aug. 23, 2018

(54) TETRACYCLINE MANAGEMENT OF EGFR INHIBITOR ASSOCIATED DERMATOSES

(71) Applicant: Foamix Pharmaceuticals Ltd., Rehovot (IL)

(72) Inventors: Meir EINI, Ness Ziona (IL); Mitchell SHIRVAN, Kfar Saba (IL); Rita KEYNAN, Rehovot (IL); Tal BERMAN, Rishon Le Ziyyon (IL); David SCHUZ, Gimzu (IL); Yohan HAZOT, Rehovot (IL)

(73) Assignee: Foamix Pharmaceuticals Ltd., Rehovot (IL)

(21) Appl. No.: 15/753,508

(22) PCT Filed: Aug. 19, 2016
(86) PCT No.: PCT/IB2016/054989

§ 371 Date: Feb. 19, 2018

Related U.S. Application Data
(60) Provisional application No. 62/345,695, filed on Jun. 3, 2016.
(60) Provisional application No. 62/248,008, filed on Oct. 29, 2015.
(60) Provisional application No. 62/207,712, filed on Aug. 20, 2015.

(51) Int. Cl. A61K 31/65; A61K 9/00; A61K 47/44; A61K 47/24; A61K 47/10; A61K 47/12; A61P 17/10

(52) U.S. Cl. CPC A61K 31/65; A61K 9/0014; A61K 47/44; A61P 17/10; A61K 47/10; A61K 47/12; A61K 47/24

(57) ABSTRACT

Methods of treatment and dosage regimes using a composition comprising a tetracycline antibiotic in treating or alleviating a disorder including EGFRI associated rash, EGFRI associated rash related symptoms, a tetracycline antibiotic responsive EGFRI associated rash related disorder, skin disorder caused by a bacteria, and a tetracycline antibiotic responsive sebaceous gland disease, P. EGFRI associated rash bacteria associated disorders and other superficial infections, including skin infections are provided.

### CROSS-REFERENCE TO RELATED APPLICATIONS

This application claims priority to U.S. Provisional Application Ser. No. 62/207,712, filed Aug. 20, 2015; 62/248,008, filed Oct. 29, 2015; and 62/345,695, filed Jun. 3, 2016, all of which are incorporated by reference in their entirety.

### BACKGROUND

Epidermal growth factor receptor (EGFR) is often overexpressed or dysregulated in a variety of solid tumours, including gastrointestinal (GI) malignancies. Since EGFR plays a central role in tumour growth, survival, proliferation, angiogenesis, invasiveness and metastatic spread, and since over-expression of EGFR is linked with disease progression, reduced survival, poor response to treatment, and resistance to anti-tumor treatments, specific targeting of the EGFR-mediated signalling pathway has become an increasing part of the therapeutic strategy in the treatment of advanced lung, head-and neck, and colorectal carcinoma (CRC).

An important class of drugs currently used in cancer therapy are known as epidermal growth factor receptor inhibitors (EGFRIs) which include monoclonal antibodies (mAb) that target the extracellular ligand-binding domain of EGFR, such as cetuximab, panitumumab, necitumumab, zalutumumab, mAb 806, mAb ICR63, mAb ICR80, mAb 225, nimotuzumab, and matuzumab, as well as tyrosine kinase inhibitors that block the intracellular tyrosine kinase (TK) domain, such as erlotinib, gefitinib, lapatinib, afatinib, imatinib, nilotinib, bosutinib, ponatinib, Bcr-Abl tyrosine kinase inhibitor, sunitinib, dasatinib, canertinib or vandetanib. Unlike standard chemotherapy, which affects most replicating cells, EGFRIs specifically target pathways that impact cancer cell growth and survival. Therefore, treatment with EGFRIs is well tolerated, and associated with a decreased incidence of systemic side effects in comparison with standard chemotherapeutic drugs.

Despite these benefits, the increasing clinical use of EGFRIs and the corresponding surveillance of patients have led to the identification of a range of EGFRI-specific side effects, which can result in decreased quality of life as well as a decrease, interruption or discontinuation of EGFRI treatment. These reactions are most evident in tissues that are dependent on EGFR signalling for normal function, such as the skin, nails, and mucosal membranes. The most commonly reported side effect is a distinct papulopustular rash, characterized by red papulopustules, which occurs in up to 90 percent of patients treated with EGFR inhibitors. For example, 75% of patients treated with Erlotinib 150 mg QD developed a rash in all grades, and 9% of the patients developed a grade 3 rash; 85% of patients treated with Cetuxima developed a rash in all grades, and 10% of the patients developed a grade 3 rash; and 90% of patients treated with panitumumab developed a rash in all grades and 16% of the patients developed a grade 3 rash.

The onset of the papulopustular rash is most commonly observed during the first week to second week of treatment with an EGFR inhibitor, although the range of onset reported in the literature is between two days and six weeks. The rash typically progresses through four phases: phase one (weeks 0-1) begins with sensory disturbances with erythema and edema, phase two (weeks 1-3) involves eruptions of the papulopustular lesions, phase three (weeks 3-5) advances to crusting of these eruptions, and phase four (weeks 5-8) is characterized by persistent dry skin, erythema, and telangiectasias. The incidence and severity of the EGFRI-associated rashiform-like eruption differs between classes of EGFR inhibitors. In general, skin rash associated with the use of EGFR-targeted monoclonal antibodies tends to be more severe and to occur with higher incidence than is observed with tyrosine kinase inhibitors. See, e.g., Busam K J, et al. Br J Dermatol. 2001; 144:1169; Mario E. Lacouture et al. Support Care Cancer (2010) 18(4):509-22; Lacouture M E and Lai S E. Br J Dermatol. 2006; 155:852-4; and B. Melosky et al. Current Oncology (2009) 16(1):16-26.

Although this rash is typically mild or moderate in severity, it can cause significant physical and psychosocial distress in patients, leading to decreased quality of life, and discontinuation or disruption of therapy. Surprisingly, a correlation was established between rash severity and the ability of the antibody to improve survival in CRC. In fact, the rash may serve as a surrogate marker of EGFRI-targeted mAb efficacy. Consequently, 76% of clinicians reported holding or pausing EGFRI treatment at some point during therapy due to the skin rash, and up to 32% of physicians discontinued EGFRI treatment altogether. Thus, ironically, the patients most likely to benefit from the EGFRI treatment are the ones who are most likely to limit or even discontinue treatment due to pain, itching, discomfort and social and emotional anxiety related to the side effects of this treatment. See, e.g., B. Melosky et al. Current Oncology (2009) 16(1):16-26.

Terms such as “EGFRI associated acne-like rash” or “EGFRI associated acneiform rash”, are sometimes used to describe this unique rash due to the appearance of lesions. However, EGFRI-induced skin toxicities do not present with comedones, and lesions are frequently itchy, respond to anti-inflammatory drugs and not to anti-acne agents, and, unlike acne, might affect areas such as the lower legs and dorsal arms. EGFRI associated rashiform eruption could also be seen in the seborrheic areas of skin including the face, scalp, neck, posterior auricular area, shoulders, and chest. An expert panel of oncologists and dermatologists familiar with anti-EGFR therapy recently suggested that this reaction may represent an entirely new dermatologic entity. See, e.g., Lacouture M E and Lai S E. Br J Dermatol. 2006; 155:852-4; and B. Melosky et al. Current Oncology (2009) 16(1): 16-26.

Other side effects of EGFR inhibitors include dry skin, pruritus, fissures, palmar-plantar rash, hyperkeratosis, telangiectasia, hyperpigmentation, blisters, mucositis, and pyogenic granuloma. Changes may also occur to the hair (for example, alopecia of the scalp or trichomegaly of the eyelashes) and nails (usually periungual manifestations such as paronychia).

Current management of EGFR inhibitor associated rash includes oral tetracyclines (minocycline and doxycycline), oral isotretinoin, oral steroids and antihistamines. See, e.g., Scope, A. L. C. Agero, S. W. Dusza et al., Journal of Clinical Oncology, vol. 25, no. 34, pp. 5390-5396, 2007; A. Jatoi, K. Rowland, J. A. Sloan et al., Cancer, vol. 113, no. 4, pp. 847-853, 2008; M. E. Lacouture, E. P. Mitchell, B. Piperdi et al., Journal of Clinical Oncology, vol. 28, no. 8, pp. 1351-1357, 2010; G. Deplanque, J. Chavaillon, A. Vergnenegre et al., Journal of Clinical Oncology, vol. 28, abstract 9019, 2010; R. Gutzmer, T. Werfel, R. Mao, A. Kapp, and J. Eisner, British Journal of Dermatology, vol. 153, no. 4, pp. 849-851, 2005; and Lacouture M E. Nat Rev Cancer. 2006; 6:803-812.

Oral tetracyclines have been shown to be partially useful in the management of EGFR inhibitor associated rash. Randomized trials have exhibited the beneficial use of oral doxycycline and minocycline in the treatment of skin rash in patients receiving EGFR-targeted therapies. For example, a controlled study called STEPP (Skin Toxicity Evaluation Protocol with Panitumumab) was the first prospective trial designed specifically to compare primary pre-emptive treatment with reactive treatment for EGFRI-mediated skin toxicity. In the absence of any proven drugs, or FDA-approved drugs for this adverse toxicity, treatment is limited to an approved modality, including: moisturizer, sunscreen, 1% hydrocortisone cream, and oral antibiotics. Results indicated that, as compared with reactive treatment (received after development of skin toxicity), pre-emptive treatment (received 24 hours before the first dose of panitumumab through week 6) reduced the incidence of grade 2 or greater skin toxicities by more than 50% without additional side effects. In addition, time to severe skin toxicity was significantly delayed in the pre-emptive treatment arm. The time to first occurrence of any grade 2 or greater skin toxicity was also significantly delayed in the pre-emptive arm. See, e.g., Mario E. Lacouture, J Clin Oncol. 2010 Mar. 10; 28(8):1351-7.

Two randomized double-blind trials have examined the effects of prophylactic skin rash treatment. See, e.g., Scope A, Agero A L, Dusza S W, et al. J Clin Oncol 2007; 25:5390-6; Jatoi A, Rowland K, Sloan J A, et al. Cancer 2008; 113:847-53. An 8-week trial studied prophylactic oral minocycline as compared with placebo for patients with metastatic CRC preparing to initiate cetuximab therapy. At weeks 1-4 of mAb treatment, the minocycline group had a significantly lower total facial lesion count and a significantly reduced incidence of moderate-to-severe itch as compared with the placebo group. In another double-blind trial, patients starting EGFRI therapy were randomized to tetracycline (500 mg twice daily) or to placebo for 4 weeks. Although tetracycline did not prevent EGFRI-induced rash, a reduction in rash severity was observed. At week 4, grade 2 rash was reported in 17% of the tetracycline group and in 55% of the placebo group. Treatment also improved certain SKINDEX-16 quality-of-life measures, including skin burning or stinging and skin irritation.

While both oral doxycycline and oral minocycline help mitigate rash in EGFRI-treated patients, their benefit is hindered by (i) the inherent systemic side effects of antibiotics; and (ii) certain cases of drug-drug interactions.

Several topical treatments have also been tested but their success was limited. A Placebo-Controlled Trial from the North Central Cancer Treatment Group (N05C4) was undertaken to determine whether sunscreen prevents or mitigates EGFR inhibitor-induced rashes. In this trial, fifty-four patients received sunscreen, and 56 received placebo. There were no significant differences in rash severity, and patient-reported outcomes of rash yielded similar conclusions. Sunscreen, as prescribed in this trial, did not prevent or attenuate EGFR inhibitor-induced rash. See, e.g., Amina Jatoi, The Oncologist 2010; 15:1016-1022.

In a randomized double blind trial of prophylactic oral minocycline treatment for EGFR inhibitor-induced rash, patients were also instructed to apply tazarotene cream twice a day. There was no observed clinical benefit to the tazarotene application. Tazarotene treatment was associated with significant irritation, causing its discontinuation in one-third of the patients. The rash was even assessed as more severe in 10% of the patients applying tazarotene. See, e.g., Scope, A. L. C. Agero, S. W. Dusza et al., Journal of Clinical Oncology, vol. 25, no. 34, pp. 5390-5396, 2007.

Scope et al. conducted a half face study to evaluate whether pimecrolimus could reduce acne-like eruption as well as rash severity induced by cetuximab. After 2 weeks, lesion counts were significantly less in the pimecrolimus treated side. This benefit was maintained to week 5. However, there was a trend towards reduced lesion count on both sides of the face. Moreover, no significant difference in rash severity and patient assessment of symptoms was observed. See, e.g., A. Scope, J. A. Lieb, S. W. Dusza et al., Journal of the American Academy of Dermatology, vol. 61, no. 4, pp. 614-620, 2009.

Wong et al. evaluated the effect of Regenecare gel, which includes 2% lidocaine, aloe vera, marine collagen, and sodium alginate, on skin toxicity induced by various types of EGFRIs. Regenecare gel was applied to the right side of the face for 1 week and later applied to the entire face. There was a significant improvement in itchiness. However, the authors did not provide any information about its impact on skin toxicity. See, e.g., S. Wong, K. Osann, A. Lindgren, T. Byun, and M. Mummaneni, Journal of Clinical Oncology, vol. 26, Article ID 20507, 2008.

Moreover, tetracycline, antibiotics, such as tetracycline, oxytetracycline, demeclocycline, doxycycline, lymecycline, meclocycline, methacycline, minocycline, rolitetracycline, chlorotetracycline and tigecycline, are extremely unstable compounds and are sensitive to many formulation excipients (for example, water, short chain alcohols, certain polymers, certain hydrophilic solvents, and surfactants). Thus, most tetracyclines, e.g., minocycline and doxycycline, currently exist only in solid oral dosage forms or are given by injection or infusion.

A product that requires a short treatment period, which is safe, well tolerated, and prevents occurrence and/or reduces the grade of severity or the incidences of EGFRI-induced rash would be advantageous and could improve patient compliance with EGFRI treatment.

### SUMMARY

In one or more embodiments, there is provided a topical composition comprising a tetracycline antibiotic to counteract or ameliorate dermal side effects, or adverse effects, of EGFR inhibitors. The term side effect is used interchangeably with the term adverse effect.

In one or more embodiments, the effect of administering a composition comprising a tetracycline antibiotic is achieved by delivering the tetracycline antibiotic onto and into the skin or mucosa or follicles. In one or more embodiments, systemic penetration through the skin, mucosa or follicles is low. In one or more embodiments, systemic penetration through the skin, mucosa or follicles is less than about 10%, less than about 9%, less than about 8%, less than about 7%, less than about 6%, less than about 5%, less than about 4%, less than about 3%, less than about 2%, less than about 1% or less than about 0.5% of the tetracycline antibiotic applied to the skin. In one or more embodiments, the average maximum systemic penetration through the skin, mucosa or follicles is less than 5 ng/mL or about 5 ng/mL. In one or more embodiments, the maximum systemic penetration through the skin mucosa or follicles is between about 1.5 ng/mL to about 6.2 ng/mL. In one or more embodiments, systemic delivery or systemic penetration through the skin, mucosa or follicles can supplement the effects produced by non-systemic delivery onto and into the skin, mucosa or follicles.

In one or more embodiments, the maximum plasma concentration for doxycycline is less than 5 ng/mL following administration of a doxycycline composition provided herein.

In one or more embodiments, the maximum plasma concentration for doxycycline is about 5 ng/mL following administration of a doxycycline composition provided herein.

In one or more embodiments, the maximum plasma concentration for doxycycline is less than the concentration obtained for a similarly formulated minocycline composition following administration of the compositions.

In one or more embodiments, the maximum plasma concentration for doxycycline is higher than the concentration obtained for a similarly formulated minocycline composition following administration of the compositions.

In one or more embodiments, the maximum plasma concentration for doxycycline will be about the same as the concentration obtained for a similarly formulated minocycline composition following administration of the compositions.

In one or more embodiments, the tetracycline antibiotic may, without being bound by any theory, act in a way directly or indirectly to affect EGFR receptors in the skin, mucosa or follicles so as, for example, to help partially or fully return or restore skin, mucosa or follicle function or cycle to normal. Successful topical treatment or amelioration (prophylactically or otherwise) of a systemically induced rash is surprising when the source of the rash is systemic. In one or more embodiments, a composition comprising a tetracycline antibiotic is administered topically.

For example, topical hydrophobic therapeutic breakable gel and foamable compositions comprising tetracycline, including those without surfactants, have been described, for example in U.S. application Ser. Nos. 13/499,501, 13/499,727, 13/499,475, and 13/499,709, U.S. Publication No. 2011/0281827, WO 11/039637, WO 11/039638, WO 11/138678 and WO 2011/064631, all of which are herein incorporated in their entirety by reference. More particularly, any of the active ingredients, carriers, solvents, surfactants, foam adjuvants, fatty acids, fatty alcohols, polymeric agents, penetration enhancers, preservatives, humectants, moisturizers, and other excipients, as well as the propellants and methods listed therein can be applied herein and are incorporated by reference.

Methods for treatment of impetigo and acne and accelerating skin restoration and wound healing using topical therapeutic gel and foamable compositions comprising tetracycline have been described, for example in U.S. application Ser. Nos. 13/831,396, 14/147,376, 14/147,401, 14/384,978 and PCT/US2013/031387, all of which are herein incorporated in their entirety by reference. More particularly, any of the active ingredients, carriers, solvents, surfactants, foam adjuvants, fatty acids, fatty alcohols, polymeric agents, penetration enhancers, preservatives, humectants, moisturizers, and other excipients, as well as the propellants and methods listed therein can be applied herein and are incorporated by reference.

In one or more embodiments, the tetracycline antibiotic is micronized. In one or more embodiments, it is encapsulated. In one or more embodiments, the active agent is encapsulated in particles, microparticles, nanoparticles, microcapsules, microspheres, nanocapsules, nanospheres, liposomes, niosomes, polymer matrices, silica-gels, graphite, nanocrystals, or microsponges. Such particles can have various functions, such as (1) protection of the drug from degradation; (2) modification of the drug release rate from the composition; (3) control of skin penetration profile; and (4) mitigation of adverse effects, due to the controlled release of the active agent from the encapsulation particles. Encapsulation is described in U.S. Publication No. 2015/0209296, which is incorporated by reference. In one or more embodiments related to one or more of the foregoing, the tetracycline active ingredient is associated with solid, porous microcarriers, each having a hydrophobic surface. In one or more additional embodiments, the solid, porous microcarriers comprise a material selected from the group consisting of hydrophobic surface-modified silicon dioxide, porous polystyrene, porous polyamide, porous hydrophobic cellulose, and porous polytetrafluoroethylene. In one or more embodiments, the microcarrier possesses a porous structure for retaining the active ingredient, a hydrophobic surface, and is chemically non-reactive with the active ingredient. In one or more additional embodiments, the hydrophobic encapsulant comprises a material selected from the group consisting of mineral oil, petrolatum jelly, synthetic waxes, natural waxes, and silicone oils. In one or more embodiments, the average encapsulant particle size is below 95 microns, is below 75 microns, is below 50 microns, or is below 25 microns. In one or more embodiments, the average particle size of the tetracycline antibiotic is below 22 microns, is below 15 microns, is about 5.5 to about 10.5 microns, is about 6 microns to about 10.5 microns, is about 6.5 to about 10 microns, is about 7 to about 9.5 microns, or is about 7.5 to about 9 microns.

In one or more embodiments, the composition is a gel, paste, lotion, cream, soap, spray, mask, patch, powder, pomade, ointment, oil, foam or mousse. In one or more embodiments, the composition is hydrophobic. In one or more embodiments, the composition comprises hydrophobic oils and waxes. In one or more embodiments, the composition comprises fatty alcohols. In one or more embodiments, the composition comprises hydrophobic oils and waxes. In one or more embodiments, the composition comprises fatty acids. In one or more embodiments, the composition is surfactant free. In one or more embodiments, the composition is given as an adjunct to treatment with an EGFR inhibitor. In one or more embodiments, the EGFR inhibitor is an antibody. In one or more embodiments, the antibody is a monoclonal antibody such as cetuximab, panitumumab, zalutumumab, nimotuzumab, or matuzumab. In one or more embodiments, the inhibitor targets EGFR tyrosine kinase, such as erlotinib, gefitinib, lapatinib, canertinib or vandetanib.

In one or more embodiments, the composition is given prophylactically before onset of EGFR inhibitor therapy. In one or more embodiments, the composition is administered at the beginning of inhibitor therapy. In one or more embodiments, the composition is administered in parallel with inhibitor therapy. In one or more embodiments, the composition is administered after the beginning of inhibitor therapy. In one or more embodiments, the composition is administered during the first week, first two weeks, first three weeks, first month, first five weeks, first six weeks, first seven weeks, first eight weeks, first nine weeks, first ten weeks, first eleven weeks or first twelve weeks of inhibitor therapy or some similar period, which could include part of a week, such as one day, two days, three days, four days, five days, or six days. In one or more embodiments, the composition is administered one, two, three, four, five, six, seven, or eight weeks prior to the beginning of inhibitor therapy.

Applicants conducted a randomized, double blind, vehicle controlled study to ascertain whether topical doxycycline foam can be used instead of prophylactic oral medication that potentially entails systemic side effects, and to ascertain safety as well as preliminary efficacy in this prospective (see Example 6). The foam was administered topically twice daily for prevention of EGFRI skin toxicity, to patients with advanced cancer receiving cetuximab or panitumumab. A clear treatment benefit was observed in patients administered a topical doxycycline formulation (FDX104, Example 2, Table 3D) as compared to administration of placebo.

In one or more embodiments, there is provided a method for preventing or treating an EGFR inhibitor induced skin, nail, or mucosal disorder in a subject, comprising topically administering prior to and/or during systemic administration of the EGFR inhibitor a topical composition comprising a tetracycline antibiotic to at least a portion of the skin, nail, or mucosa of the subject. In some embodiments, the composition comprises a carrier and a tetracycline antibiotic. In some embodiments, the composition comprises a carrier and a tetracycline antibiotic and an additional active agent. In some embodiments, the composition comprises a propellant and a foamable composition comprising a carrier and a tetracycline antibiotic. In some embodiments, the composition comprises a propellant and a foamable composition comprising a carrier and a tetracycline antibiotic and an additional active agent.

In one or more embodiments, there is provided a method for preventing or treating an EGFR inhibitor induced skin, nail, or mucosal disorder in a subject, comprising topically administering prior to and/or during systemic administration of the EGFR inhibitor a topical composition comprising a hydrophobic solvent and a tetracycline antibiotic to at least a portion of the skin, nail, or mucosa of the subject.

In one or more embodiments, there is provided a method for preventing or treating an EGFR inhibitor induced skin, nail, or mucosal disorder in a subject, comprising topically administering prior to and/or during systemic administration of the EGFR inhibitor a topical composition comprising a hydrophobic solvent, a fatty alcohol, and a tetracycline antibiotic to at least a portion of the skin, nail, or mucosa of the subject.

In one or more embodiments, there is provided a method for preventing or treating an EGFR inhibitor induced skin, nail, or mucosal disorder in a subject, comprising topically administering prior to and/or during systemic administration of the EGFR inhibitor a topical composition comprising a hydrophobic solvent, a fatty acid, and a tetracycline antibiotic to at least a portion of the skin, nail, or mucosa of the subject.

In one or more embodiments, there is provided a method for preventing or treating an EGFR inhibitor induced skin, nail, or mucosal disorder in a subject, comprising topically administering prior to and/or during systemic administration of the EGFR inhibitor a topical composition comprising a hydrophobic solvent, a fatty acid, a fatty alcohol, and a tetracycline antibiotic to at least a portion of the skin, nail, or mucosa of the subject.

In one or more embodiments, there is provided a method for preventing or treating an EGFR inhibitor induced skin, nail, or mucosal disorder in a subject, comprising topically administering prior to and/or during systemic administration of the EGFR inhibitor a topical composition comprising a hydrophobic solvent, a wax, and a tetracycline antibiotic to at least a portion of the skin, nail, or mucosa of the subject.

In one or more embodiments, there is provided a method for preventing or treating an EGFR inhibitor induced skin, nail, or mucosal disorder in a subject, comprising topically administering prior to and/or during systemic administration of the EGFR inhibitor a topical composition comprising a fatty acid and/or a fatty alcohol, a wax, a tetracycline antibiotic, and a hydrophobic solvent, to at least a portion of the skin, nail, or mucosa of the subject.

In one or more embodiments, there is provided a method for preventing or treating an EGFR inhibitor induced skin, nail, or mucosal disorder in a subject, comprising topically administering prior to and/or during systemic administration of the EGFR inhibitor a topical composition comprising a wax, and a tetracycline antibiotic to at least a portion of the skin, nail, or mucosa of the subject.

In one or more embodiments, there is provided a method for preventing or treating an EGFR inhibitor induced skin, nail, or mucosal disorder in a subject, comprising topically administering prior to and/or during systemic administration of the EGFR inhibitor a topical composition comprising a fatty acid and/or a fatty alcohol and a tetracycline antibiotic to at least a portion of the skin, nail, or mucosa of the subject.

In one or more embodiments, there is provided a method for preventing or treating an EGFR inhibitor induced skin, nail, or mucosal disorder in a subject, comprising topically administering prior to and/or during systemic administration of the EGFR inhibitor a topical composition comprising a fatty acid and/or a fatty alcohol, a wax, and a tetracycline antibiotic to at least a portion of the skin, nail, or mucosa of the subject.

In one or more embodiments, there is provided a method for preventing or treating an EGFR inhibitor induced skin, nail, or mucosal disorder in a subject, comprising topically administering prior to and/or during systemic administration of the EGFR inhibitor a topical composition comprising a wax, a tetracycline antibiotic, an additional active agent to at least a portion of the skin, nail, or mucosa of the subject.

In one or more embodiments, there is provided a method for preventing or treating an EGFR inhibitor induced skin, nail, or mucosal disorder in a subject, comprising topically administering prior to and/or during systemic administration of the EGFR inhibitor a topical composition comprising a fatty acid and/or a fatty alcohol, a tetracycline antibiotic, and an additional active agent to at least a portion of the skin, nail, or mucosa of the subject.

In one or more embodiments, there is provided a method for preventing or treating an EGFR inhibitor induced skin, nail, or mucosal disorder in a subject, comprising topically administering prior to and/or during systemic administration of the EGFR inhibitor a topical composition comprising a fatty acid and/or a fatty alcohol, a wax, a tetracycline antibiotic, and an additional active agent to at least a portion of the skin, nail, or mucosa of the subject.

In one or more embodiments, there is provided a method for preventing or treating an EGFR inhibitor induced skin, nail, or mucosal disorder in a subject, comprising topically administering prior to and/or during systemic administration of the EGFR inhibitor a topical composition comprising a fatty acid and/or a fatty alcohol, a wax, a tetracycline antibiotic, an additional active agent, and a hydrophobic solvent, to at least a portion of the skin, nail, or mucosa of the subject. In one or more embodiments the additional active agent is selected from the group consisting of an antihistamine, a corticosteroid, a retinoid, and a tricyclic antidepressant. In some embodiments the additional active agent is doxepin or adapalene.

In one or more embodiments, the antihistamine is, for example, astemizole, azatadine, azelastine, bromodiphenhydramine, brompheniramine, carbinoxamine, cetirizine, chlorocyclizine, clemastine, chlorothen, cyclizine, cyproheptadine, desloratadine, dexbrompheniramine, dimethindene, diphenylpyraline, doxylamine, fexofenadine, hydroxyzine, isothipendyl, loratadine, methapyrilene, montelukast, phenindamine, pheniramine, phenyltoloxamine, prophenpyridamine, pyrilamine, terfenadine, thenyldiamine, thonzylamine, trimeprazine, triprolidine and pharmaceutically acceptable salts thereof such as, e.g., azatadine maleate, fexofenadine HCl, hydroxyine HCl, isothipendyl HCl (theruhistin), methapyrilene HCl, montelukast sodium, tartrate, pheniramine maleate, phenyltoloxamine citrate, prophenpyridamine maleate, pyrilamine maleate, thenyldiamine HCl, trimeprazine, triprolidine HCl, buclizine, desloratidine, ebastine, emedastine, epinastine, ketotifen, levocabastine, levocetirizine, loratidine, mequitazine, mizolastine, olopatadine, oxatomide, terfenidine, pharmaceutically acceptable salts, isomers or prodrugs thereof, mepyramine, antazoline, dimenhydrinate, meclizine, thenaldine, alimemazine, ketotifen, acrivastine, embramine, dexchlorpheniramine, diphenhydramine, misolastine, phenidamine, diphenhydramine, doxepin, phrilamine maleate, chlorpheniramine, tripelennamine, phenothiazine, promethazine hydrochloride, dimethindene maleate or mixtures of any two or more thereof.

In one or more embodiments, the corticosteroid is, for example, acetonide, aclometasone dipropionate, aldosterone, alpha-methyl dexamethasone, amcinafel, amcinafide, amcinonide, beclomethasone, beclomethasone dipropionates, betamethasone, betamethasone diproprionate, betamethasone sodium phosphate, betamethasone valerate, bronchodialator, budesonide, chloroprednisone, chloroprednisone acetate, ciclesonide, clescinolone, clobetasol proprionate, clobetasol valerate, clobetasol valerate, clobetasol-17-propionate, clobetasone-17-butyrate, clocortelone, cortiso, cortisone, cortisone acetate, cortisone, dexamethasone, cortodoxone, deflazacort, defluprednate, desoxycorticosterone acetate, desoxymethasone, dexamethasone, dexamethasone sodium phosphate, dexamethasone-phosphate, dichlorisone, diflorasone diacetate, diflucortolone valerate, diflurprednate, dipropionate HFA, fluadrenolone, flucetonide, fluclorolone acetonide, flucloronide, flucortine butylesters, flucortine butylesters, flucortolone, flucortolone caproate, fludrocortisone, flumethasone pivalate, flunisolide, fluocinolone acetonide, fluocinonide, fluocortolone, fluocortolone hydrocortisone-17-valerate, fluocortolone caproate, fluocortolone pivalate, fluoromethalone, fluosinolone acetonide, fluosinolone acetonide, fluperolone, fluprednidene (fluprednylidene) acetate, fluprednidene acetate, fluprednisolone, fluradrenolone, fluradrenolone acetonide, fluticasone, fluticasone furoate, fluticasone propionate, formoterol, halcinonide, hydrocortisone valerate, halobetasol proprionate, halometasone, hydrocortamate, hydrocortisone, hydrocortisone acetate, hydrocortisone butyrate, hydrocortisone cyclopentylpropionate, hydrocortisone valerate, hydrocortisone; budesonide, hydrocortisone-17-aceponate, hydrocortisone-17-buteprate, Hydrocortisone-17-butyrate, hydroxyl-triamcinolone, medrysone, meprednisone, methylprednisolone, mometasone, Mometasone furoate, paramethasone, prednicarbate, clobetasone-17-butyrate, prednisolone, prednisone, prednisone hydrocortisone acetat, rofleponide, Salmeterol, tixocortol, tixocortol pivalate, tixocortol prednisolone, triamcinolone, triamcinolone acetonide, triamcinolone alcohol, triamcinolone hexacatonide or mixtures of any two or more thereof.

In one or more embodiments, the retinoid is, for example, retinol, retinal, all trans retinoic acid and derivatives, isomers and analogs thereof, etretinate, actiretin, isotretinoin, adapalene, tazarotene, tretinoin, alitretinoin, seletinoid G or mixtures of any two or more thereof.

Suitable, but non-limiting, retinoids for use in the present invention are listed below.

It is convenient to omit the explicit representation of C and H atoms in the parent skeletal structure of retinoids as follows:

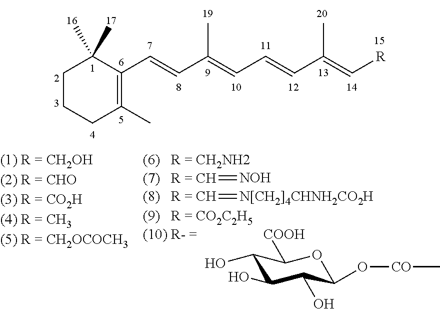

Compound (1) (2E,4E,6E,8E)-3,7-dimethyl-9-(2,6,6-trimethylcyclohex-1-en-1-yl)nona-2,4,6,8-tetraen-1-ol is also known as vitamin A, vitamin A alcohol, retinal, vitamin A₁, vitamin A₁ alcohol, axerophthol or axerol. Compound (2) also known as vitamin A aldehyde, vitamin A₁ aldehyde, retinene or retinene₁ and retinal or, if liable to be confused with the adjective retinal (pertaining to the retina), retinaldehyde. Compound (3) also known as tretinoin (see note), vitamin A acid or vitamin A₁ acid should be designated retinoic acid. Compound (4), is known as axerophthene. Functional substitution at the 15 position of the basic hydrocarbon is denoted by the use of the group names retinyl (R is CH₂—) or retinylidene (R is CH═), with retention of the original numbering of the basic hydrocarbon. For example, compound (5) is retinyl acetate and (6) is retinylamine. Derivatives of retinal include for example Compound (7)—retinal oxime and Compound (8)—N⁶-retinylidene-L-lysine. Other derivatives of retinoic acid, named as carboxylic acid derivatives Compound (9)—ethyl retinoate and Compound (10)—1-O-retinoyl-b-D-glucopyranuronic acid.

Retinoids that differ in hydrogenation level from the parent structure (displayed above) are named by use of the prefixes ‘hydro’ and ‘dehydro’ together with locants specifying the carbon atoms at which hydrogen atoms have been added or removed. Examples of such retinoid compounds are Compound (11)—3,4-Didehydroretinol (also known as dehydroretinol or vitamin A₂) and Compound (12)—4,5-Didehydro-5,6-dihydroretinol (also known as alpha-vitamin A).

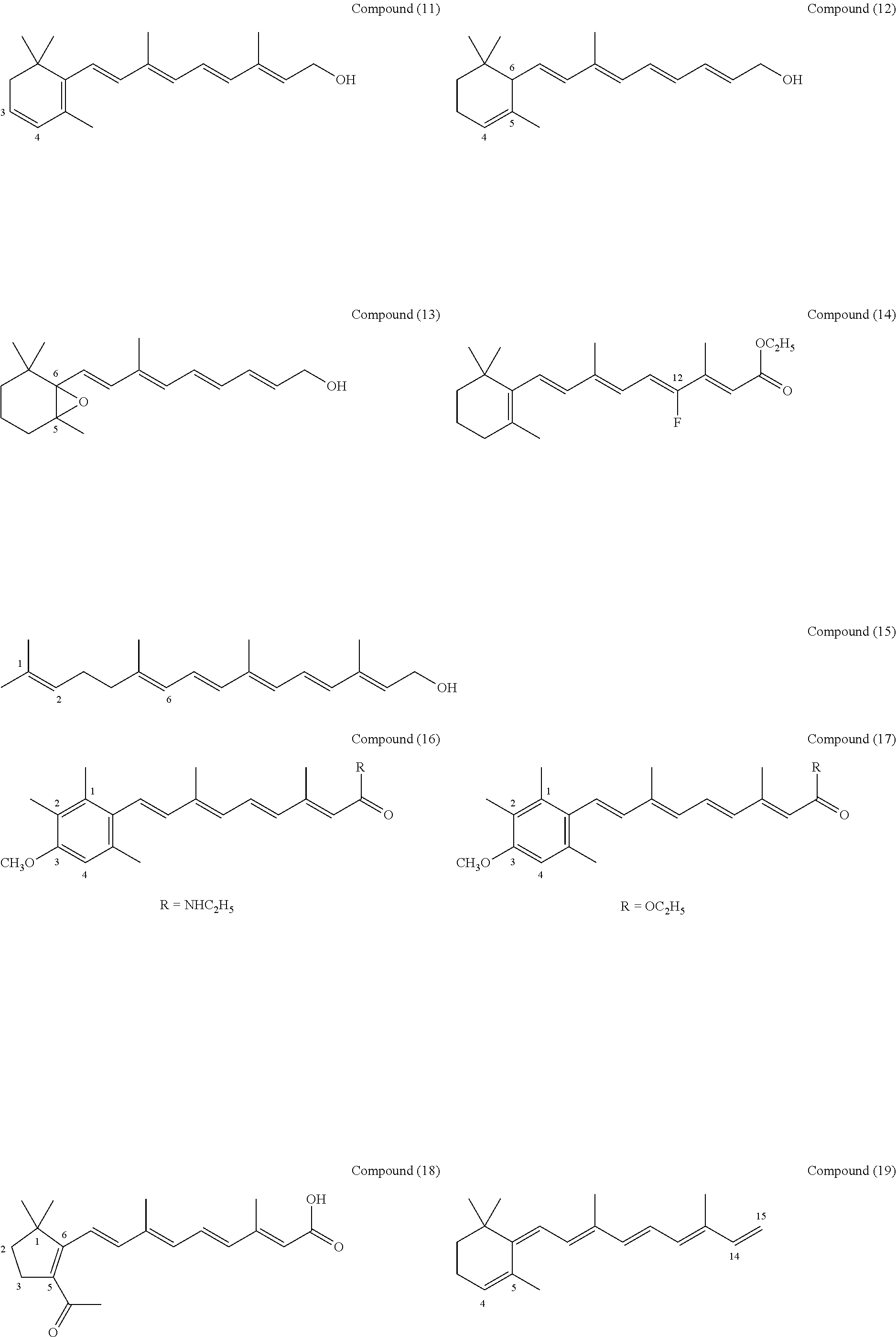

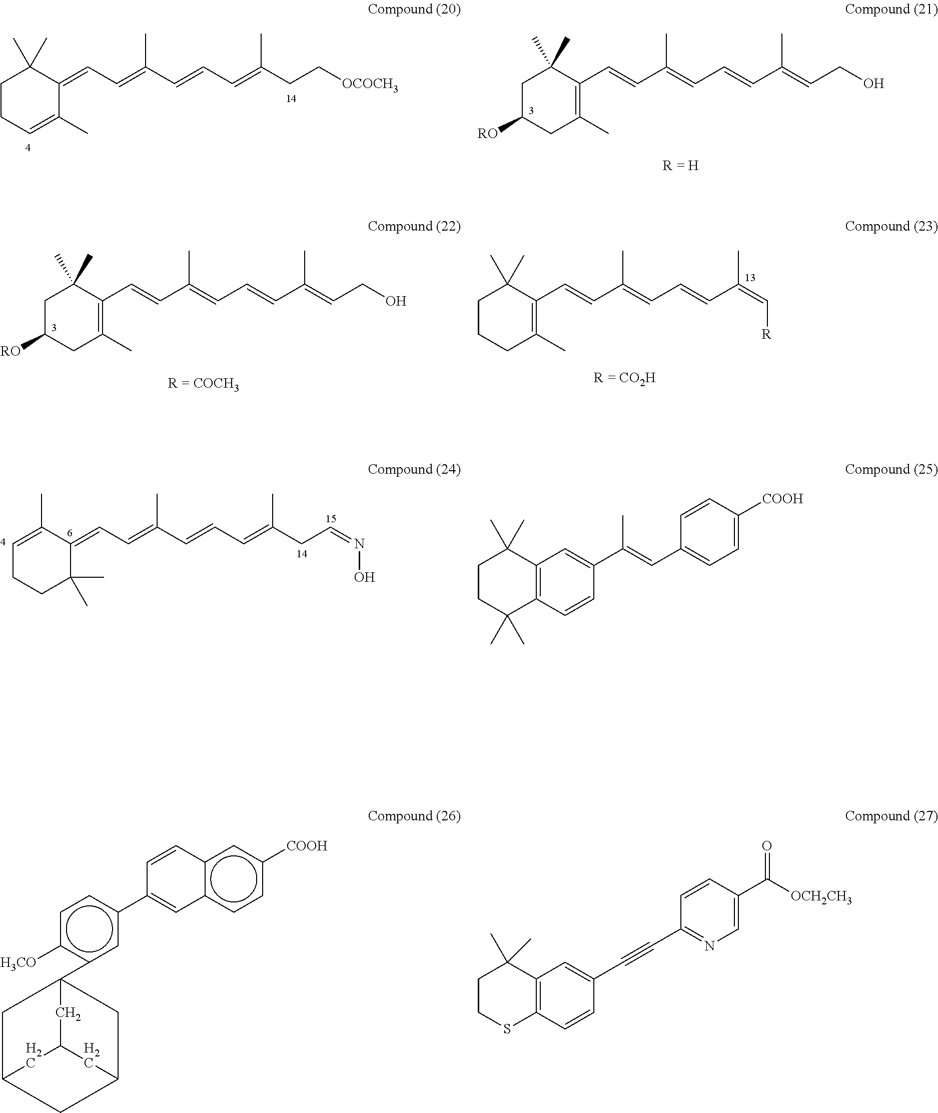

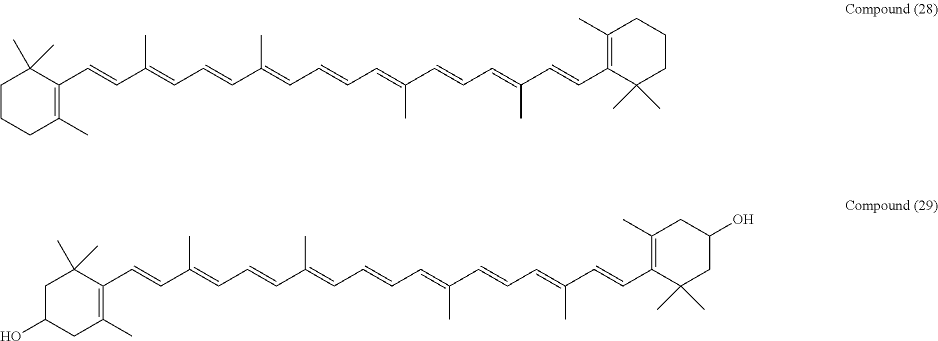

Substituted derivatives of retinoids are exemplified by Compound (13)—5,6-Epoxy-5,6-dihydroretinol (also known as hepaxanthin) and Compound (14)—Ethyl 12-fluororetinoate. Seco Retinoids are exemplified by Compound (15)—1,6-Seco-1,2-didehydroretinol, also known as g-vitamin A, and Nor Retinoids, which result from the elimination of a CH₃, CH₂, CH or C group from a retinoid are exemplified by Compound (16)—N-Ethyl-3-methoxy-2-methyl-17-nor-1,2,3,4-tetradehydroretinamide (also known as motretinide), Compound (17)—Ethyl 3-methoxy-2-methyl-17-nor-1,2,3,4-tetradehydroretinoate (also known as etretinate), acitretin (Compound (17), wherein R═H) and Compound (18)—5-Acetyl-4,18-dinor-retinoic acid. Retro Retinoids are exemplified by Compound (19)—4,5-Didehydro-5,5-retro-deoxyretinol (also known as anhydro vitamin A and Compound (20)—4,14-retro-Retinyl acetate. Stereoisomers of retinoids are exemplified by Compound (21)—(3R)-3-Hydroxyretinol and Compound (22)—(3R)-3-Acetoxyretinol. Other stereochemical isomers can are exemplified by Compound (23)—13-cis-Retinoic acid or (7E,9E,11E,13Z)-retinoic acid (also known as isotretinoin) and Compound (24)—(6E,8E,10E,12E,15Z)-4,14-retro-Retinaloxime.

“Arotinoids” or “retinoidal benzoic acid derivatives” contain, aromatic rings replacing either the basic β-ionone type ring structure or unsaturated bonds of the tetraene side chain of the parent retinoid skeleton, as exemplified by Compound (25) and Compound (26)—6-[3-(1-adamantyl)-4-methoxyphenyl]-2-naphthoic acid, also known as adapalene. Several artinoids, possessing potent retinoid properties, including but not limited to short retinoids, short heterocyclic retinoids, isoxazole-containing retinoids, heterocyclic isoxazole-containing retinoids, isoxazoline-containing retinoids, stilbene retinoid analogs, are disclosed in Pure Appl. Chem., Vol. 73, No. 9, pp. 1437-1444, 2001.

Tazarotene (Ethyl 6-[2-(4,4-dimethylthiochroman-6-yl)ethynyl] nicotinate) is exemplary to a retinoid precursor—Compound (27), suitable as retinoid for use in the present invention.

Yet, other non-limiting exemplary retinoid precursors are carotenes, such as all-trans beta carotene—Compound (28), alpha carotene, lycopene and 9-cis-beta-carotene, as well as xanthophils (also termed “oxicarotenoids”), such as lutein and zeaxanthin—Compound (29).

Salts and derivatives of retinoid compounds are also suitable as “retinoid” for use in the present invention.

Retinoid compounds can be ascertained recognized and identified by methods known in the art. One method involves the use of competitive nuclear retinoic acid (RA and RX) receptor binding assays for identifying compounds which bind directly to the receptors. For instance, J. J. Repa et al., “All-trans-retinol is a ligand for the retinoic acid receptors”, Proc. Natl. Acad. Sci. USA, Vol. 90, pp. 7293-7297, 1993, discloses a competitive RA receptor binding assay based on human neuroblastoma cell nuclear extracts. H. Torma et al. ((1994) “Biologic activities of retinoic acid and 3,4-dehydroretinoic acid in human keratinoacytes are similar and correlate with receptor affinities and transactivation properties,” J. Invest. Dermatology, Vol. 102, pp. 49-54) discloses assays for measuring binding affinities for the nuclear retinoic acid receptors and for measuring transcriptional activation induction. M. F. Boehm et al. ((1994) “Synthesis of high specific activity [.sup.3 H]-9-cis-retinoic acid and its application for identifying retinoids with unusual binding properties,” J. Med. Chem., Vol. 37, pp. 408-414) discloses a ligand-binding assay and a receptor/reporter cotransfection assay for monitor regulation of gene expression. EP 0 552 612 A2, published Jul. 28, 1993, describes ligand-binding trapping assays based on incubation of radiolabeled compounds with transfected COS-1 cells which express RA and RX receptors.

Mixtures of these retinoids can also be employed according to the present invention.

Suitable retinoids include, but are not limited to, retinol, retinal, retinoic acid, all-trans retinoic acid, isotretinoin, tazarotene, adapalene, 13-cis-retinoic acid, acitretin all-trans beta carotene, alpha carotene, lycopene, 9-cis-beta-carotene, lutein and zeaxanthin.

In one or more embodiments, the tricyclic antidepressant is, for example, amitriptyline, desipramine, doxepine, imipramine, nortriptyline, amoxapine, clomipramine, maprotiline, trimipramine, protriptyline, or mixtures of any two or more thereof.

In one or more embodiments an active agent for use in the compositions provided or described herein is for example, imipramine, alprazolam, amitriptyline, amoxapine, benzodiazepine, bupivacaine, butriptyline, carbamazepine, chlordiazepoxide, clomipramine, clonazepam, desipramine, diazepam, dothiepin, doxepin, duloxetine, flecainide, flurazepam, fluvoxamine, halazepam, imipramine, iprindole, isocarboxazid, levobupivacaine, levodopa, lidocaine, lithium, lofepramine, maprotiline, nortriptyline, opioid, paroxetine, phenelzine, prazepam, proparacaine, protriptylin, protriptyline, ropivacaine, serotonin, tetracaine, tranylcyclopramine, trimipramine, valproate or mixtures of any two or more thereof.

Other carriers and compositions are described in: U.S. Publication No. 2005/0232869, published on Oct. 20, 2005, entitled NONSTEROIDAL IMMUNOMODULATING KIT AND COMPOSITION AND USES THEREOF; U.S. Publication No. 2005/0205086, published on Sep. 22, 2005, entitled RETINOID IMMUNOMODULATING KIT AND COMPOSITION AND USES THEREOF; U.S. Publication No. 2006/0018937, published on Jan. 26, 2006, entitled STEROID KIT AND FOAMABLE COMPOSITION AND USES THEREOF; U.S. Publication No. 2005/0271596, published on Dec. 8, 2005, entitled VASOACTIVE KIT AND COMPOSITION AND USES THEREOF; U.S. Publication No. 2006/0269485, published on Nov. 30, 2006, entitled ANTIBIOTIC KIT AND COMPOSITION AND USES THEREOF; U.S. Publication No. 2007/0292355, published on Dec. 20, 2007, entitled ANTI-INFECTION AUGMENTATION OF FOAMABLE COMPOSITIONS AND KIT AND USES THEREOF; U.S. Publication No. 2008/0317679, published on Dec. 25, 2008, entitled FOAMABLE COMPOSITIONS AND KITS COMPRISING ONE OR MORE OF A CHANNEL AGENT, A CHOLINERGIC AGENT, A NITRIC OXIDE DONOR, AND RELATED AGENTS AND THEIR USES; U.S. Publication No. 2008/0044444, published on Feb. 21, 2008, entitled DICARBOXYLIC ACID FOAMABLE VEHICLE AND PHARMACEUTICAL COMPOSITIONS THEREOF; U.S. Publication No. 2008/0069779, published on Mar. 20, 2008, entitled FOAMABLE VEHICLE AND VITAMIN AND FLAVONOID PHARMACEUTICAL, COMPOSITIONS THEREOF; U.S. Publication No. 2008/0206159, published on Aug. 28, 2008, entitled COMPOSITIONS WITH MODULATING AGENTS; U.S. Publication No. 2008/0206161, published on Aug. 28, 2008, entitled QUIESCENT FOAMABLE COMPOSITIONS, STEROIDS, KITS AND USES THEREOF; U.S. Publication No. 2008/0260655, published on Oct. 23, 2008, entitled SUBSTANTIALLY NON-AQUEOUS FOAMABLE PETROLATUM BASED PHARMACEUTICAL AND COSMETIC COMPOSITIONS AND THEIR USES; U.S. Publication No. 2011/0268665, published on Nov. 3, 2011, entitled OIL-BASED FOAMABLE CARRIERS AND FORMULATIONS; U.S. Publication No. 2012/0087872, published on Apr. 12, 2012, entitled FOAMABLE VEHICLES AND PHARMACEUTICAL COMPOSITIONS COMPRISING APROTIC POLAR SOLVENTS AND USES THEREOF; U.S. Publication No. 2012/0213709, published on Aug. 23, 2012, entitled NON SURFACTANT HYDRO-ALCOHOLIC FOAMABLE COMPOSITIONS, BREAKABLE FOAMS AND THEIR USES; U.S. Publication No. 2012/0213710, published on Aug. 23, 2012, entitled SURFACE ACTIVE AGENT NON POLYMERIC AGENT HYDRO-ALCOHOLIC FOAMABLE COMPOSITIONS, BREAKABLE FOAMS AND THEIR USES; U.S. Publication No. 2013/0064777, published on Mar. 14, 2013, entitled SURFACTANT-FREE WATER-FREE FOAMABLE COMPOSITIONS, BREAKABLE FOAMS AND GELS AND THEIR USES; U.S. Publication No. 2013/0053353, published on Feb. 28, 2013, entitled COMPOSITIONS, GELS AND FOAMS WITH RHEOLOGY MODULATORS AND USES THEREOF; U.S. Publication No. 2011/0281827, published on Nov. 17, 2011, entitled COMPOSITIONS, GELS AND FOAMS WITH RHEOLOGY MODULATORS AND USES THEREOF; U.S. Publication No. 2013/0028850, published on Jan. 31, 2013, entitled TOPICAL TETRACYCLINE COMPOSITIONS; U.S. Publication No. 2013/0011342, published on Jan. 10, 2013, entitled SURFACTANT-FREE, WATER-FREE, FOAMABLE COMPOSITIONS AND BREAKABLE FOAMS AND THEIR USES; U.S. Publication No. 2013/0225536, published on Aug. 29, 2013, entitled COMPOSITIONS FOR THE IMPROVED TREATMENT OF ACNE AND RELATED DISORDERS; U.S. Publication No. 2014/0121188, published on May 1, 2014, entitled METHODS FOR ACCELERATED RETURN OF SKIN INTEGRITY AND FOR THE TREATMENT OF IMPETIGO; U.S. Publication No. 2015/0164922, published on Jun. 18, 2015, entitled USE OF TETRACYCLINE COMPOSITIONS FOR WOUND TREATMENT AND SKIN RESTORATION, all of which are incorporated herein by reference in their entirety. More particularly, any of the active ingredients, carriers, solvents, surfactants, foam adjuvants, polymeric agents, penetration enhancers, preservatives, humectants, moisturizers, and other excipients, as well as the propellants and methods listed therein can be applied herein and are incorporated by reference.

In one or more embodiments, there is provided a method for reducing the risk of skin, nail, or mucosal side effects associated with systemic EGFR inhibitor treatment in a subject, the method comprising topically administering prior to and/or during the systemic EGFR inhibitor administration a topical composition comprising a tetracycline antibiotic to at least a portion of the skin, nail, or mucosa of the subject. In some embodiments, the composition comprises a carrier and a tetracycline antibiotic. In some embodiments, the composition comprises a carrier, a tetracycline antibiotic, and an additional active agent. In some embodiments, the composition comprises a propellant and a foamable composition comprising a carrier and a tetracycline antibiotic. In some embodiments, the composition comprises a propellant and a foamable composition comprising a carrier, a tetracycline antibiotic, and an additional active agent. In one or more embodiments, the carrier comprises a hydrophobic solvent. In one or more embodiments the carrier is an emollient. In one or more embodiments the carrier is a hydrophobic emollient. In one or more embodiments, the carrier comprises a hydrophobic solvent and a fatty acid or a fatty alcohol or a combination thereof. In one or more embodiments, the carrier comprises a hydrophobic solvent and a wax. In one or more embodiments, the carrier comprises a hydrophobic solvent, a wax, and a fatty acid or a fatty alcohol or a combination thereof. In one or more embodiments, the composition comprises a fatty acid and/or a fatty alcohol, a wax, a tetracycline antibiotic, an additional active agent, and a hydrophobic solvent. In one or more embodiments, the carrier comprises a fatty acid or a fatty alcohol or a combination thereof. In one or more embodiments, the carrier comprises a wax. In one or more embodiments, the carrier comprises a wax, and a fatty acid or a fatty alcohol or a combination thereof. In one or more embodiments, the composition comprises a fatty acid or a fatty alcohol or a combination thereof, a wax, a tetracycline antibiotic, and an additional active agent. In some embodiments at least one fatty alcohol is a liquid at room temperature. In some embodiments at least one fatty acid is a liquid at room temperature. In some embodiments at least one wax is a liquid at room temperature. In some embodiments, the composition is a gel. In some embodiments, the composition is an ointment. In some embodiments, the composition is a foamable composition. In some embodiments, the composition is a foam. In some embodiments, the composition is a spray. In some embodiments, the fatty acid is a solid at room temperature. In some embodiments, the fatty alcohol is a solid at room temperature. In some embodiments, the wax is a solid at room temperature. In some embodiments, the fatty acid or fatty alcohol or wax is saturated. In some embodiments, the fatty acid or fatty alcohol or wax is unsaturated. In some embodiments, the fatty acid or fatty alcohol or wax is linear. In some embodiments, the fatty acid or fatty alcohol or wax is branched.

In one or more embodiments, the composition is substantially free of a fatty acid or of a fatty alcohol or of a wax or any two thereof. In one or more embodiments, the composition is essentially free of a fatty acid or of a fatty alcohol or of a wax or any two thereof. In one or more embodiments, the composition is free of a fatty acid or of a fatty alcohol or of a wax or any two thereof.

In one or more embodiments, there is provided a method for preventing or treating a drug-induced dermatose comprising a non-follicular rash in a subject, the method comprising topically administering a composition comprising a tetracycline antibiotic, for a period of at least 5 weeks, to at least a portion of the skin, nails, or mucosa of the subject prior to and/or during systemic administration of the drug to the patient, wherein the drug is selected from the group consisting of cetuximab, panitumumab, necitumumab, zalutumumab, mAb 806, mAb ICR63, mAb ICR80, mAb 225, nimotuzumab, matuzumab, erlotinib, gefitinib, lapatinib, afatinib, imatinib, nilotinib, bosutinib, ponatinib, Bcr-Abl tyrosine kinase inhibitor, sunitinib, dasatinib, canertinib, afatanib, vandetanib, and mixtures of any two or more thereof.

In one or more embodiments, there is provided a method for preventing or treating a drug-induced dermatose comprising a non-follicular rash in a subject, the method comprising topically administering a composition comprising a tetracycline antibiotic, for a period of at least 5 weeks, to at least a portion of the skin, nails, or mucosa of the subject prior to and/or during systemic administration of the drug to the patient, wherein the drug is selected from the group consisting of cetuximab, panitumumab, zalutumumab, nimotuzumab, necitumumab, matuzumab, erlotinib, gefitinib, lapatinib, canertinib, vandetanib, and mixtures of any two or more thereof.

In an embodiment, there is provided a method for preventing or treating an EGFR inhibitor induced adverse effect of the skin, nails, or mucosal membranes in a patient in need thereof, the method comprising administering a topical formulation of a tetracycline antibiotic to at least a portion of the adversely affected area; wherein the adverse effect is selected from the group consisting of skin rash; skin redness; skin dryness; nail infection; cracking, swelling, or sores of the lips or corners of the mouth; dermatitis acneiform; itchy skin; stomatitis; and paronychia.

Provided herein is a method for preventing, protecting, ameliorating, retarding, alleviating, arresting, or reversing the progression of an EGFR inhibitor induced skin or mucosal disorder in a subject, comprising topically administering, prior to and/or during a treatment regimen including systemic administration of the EGFR inhibitor, a hydrophobic composition comprising a tetracycline antibiotic to a target area on the skin or mucosa that is susceptible to developing the disorder.

Provided herein is a method for reducing the risk of introducing changes in an oncological treatment regimen that may lower the chances of success of the regimen when administered to a subject diagnosed with an internal cancer, the regimen involving the systemic administration of an EGFR inhibitor, the method comprising administering topically, prior to and/or during EGFR inhibitor administration, a hydrophobic composition comprising a tetracycline antibiotic to a target area on skin or mucosa that is susceptible to developing a disorder induced by the EGFR inhibitor.

Provided herein is a method for preventing, protecting, ameliorating, retarding, alleviating, arresting, or reversing the progression of a drug-induced dermatose comprising a non-follicular rash in a subject having an internal cancer, comprising administering topically, for a period of at least 5 weeks, to a target area on skin or mucosa that is susceptible to developing or having a drug induced dermatose, a hydrophobic composition comprising a tetracycline antibiotic, wherein the drug is selected from the group consisting of cetuximab, panitumumab, zalutumumab, nimotuzumab, necitumumab, matuzumab, erlotinib, gefitinib, lapatinib, canertinib, vandetanib and mixtures of any two or more thereof, and wherein a part of the period is prior to the application of the drug.

In one or more embodiments, the drug inducing the dermatose or side effect is a tyrosine kinase inhibitor.

In one or more embodiments, the drug-induced dermatose or side effect is selected from the group consisting of a rash, a rash unrelated to the follicular unit, a papulopustular rash, pain derived from rash, pruritus, and a pruritic rash.

In one or more embodiments, the drug-induced dermatose or side effect is pain, such as pain derived from a burn or pain derived from a wound. In some embodiments, the burn or wound is due to radiation. In some embodiments, the burn or wound is due to chemical exposure or therapy. In some embodiments, the burn or wound is due to chemical poisoning or from heat or cold. In one or more embodiments, the drug-induced dermatose or side effect is a burning or heat sensation, such as pain associated with or derived from a burn.

In one or more embodiments, the inhibitor is one or more of the inhibitors listed below:

| EGFR inhibitor monoclonal | |
|---|---|
| antibody | EGFR inhibitor tyrosine kinase |
| cetuximab | Erlotinib |
| panitumumab | Gefitinib |
| zalutumumab | Lapatinib |
| nimotuzumab | Canertinib |
| matuzumab | Vandetanib |
| mAb 806 | Imatinib |
| mAb ICR63 (CR80) | Nilotinib |
| mAb225 | bosutinib |
| bevacizumab | ponatinib |
| edrecolomab | Bcr-Abl tyrosine kinase inhibitor |
| rituximab | Sunitinib |
| trastuzumab | Dasatinib |
| | Afatanib |

In one or more embodiments, the inhibitor is a derivative of an inhibitor listed in the above table.

In an embodiment, topical administration of a hydrophobic composition comprising a tetracycline antibiotic, such as doxycycline or minocycline, twice daily provides effective drug delivery to an infected lesion site, leading to reduction in the EGFRI associated rash within only five weeks of treatment.

In an embodiment, the composition is a foamable composition. In an embodiment, the composition is presented as a foam. In one or more embodiments, the foam is a breakable foam. In another embodiment it is presented as a gel. In some embodiments, the gel is liquid; in some embodiments the gel is semi-solid. In some embodiments, the gel is stable, e.g., such that if inverted it generally maintains its shape. In one or more embodiments, when a mechanical or shear force is applied to the gel, it becomes flowable or liquid. In an embodiment, the composition is presented as an ointment. In an embodiment, the composition comprises petrolatum.

In an embodiment, the compositions are able to reduce the symptoms and severity of EGFRI associated rash. In an embodiment, improvement is apparent as the restoration of visible, normal cutaneous topographic features, indicating the return of skin integrity.

In an embodiment, no systemic side effects or no significant side effects associated with administration of the topical composition are noted. In some embodiments, there are no side effects typically associated with systemic administration of a tetracycline antibiotic. In one embodiment, the topical composition comprises an active pharmaceutical ingredient. In another embodiment the topical composition is a placebo composition. In an embodiment, dermal adverse events associated with oral tetracycline antibiotics such as oral minocycline treatment or oral doxycycline treatment are not observed or no significant side effects are noted. In an embodiment, any side effects are transitory, i.e., the side effects are substantially resolved, almost completely resolved or completely resolved after about one day to about 8 weeks of treatment, or after about 1 week to about 7 weeks of treatment, or after about 2 weeks to about 6 weeks of treatment, or after about 2 weeks to about 4 weeks of treatment, or after about 3 weeks to about 5 weeks of treatment, or after about 14 days, or after about 15 days, or after about 16 days, or after about 17 days, or after about 18 days, or after about 19 days, or after about 20 days, or after about 21 days, or after about 22 days, or after about 23 days, or after about 24 days, or after about 25 days, or after about 26 days, or after about 27 days, or after about 28 days, or after about 29 days, or after about 30 days or after about 35 days of treatment. In an embodiment, side effects or dermal adverse events (for example, pigmentation, erythema, peeling, itching and dryness) associated with known topical formulations are not observed or no significant side effects are noted. In one or more embodiments, topical application of a foamable composition comprising a tetracycline antibiotic such as doxycycline can help avoid or ameliorate side effects of EGFRI treatments, and can act to prevent or minimize such side effects, thereby leading to better patient compliance compared to available treatment options.

In one or more embodiments, topical application of a foamable composition comprising a tetracycline antibiotic such as doxycycline can help avoid or ameliorate side effects of EGFRI-associated rash, which, for example, may be generated upon use of EGFRIs with other pharmaceuticals, or treatments or may be generated upon use of EGFRIs alongside exposure to radiation and may act to prevent or minimize such side effects, thereby leading to better patient compliance compared to available treatment options.

Radiation therapy is used to treat cancer. A skin reaction sometimes called radiation dermatitis is a common side effect of radiation therapy for underlying cancer. It is a side effect of external beam ionizing radiation and the unwanted skin reaction is also referred to as radiodermatitis, x-ray dermatitis, radiation skin damage or a radiation burn. Radiation dermatitis can range from a mild rash to severe ulceration. Patients treated with radiation therapy are likely to experience a skin reaction, which in most cases can be moderate-to-severe. Local infection can also occur. Radiation therapy of underlying cancer through the skin leads to a complex pattern of skin tissue injury and an inflammatory response. Radiation dermatitis can appear within a few days to weeks after the start of radiotherapy. Its onset varies depending on the dose and frequency of the radiation coupled with the sensitivity of each patient. Skin changes are seen in areas of skin that have been irradiated, ranging from faint erythema (redness) and desquamation (itchy, peeling skin) to more severe moist desquamation (open wound), to skin necrosis (death of skin cells) and ulceration.

Acute radiation dermatitis can be graded:

Grade 1—Faint erythema or desquamation.

Grade 2—Moderate to brisk erythema or patchy, moist desquamation confined to skin folds and creases. Moderate swelling.

Grade 3—Confluent, moist desquamation greater than 1.5 cm diameter, which is not confined to the skin folds. Pitting oedema (severe swelling).

Grade 4—Skin necrosis or ulceration of full thickness dermis (middle layer of skin).

Chronic radiation dermatitis is an extension of the acute process and involves further inflammatory changes in the skin including disappearance of follicular structures (pores), increase in collagen and damage to elastic fibres in the dermis, fragile surface skin (epidermis) and telangiectasia (prominent blood vessels)

It is uncertain whether topical corticosteroids are of benefit on their own in treating radiation dermatitis although some say they can reduce the severity of the reaction.

In one or more embodiments there is provided a composition that omits topical skin irritants, such as perfumes, deodorants, short chain alcohols, surfactants and other known skin and mucosal irritants

Other unwanted side effects of the skin reaction include pain, itchiness, burning, interference with quality of life, toxicities, and reduced patient compliance.

Skin reactions may occur within 1-4 weeks of beginning radiation start, can continue during radiation therapy, and may need 2-4 weeks or more post completion to heal.

In one or more embodiments, topical application of a foamable composition comprising a tetracycline antibiotic such as doxycycline can help avoid or ameliorate the side effects of radiation therapy associated rash. In some embodiments, radiation dermatitis is generated upon use of radiation therapy alone. In some embodiments, radiation dermatitis is generated upon use of radiation therapy together with with pharmaceuticals, and topical application of a foamable composition comprising a tetracycline antibiotic such as doxycycline may act to prevent, reduce or minimize such side effects, thereby leading to better patient compliance compared to available treatment options.

In one or more embodiments, topical application of a foamable composition comprising a tetracycline antibiotic is effective in reducing the side effects by one grade. In some embodiments, it is effective in reducing the side effects by two grades. In some embodiments, it is effective in reducing the side effects by three grades. In some embodiments, it is effective in resolving the side effects. In some embodiments, it is effective after applying daily for 1 week, or for two weeks or for three weeks or for four weeks or for five weeks or for six weeks or for seven weeks or for eight weeks. In one or more embodiments, it is applied twice daily, or thrice daily, or four times daily, or five times daily or six times daily, instead of once daily. In some embodiments, it is applied during radiation therapy. In some embodiments it is applied before (e.g., two weeks, or one week, or several says to one day before radiation therapy) as well as during radiotherapy. In some embodiments it is applied during and after radiotherapy (e.g., four weeks, or three weeks, or two weeks, or one week or several days to one day after radiation therapy. In some embodiments it is applied before, during and after radiotherapy. In one or more embodiments, topical application of a foamable composition comprising a tetracycline antibiotic is applied according to any of the methods, regimes, frequency and amounts provided or described herein with respect to treatments of EGFRI associated rash. In some embodiments, topical application of a foamable composition comprising a tetracycline antibiotic and another active agent is applied during radiotherapy, or before and during or before and during an after radiotherapy as provided and described herein.

In one or more embodiments, topical application of a foamable composition comprising a tetracycline antibiotic such as doxycycline can help avoid or ameliorate side effects of other treatments that include EGFRI-associated rash, e.g., generated upon use of EGFRIs with other pharmaceuticals, or alongside exposure to radiation therapy or both and may act to prevent or minimize such side effects, thereby leading to better patient compliance compared to available treatment options.

In one or more embodiments, the topical composition can comprise two or more tetracycline antibiotics, for example can comprise minocycline and doxycycline. In one or more embodiments, the topical composition can comprise a tetracycline antibiotic and a second active pharmaceutical agent.

In one or more embodiments, there is provided a method of preventing, treating or alleviating a disorder selected from the group consisting of EGFRI associated rash, EGFRI associated rash related symptoms, a sebaceous gland disorder, EGFRI associated rash bacteria associated disorders, and other superficial skin or mucosal disorders that are a by-product of a therapeutic treatment or treatment regime applied to a subject, including other pharmaceutical active agents and radiation therapy, comprising administering topically at least once daily to a target area on a human subject having the disorder a hydrophobic gel or foam composition comprising a tetracycline antibiotic, wherein the target area is the skin.

In one or more embodiments, there is provided a method of preventing, protecting, ameliorating, retarding, alleviating or treating a drug-induced dermatose, comprising administering a topical composition comprising a tetracycline antibiotic.

In one or more embodiments, there is provided a topical composition comprising a tetracycline antibiotic for use in preventing, protecting, ameliorating, retarding, alleviating or treating a drug-induced dermatose.

In one or more embodiments, there is provided a use of a topical composition comprising a tetracycline antibiotic for the manufacture of a medicament for preventing, alleviating, protecting, ameliorating, retarding or treating a drug-induced dermatose.

In one or more embodiments, the induced dermatose is a rash, an EGFR inhibitor associated rash, a papulopustular rash, a pruritic rash, exanthemas, morbilliforms, macular eruptions, papular eruptions, dermatitis, erythema nodosum, erythema multiforme/stevens-johnson/toxic epidermal necrolysis, eczematous eruption, cutaneous necrosis, psoriasiform reaction, lichenoid reaction, EGFRI associated rashiform eruptions, bullous eruptions, pustular eruptions, acute neutrophilic dermatoses, pityriasis rosea-like eruptions, porphyria, pseudoporphyria, systemic lupus erythematosus, pseudolymphoma, alopecia, or hypertrichosis.

In one or more embodiments, the dermatose is induced by an anti-cancer treatment, a treatment with EGFR inhibitors, a treatment with tyrosine kinase inhibitors, acetaminophen, allopurinol, monoclonal antibodies, antibiotics (particularly erythromycin, penicillins, and sulfonamides), barbiturates, carbamazepine, cephalosporins, chlorpromazine, griseofulvin, insulin, metronidazole, NSAIDs (including aspirin and coxibs), paclitaxel, phenolphthalein, proteins, pseudoephedrine, rifampin, salicylates, or sulphonamides.

In one or more embodiments, the topical composition for preventing, alleviating, protecting, ameliorating, retarding or treating a drug-induced dermatose is administered once daily, twice daily or three-times daily. In one or more embodiments, the topical composition is administered before, together with, or after the onset of the EGFR inhibitor treatment. In one or more embodiments, the topical composition is administered for about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, or for more than about 12 weeks. In one or more embodiments, the topical composition for preventing, protecting, ameliorating, retarding, alleviating, or treating a drug-induced dermatose is administered as long as the drug inducing the dermatose is administered.

In one or more embodiments, the topical composition for preventing, alleviating or treating a drug-induced dermatose comprises a tetracycline antibiotic selected from the group consisting of a minocycline, a doxycycline, a tigecycline, a tetracycline, a chlortetracycline, an oxytetracycline, a demeclocycline, a methacycline, and pharmaceutically acceptable salts or hydrates thereof.

In one or more embodiments, the topical composition for preventing, alleviating or treating a drug-induced dermatose is a hydrophobic gel or foam composition comprising a therapeutically effective amount of a tetracycline antibiotic.

In one or more embodiments, the topical composition for preventing, alleviating or treating a drug-induced dermatose comprises a combination of a tetracycline antibiotic and a second active agent. In one or more embodiments, the second active agent is selected from the group consisting of steroids, corticosteroids, anti-EGFRI associated rash agents, retinoids, benzoyl peroxide, salicylic acid, non-steroidal anti-inflammatory drugs, immunomodulators, imiquimod, pimecrolimus, tacrolimus, antibiotics, penicillins, antifungals, antivirals and a mixture of any two or more thereof.

In one or more embodiments, topical administration of a tetracycline antibiotic foam provided herein to a patient under EGFR inhibitor treatment prevents or protects or reduces the outbreak of rash or EGFR inhibitor-related dermatose by about 10%, by about 20%, by about 30%, by about 40%, by about 50%, by about 60%, by about 70%, by about 80%, by about 90%, or by about 100%, as evaluated using EGFRI-associated cutaneous toxicity grade, and/or Common Terminology Criteria for Adverse Events (CTCAE) v3.0 grade for rash, and/or Erythema score, and/or Lesion counts, and/or Pain VAS marked by the subject, and/or Pruritus VAS marked by the subject Photograph of face, and/or Skindex 16 and/or percentage of face surface area involvement.

In one or more embodiments, the prevention or reduction of a rash outbreak or EGFR-inhibitor-related dermatose is achieved about 1 week, about 2 weeks, about 3 weeks, about 4 weeks, about 5 weeks, about 6 weeks, about 7 weeks, about 8 weeks, about 9 weeks, about 10 weeks, or more than about 10 weeks after the start of the tetracycline antibiotic topical treatment.

In one or more embodiments, the tetracycline composition acts by protecting or partially protecting the skin or mucosa from the effects of monoclonal antibody therapy, for example, by providing a level of protection against EGFR inhibitor associated rash and/or a drug-induced dermatose.

In one or more embodiments, there is provided a method of treating or alleviating an EGFRI associated rash. In one or more embodiments, there is provided a method of treating or alleviating EGFRI associated rash related symptoms. In one or more embodiments, there is provided a method of treating or alleviating a tetracycline antibiotic responsive EGFRI associated rash related disorder. In one or more embodiments, there is provided a method of treating or alleviating a superficial skin or mucosal disorder that is a by-product of a therapeutic treatment or treatment regime applied to a subject including EGFRI. In one or more embodiments, the EGFRI associated rash may involve skin infections or other skin conditions that are treatable with a tetracycline antibiotic. In one or more embodiments, the EGFRI associated rash may involve a skin disorder caused by a bacteria. In one or more embodiments, the EGFRI associated rash may involve a tetracycline antibiotic responsive disorder. In one or more embodiments, the EGFRI associated rash may involve a sebaceous gland disorder.

The points, positions, methods, regimes, effects of treatment etc., indicated throughout the specification in relation to EGFRI associated disorders, e.g. rash, may also similarly apply to the effects of combined treatments such as other pharmaceutical agents with EGFRIs or radiation therapy with EGFRIs, whether simultaneously, consecutively or overlapping and may display similar symptoms and responses.

In one or more embodiments, the hydrophobic gel or foam composition for use in the method comprises:
a) about 60% to about 95% by weight of at least one hydrophobic solvent or carrier;
b) at least one viscosity-modifying agent selected from the group consisting of a fatty alcohol, a fatty acid, and a wax; and
c) a therapeutically effective amount of a tetracycline antibiotic.

In one or more embodiments, the hydrophobic gel or foam composition for use in the method comprises:
a) about 60% to about 95% by weight of at least one hydrophobic solvent or carrier;
b) at least one viscosity-modifying agent selected from the group consisting of a fatty alcohol, a fatty acid, and a wax;
c) a therapeutically effective amount of a tetracycline antibiotic; and
d) an additional active agent.

In one or more embodiments, the hydrophobic foam for use in the method is formed from the hydrophobic gel composition further comprising a propellant.

In an embodiment, the disorder is EGFRI associated rash.

In an embodiment, the disorder is an inflammatory disorder.

In an embodiment, the disorder is a non-inflammatory disorder.

In one or more embodiments, the tetracycline antibiotic for use in the method is selected from the group consisting of a tetracycline, an oxytetracycline, a demeclocycline, a doxycycline, doxycycline hyclate, a lymecycline, a meclocycline, a methacycline, a minocycline, minocycline hydrochloride, a rolitetracycline, a chlorotetracycline, and a tigecycline.

In one or more embodiments, the tetracycline antibiotic for use in the method is present in the composition at a concentration of about 0.1% to about 16% by weight.

In one or more embodiments, the tetracycline antibiotic for use in the method is doxycycline hyclate or minocycline hydrochloride. In one or more embodiments, the tetracycline antibiotic, e.g., doxycycline hyclate or minocycline hydrochloride, for use in the method is present in the composition at a concentration of about 0.1% by weight, about 0.2% by weight, about 0.5% by weight, about 0.6% by weight, about 0.7% by weight, about 0.8% by weight, about 0.9% by weight, about 1% by weight, about 1.1% by weight, about 1.2% by weight, 1.3% by weight, about 1.4% by weight, about 1.5% by weight, about 2% by weight, about 2.5% by weight, about 3% by weight, about 3.5% by weight, about 4% by weight, about 4.5% by weight, about 5% by weight, about 5.5% by weight, about 6% by weight, about 6.5% by weight, about 7% by weight, about 7.5% by weight, about 8% by weight, about 8.5% by weight, about 9% by weight, about 9.5% by weight, about 10% by weight, about 10.5% by weight, about 11% by weight, about 11.5% by weight, about 12% by weight, about 12.5% by weight, about 13% by weight, about 13.5% by weight, about 14% by weight, about 14.5% by weight, or about 15% by weight about 16% by weight, or about 17% by weight about 18% by weight, or about 19% by weight about 20% by weight, or at a range between any of the aforesaid figures such as between about 0.5% by weight to about 5.5% by weight.

In one or more embodiments, the hydrophobic gel or foam composition for use in the method is applied on average at a frequency selected from the group consisting of three times daily, twice daily, once daily, and on alternate days.

In one or more embodiments, the hydrophobic gel or foam composition for use in the method is administered for a period of time selected from the group consisting of two weeks, three weeks, four weeks, five weeks, six weeks, seven weeks, eight weeks, nine weeks, ten weeks, eleven weeks, twelve weeks, thirteen weeks, fourteen weeks, fifteen weeks, sixteen weeks, or for the entire duration of EGFRI treatment.

In one or more embodiments, the hydrophobic gel or foam composition for use in the method is applied as a maintenance dose after the EGFRI therapy period at a frequency selected from the group consisting of every two days, three times a week, twice a week, once a week, once in two weeks, once in three weeks, once a month, once in two months, and alternate weeks. In one or more embodiments, the maintenance dose is discontinued after a period selected from the group consisting of a week, two weeks, three weeks, four weeks, a month, two months, three months, four months, five months, and six months.

In one or more embodiments, the hydrophobic foam composition or gel for use in the methods provided herein is effective against EGFRI associated rash.

In one or more embodiments, the hydrophobic foam composition or gel for use in the methods provided herein results in a decrease of at least about 40% in the number of the EGFRI associated rash lesions or in the area covered by the lesions or in the severity of the lesions after five weeks of treatment, when the hydrophobic foam composition or gel is administered once daily. In one or more embodiments, the decrease is at least about 30%, at least about 35%, at least about 45%, at least about 50%, at least about 55%, at least about 60%, at least about 65%, or at least about 70%.

In one or more embodiments, the hydrophobic foam composition or gel for use in the methods provided herein results in a decrease of at least about 30% in the number of the EGFRI associated rash lesions or in the area covered by the lesions or in the severity of the lesions four weeks after the end of the treatment with the hydrophobic foam composition or gel. In one or more embodiments the decrease is at least about 20%, at least about 25%, at least about 35%, at least about 40%, at least about 45%, at least about 50%, at least about 55%, at least about 60%, at least about 65%, or at least about 70%.

In one or more embodiments, the hydrophobic foam composition or gel for use in the methods provided herein results in a decrease of at least about 30% in the number of the EGFRI associated rash lesions or in the area covered by the lesions or in the severity of the lesions after five weeks of treatment, wherein the composition is administered twice daily. In one or more embodiments the decrease is at least about 20%, at least about 25%, at least about 35%, at least about 40%, at least about 45%, at least about 50%, at least about 55%, at least about 60%, at least about 65%, or at least about 70%.

In one or more embodiments, the hydrophobic foam composition or gel for use in the methods provided herein results in a decrease of at least 70% in the number of the EGFRI associated rash lesions or in the area covered by the lesions or in the severity of the lesions four weeks after the end of the treatment with the hydrophobic foam composition or gel. In one or more embodiments the decrease is at least about 30%, at least about 35%, at least about 40%, at least about 45%, at least about 50%, at least about 55%, at least about 60%, or at least about 65%.

In one or more embodiments, the hydrophobic foam composition or gel for use in the methods provided herein slows or reduces or ameliorates or prevents the development of a moderate to severe EGFRI associated rash and/or reduces or ameliorates the severity of such a rash in at least about 25% of patients undergoing treatment with one or more EGFRIs, or at least in about 30% of the patients, or at least in about 35% of the patients, or at least in about 40% of the patients, or at least in about 45% of the patients, or at least in about 50% of the patients, or at least in about 55% of the patients, or at least in about 60% of the patients, or at least in about 65% of the patients. In one or more embodiments, the amelioration, reduction or slowing is expressed by a decrease in the number of lesions/rash, or area of lesions/rash, or intensity of lesions/rash or severity of lesions/rash or density of lesions/rash in a given area e.g. face. In one or more embodiments prevention means, for example, the absence of appearance of a rash of significance or the absence of a significant worsening or deterioration in the rash. In one or more embodiments prevention is expressed by the absence of a significant increase in one or more of these parameters. In one or more embodiments reduction is expressed, for example, by a reduction in one of the above parameters in an individual or in a pool of individuals by about 5%, or about 10%, or about 15%, or about 20%, or about 25%, or about 30%, or about 35%, or about 40%, or about 45%, or about 50%, or about 55%, or about 60%, or about 65%, or more. In one or more embodiments amelioration is expressed, for example, by a change in grading, e.g. based on recognized scales, such as MESTT, or Scope Scale, of one or more of the above parameters in an individual or in a pool of individuals. Grading can include for example from severe to moderate, or from moderate to mild or from mild to normal or from severe to mild or from moderate to normal or any other such recognized clinical method of assessing a clinical trial and grading the patients. In one or more embodiments grading can be based on quality of life scales, such as QOL, or QOLS, or DLQI (Dermatology Life Quality Index). In one of one or more embodiments slowing is expressed by an increase in time before the appearance of a rash or development of a rash is seen in a given pool of individuals or in an individual. This increase in time may be about a day, or about two days, or about three days, or about four days, or about five days, or about six days, or about seven days, or about eight days, or about nine days, or about ten days, or about eleven days, or about twelve days, or about thirteen days, or about fourteen days, or about three weeks, or about four weeks, or about five weeks, or about six weeks, or about seven weeks.

In one or more embodiments, the hydrophobic foam composition or gel for use in the methods provided herein prevents the worsening or deterioration of a moderate to severe EGFRI associated rash in at least about 25% of patients undergoing treatment with one or more EGFRIs, or at least in about 30% of the patients, or at least in about 35% of the patients, or at least in about 40% of the patients, or at least in about 45% of the patients, or at least in about 50% of the patients, or at least in about 55% of the patients, or at least in about 60% of the patients or at least in about 65% of the patients.

In one or more embodiments, the hydrophobic foam composition or gel for use in the methods provided herein slows or reduces or ameliorates or prevents the development of a severe EGFRI associated rash (grade 3) in about 25% of the patients, or at least in about 30% of the patients, about 35% of the patients, about 40% of the patients, about 45% of the patients, about 50% of the patients, about 55% of the patients, about 60% of the patients, about 65% of the patients.

In one or more embodiments, the hazard ratio of developing a more severe rash when on the placebo (vehicle) is about 0.2, which indicated a higher possibility of developing a grade 3 rash when administered the vehicle as compared to the compositions comprising the hydrophobic foam compositions or gel described herein. In some embodiments, the hazard ratio of developing a more severe rash when on the placebo (vehicle) is about 0.3, or about 0.4, or about 0.5.

In one or more embodiments, the odds of developing a more severe rash when on the placebo are about up to or more than 2 times higher, or about up to or more than 3 times higher, or about up to or more than 4 times higher, or about up to or more than 5 times higher, or about up to or more than 6 times higher, or about up to or more than 7 times higher, or about up to or more than 8 times higher, or about up to or more than 9 times higher, or about up to or more than 10 times higher than developing such a rash when on the hydrophobic foam composition or gel for use in the methods provided herein.

In one or more embodiments, the odds of developing a more severe rash when on the hydrophobic foam composition or gel for use in the methods provided herein are about 2 times lower, or about 3 times lower, or about 4 times lower, or about 5 times lower, or about 6 times lower, or about 7 times lower, or about 8 times lower, or about 9 times lower, or about 10 times lower than developing such a rash when on the placebo.

In one or more embodiments, the hydrophobic foam composition or gel for use in the methods provided herein provides a lower severity score in an individual when comparing a treated area to a comparable non treated area or in a pool of individuals a lower mean and/or median severity scores compared with the placebo.

In one or more embodiments, the hydrophobic gel or foam composition for use in the methods provided herein comprises:

about 48% to about 51% by weight of soybean oil;
about 23% to about 25% by weight of coconut oil;
about 4% to about 6% by weight of cyclomethicone;
about 3.2% to about 5.5% by weight of light mineral oil;
about 3% to about 4% by weight of cetostearyl alcohol;
about 2% to about 4% by weight of stearic acid;
about 2% to about 3% by weight of myristyl alcohol;
about 1% to about 3% by weight of hydrogenated castor oil;
about 1% to about 3% by weight of beeswax;
about 1% to about 2% by weight of stearyl alcohol;
about 0.5% to about 1.5% by weight of behenyl alcohol; and
about 1% by weight of minocycline hydrochloride or doxycycline hyclate.

In one or more embodiments, the hydrophobic gel or foam composition used in the methods provided herein comprises:

about 48% to about 51% by weight of soybean oil;
about 23% to about 25% by weight of coconut oil;
about 4% to about 6% by weight of cyclomethicone;
about 0.7% to about 2% by weight of light mineral oil;
about 3% to about 4% by weight of cetostearyl alcohol;
about 2% to about 4% by weight of stearic acid;
about 2% to about 3% by weight of myristyl alcohol;
about 1% to about 3% by weight of hydrogenated castor oil;
about 1% to about 3% by weight of beeswax;
about 1% to about 2% by weight of stearyl alcohol;
about 0.5% to about 1.5% by weight of behenyl alcohol; and
about 4% by weight of minocycline hydrochloride or doxycycline hyclate.

In one or more embodiments, the hydrophobic gel or foam composition used in the methods provided herein comprises:

about 57.6% to about 87.5% by weight of heavy mineral oil;
about 3.5% to about 6.5% by weight of light mineral oil;
about 3.2% to about 5.9% by weight of stearyl alcohol;
about 1.75% to about 3.25% by weight of stearic acid;
about 0.8% to about 1.4% by weight of behenyl alcohol; and
about 3.3% to about 6.1% by weight of minocycline hydrochloride or doxycycline hyclate.

In one or more embodiments, the hydrophobic gel or foam composition used in the methods provided herein comprises:

about 65.8% to about 86% by weight of heavy mineral oil;
about 4% to about 6% by weight of light mineral oil;
about 3.6% to about 5.4% by weight of stearyl alcohol;
about 2% to about 3% by weight of stearic acid;
about 0.9% to about 1.3% by weight of behenyl alcohol; and
about 3.7% to about 5.6% by weight of minocycline hydrochloride or doxycycline hyclate.

In one or more embodiments, the hydrophobic gel or foam composition used in the methods provided herein comprises:

about 74% to about 84% by weight of heavy mineral oil;
about 4.5% to about 5.5% by weight of light mineral oil;
about 4.1% to about 5% by weight of stearyl alcohol;
about 2.3% to about 2.8% by weight of stearic acid;
about 1% to about 1.2% by weight of behenyl alcohol; and
about 4.2% to about 5.1% by weight of minocycline hydrochloride or doxycycline hyclate.

In one or more embodiments, the hydrophobic gel or foam composition used in the methods provided herein comprises:

about 82.24% by weight of heavy mineral oil;
about 5% by weight of light mineral oil;
about 4.5% by weight of stearyl alcohol;
about 2.5% by weight of stearic acid;
about 1.1% by weight of behenyl alcohol; and
about 4.66% by weight of minocycline hydrochloride or doxycycline hyclate.

In one or more embodiments, the hydrophobic gel or foam composition used in the methods provided herein comprises:

about 62% to about 91.7% by weight of heavy mineral oil, light mineral oil or combinations thereof;
about 2.6% to about 4.8% by weight of stearyl alcohol;
about 1.75% to about 3.25% by weight of stearic acid;
about 0.5% to about 0.9% by weight of behenyl alcohol;
about 0.14% to about 0.26% by weight of paraffin 51-53; and
about 3.3% to about 6.1% by weight of minocycline hydrochloride or doxycycline hyclate.

In one or more embodiments, the hydrophobic gel or foam composition used in the methods provided herein comprises:

about 70.6% to about 90.6% by weight of heavy mineral oil, light mineral oil or combinations thereof;
about 3% to about 4.4% by weight of stearyl alcohol;
about 2% to about 3% by weight of stearic acid;
about 0.56% to about 0.84% by weight of behenyl alcohol;
about 0.16% to about 0.24% by weight of paraffin 51-53; and
about 3.7% to about 5.6% by weight of minocycline hydrochloride or doxycycline hyclate.

In one or more embodiments, the hydrophobic gel or foam composition used in the methods provided herein comprises:

about 79.4% to about 89.4% by weight of heavy mineral oil, light mineral oil or combinations thereof;
about 3.3% to about 4.1% by weight of stearyl alcohol;
about 2.3% to about 2.8% by weight of stearic acid;
about 0.63% to about 0.77% by weight of behenyl alcohol;
about 0.18% to about 0.22% by weight of paraffin 51-53; and
about 4.2% to about 5.6% by weight of minocycline hydrochloride or doxycycline hyclate.

In one or more embodiments, the hydrophobic gel or foam composition used in the methods provided herein comprises:

about 63% to about 98% by weight of heavy mineral oil;
about 0.1% to about 15% by weight of light mineral oil;
about 0.5% to about 7% by weight of stearyl alcohol;
about 0.5% to about 5% by weight of stearic acid;
about 0.2% to about 2% by weight of behenyl alcohol; and
about 1% to about 8% by weight of minocycline hydrochloride or doxycycline hyclate.

In one or more embodiments, the hydrophobic gel or foam composition used in the methods provided herein comprises:

about 73% to about 98% by weight of heavy mineral oil, light mineral oil or combinations thereof;
about 0.5% to about 7% by weight of stearyl alcohol;
about 0.5% to about 5% by weight of stearic acid;
about 0.2% to about 2% by weight of behenyl alcohol;
about 0.1% to about 5% by weight of paraffin 51-53; and
about 1% to about 8% by weight of minocycline hydrochloride or doxycycline hyclate.

In one or more embodiments, the fatty alcohol is about 0.1% to about 10% by weight or about 0.2% to about 9% by weight or about 0.3% to about 8% by weight or about 0.4% to about 7% by weight or about 0.5% to about 6% by weight or about 0.6% to about 5% by weight or about 0.7% to about 4% by weight or about 0.8% to about 3% by weight or about 0.9% to about 2% by weight or any range between any of the aforesaid numbers, such as about 0.2% to about 7% by weight or about 0.1% to about 0.9% by weight. In one or more embodiments, the fatty acid is about 0.1% to about 10% by weight or about 0.2% to about 9% by weight or about 0.3% to about 8% by weight or about 0.4% to about 7% by weight or about 0.5% to about 6% by weight or about 0.6% to about 5% by weight or about 0.7% to about 4% by weight or about 0.8% to about 3% by weight or about 0.9% to about 2% by weight or any range between any of the aforesaid numbers, such as about 0.2% to about 7% by weight or about 0.1% to about 0.9% by weight. In one or more embodiments, the wax is about 0.1% to about 10% by weight or about 0.2% to about 9% by weight or about 0.3% to about 8% by weight or about 0.4% to about 7% by weight or about 0.5% to about 6% by weight or about 0.6% to about 5% by weight or about 0.7% to about 4% by weight or about 0.8% to about 3% by weight or about 0.9% to about 2% by weight or any range between any of the aforesaid numbers, such as about 0.2% to about 7% by weight or about 0.1% to about 0.9% by weight. In some embodiments, the solid wax and solid fatty alcohol or the solid wax and solid fatty acid is about 4% to about 15% by weight or about 5% to about 14% by weight or about 6% to about 13% by weight or about 7% to about 12% by weight or about 8% to about 11% by weight or about 9% to about 10% by weight or any range between any of the aforesaid numbers, such as about 4% to about 7% by weight or about 5% to about 12% by weight. In one or more embodiments, the hydrophobic gel or foam composition used in the methods provided herein comprises a total amount of solid components of about 4% to about 15% by weight. In one or more embodiments, the hydrophobic gel or foam composition used in the methods provided herein comprises stearyl alcohol, stearic acid, behenyl alcohol, paraffin 51-53, or mixtures of any two or more thereof, in a total amount of about 4% to about 15% by weight.

In one or more embodiments, the ratio between the hydrophobic solvent and the fatty alcohol is about 100:1, or about 90:1, or about 80:1, or about 70:1, or about 60:1, or about 50:1, or about 40:1, or about 30:1, or about 20:1, or about 10:1, or about 9:1, or about 8:1, or about 7:1, or about 6:1, or about 5:1, or about 4:1, or about 3:1, or about 2:1, or about 1:1, or about 1:2, or about 1:3, or about 1:4, or about 1:5, or about 1:6, or about 1:7, or about 1:8, or about 1:9, or about 1:10, or about 1:20, or about 1:30, or about 1:40, or about 1:50, or about 1:60, or about 1:70, or about 1:80, or about 1:90, or about 1:100, or about 15:1, or about 16:1, or about 17:1, or about 19:1, or about 20:1, or about 21:1, or about 22:1, or about 15.6:1, or about 21.6:1, or about 19.5:1, or about 16.5:1.

In one or more embodiments, the ratio between the hydrophobic solvent and the fatty acid is about 100:1, or about 90:1, or about 80:1, or about 70:1, or about 60:1, or about 50:1, or about 40:1, or about 30:1, or about 20:1, or about 10:1, or about 9:1, or about 8:1, or about 7:1, or about 6:1, or about 5:1, or about 4:1, or about 3:1, or about 2:1, or about 1:1, or about 1:2, or about 1:3, or about 1:4, or about 1:5, or about 1:6, or about 1:7, or about 1:8, or about 1:9, or about 1:10, or about 1:20, or about 1:30, or about 1:40, or about 1:50, or about 1:60, or about 1:70, or about 1:80, or about 1:90, or about 1:100, or about 29:1, or about 31:1, or about 32:1, or about 33:1, or about 34:1, or about 35:1, or about 36:1 or about 37:1, or about 38:1, or about 39:1, or about 41:1, or about 42:1, or about 43:1, or about 44:1, or about 35:1, or about 35.3:1, or about 34.8:1, or about 37:1, or about 36.6:1, or about 33:1.

In one or more embodiments, the ratio between the hydrophobic solvent and the total amount of fatty alcohol and fatty acid is about 100:1, or about 90:1, or about 80:1, or about 70:1, or about 60:1, or about 50:1, or about 40:1, or about 30:1, or about 20:1, or about 19:1, or about 18:1, or about 17:1, or about 16:1, or about 15:1, or about 14:1, or about 13:1, or about 12:1, or about 10:1, or about 9:1, or about 8:1, or about 7:1, or about 6:1, or about 5:1, or about 4:1, or about 3:1, or about 2:1, or about 1:1, or about 1:2, or about 1:3, or about 1:4, or about 1:5, or about 1:6, or about 1:7, or about 1:8, or about 1:9, or about 1:10, or about 1:11, or about 1:12, or about 1:13, or about 1:14, or about 1:15, or about 1:16, or about 1:17, or about 1:18, or about 1:19, or about 1:20, or about 1:30, or about 1:40, or about 1:50, or about 1:60, or about 1:70, or about 1:80, or about 1:90, or about 1:100, or about 10.8:1, or about 12.8:1, or about 10.7:1, or about 13.6:1, or about 11:1.

In one or more embodiments, the hydrophobic gel or foam composition used in the methods provided herein is substantially free of one or more of soybean oil, coconut oil, cyclomethicone, cetostearyl alcohol, myristyl alcohol, beeswax, and hydrogenated castor oil.

In one or more embodiments, the hydrophobic gel or foam composition used in the methods provided herein is essentially free of one or more of soybean oil, coconut oil, cyclomethicone, cetostearyl alcohol, myristyl alcohol, beeswax, and hydrogenated castor oil.

In one or more embodiments, the hydrophobic gel or foam composition used in the methods provided herein is free of one or more of soybean oil, coconut oil, cyclomethicone, cetostearyl alcohol, myristyl alcohol, beeswax, and hydrogenated castor oil.

In one or more embodiments, the hydrophobic gel or foam composition used in the methods provided herein comprises:

about 81.94% by weight of heavy mineral oil;
about 5% by weight of light mineral oil;
about 4.5% by weight of stearyl alcohol;
about 2.5% by weight of stearic acid;
about 1.1% by weight of behenyl alcohol;
about 4.66% by weight of minocycline hydrochloride or doxycycline hyclate; and
about 0.3% by weight of adapalene.

In one or more embodiments, the hydrophobic gel or foam composition used in the methods provided herein comprises:

about 82% by weight of heavy mineral oil;
about 5% by weight of light mineral oil;
about 4.5% by weight of stearyl alcohol;
about 2.5% by weight of stearic acid;
about 1.1% by weight of behenyl alcohol;
about 4.8% by weight of minocycline hydrochloride or doxycycline hyclate; and
about 0.1% by weight of adapalene.

In one or more embodiments, the hydrophobic gel or foam composition used in the methods provided herein comprises:

about 88.6% by weight of heavy mineral oil;
about 3.6% by weight of stearyl alcohol;
about 2.4% by weight of stearic acid;
about 0.5% by weight of behenyl alcohol;
about 4.8% by weight of minocycline hydrochloride or doxycycline hyclate; and
about 0.1% by weight of adapalene.

In one or more embodiments, the hydrophobic gel or foam composition used in the methods provided herein comprises:

about 50% by weight of soybean oil;
about 23.6% by weight of coconut oil;
about 5% by weight of cyclomethicone;
about 0.7% by weight of light mineral oil;
about 3.5% by weight of cetostearyl alcohol;
about 3% by weight of stearic acid;
about 2.5% by weight of myristyl alcohol;
about 2% by weight of hydrogenated castor oil;
about 2% by weight of beeswax;
about 1.5% by weight of stearyl alcohol;
about 1.1% by weight of behenyl alcohol;
about 4.8% by weight of minocycline hydrochloride or doxycycline hyclate; and
about 0.3% by weight of adapalene.

In one or more embodiments, the hydrophobic gel or foam composition used in the methods provided herein comprises:

about 49% by weight of heavy mineral oil;
about 39% by weight of light mineral oil;
about 3.8% by weight of stearyl alcohol;
about 2.4% by weight of stearic acid;
about 0.7% by weight of behenyl alcohol;
about 4.8% by weight of minocycline hydrochloride or doxycycline hyclate; and
about 0.3% by weight of adapalene.

In one or more embodiments, the hydrophobic gel or foam composition used in the methods provided herein comprises:

about 43.4% by weight of heavy mineral oil;
about 39% by weight of light mineral oil;
about 4.3% by weight of stearyl alcohol;
about 2.5% by weight of stearic acid;
about 5% by weight of cyclomethicone;
about 0.7% by weight of behenyl alcohol;
about 4.8% by weight of minocycline hydrochloride or doxycycline hyclate; and
about 0.3% by weight of adapalene.

In one or more embodiments, other suitable doses of tetracycline antibiotics are incorporated and the amount by weight of one or more oils is adjusted so the composition prior to addition of propellant is 100% as would be appreciated by one skilled in the art.

In one or more embodiments, the hydrophobic foam for use in the method is formed from the hydrophobic gel composition and further comprises about 3% to about 25% by weight of propellant based on the total weight of the hydrophobic gel composition.

In one or more embodiments, pre-emptive treatment with the hydrophobic foam composition or gel provided herein results in a decrease in incidence of grade 2 skin rash by more than 50% compared to placebo without additional side effects after five weeks or less than five weeks of treatment with the hydrophobic foam composition or gel, wherein the composition is administered twice daily.

In one or more embodiments, pre-emptive treatment with the hydrophobic foam composition or gel provided herein results in a decrease of at least one grade in rash severity in an individual. In one or more embodiments, an individual patient receiving EGFRI who develops a moderate to severe rash will experience a decrease in grade after five weeks or in less than five weeks, or after four weeks or in less than four weeks, or after three weeks or in less than three weeks, or after two weeks or in less than two weeks of treatment with the hydrophobic foam composition or gel, wherein the composition is administered twice daily. In one or more embodiments in a pool of individuals undergoing pre-emptive treatment, there is a decrease of at least one grade in at least about 30%, or at least about 35%, or at least about 40%, or at least about 45%, or at least about 50%, or at least about 55%, or at least about 60% of the patients receiving EGFRI who develop a moderate to severe rash, after five weeks or less than five weeks of treatment or after four weeks or in less than four weeks, or after three weeks or in less than three weeks, or after two weeks or in less than two weeks with the hydrophobic foam composition or gel, wherein the composition is administered twice daily.

In one or more embodiments, pre-emptive treatment with the hydrophobic foam composition or gel provided herein results in a decrease of at least one grade in rash severity in an individual. In one or more embodiments, an individual patient receiving EGFRI who develops a severe rash will experience a decrease in grade after five weeks or in less than five weeks, or after four weeks or in less than four weeks, or after three weeks or in less than three weeks, or after two weeks or in less than two weeks of treatment with the hydrophobic foam composition or gel, wherein the composition is administered twice daily. In one or more embodiments in a pool of individuals undergoing pre-emptive treatment, there is a decrease of at least one grade, in at least about 25%, in at least about 30%, at least about 35%, at least about 40%, at least about 45%, at least about 50%, at least about 55%, at least about 60%, at least about 65%, at least about 70%, at least about 75%, at least about 80% of the patients receiving EGFRI who develop a severe rash, after five weeks or less than five weeks or after four weeks or in less than four weeks, or after three weeks or in less than three weeks, or after two weeks or in less than two weeks of treatment with the hydrophobic foam composition or gel, wherein the composition is administered twice daily.

In one or more embodiments, pre-emptive treatment with the hydrophobic foam composition or gel provided herein results in a decrease of at least two grades in rash severity in an individual. In one or more embodiments, an individual patient receiving EGFRI who develops a moderate to severe rash will experience a decrease in two grades after five weeks or in less than five weeks, or after four weeks or in less than four weeks, or after three weeks or in less than three weeks, or after two weeks or in less than two weeks of treatment with the hydrophobic foam composition or gel, wherein the composition is administered twice daily. In one or more embodiments in a pool of individuals undergoing pre-emptive treatment, there is a decrease of at least two grades in about 20%, or in about 25%, or in about 30% or more of the patients receiving EGFRI who develop a moderate to severe rash, after five weeks or less than five weeks or after four weeks or in less than four weeks, or after three weeks or in less than three weeks, or after two weeks or in less than two weeks of treatment with the hydrophobic foam composition or gel, wherein the composition is administered twice daily.

In one or more embodiments, pre-emptive treatment with the hydrophobic foam composition or gel provided herein results in a decrease of at least two grades in rash severity in an individual. In one or more embodiments, an individual patient receiving EGFRI who develops a severe rash will experience a decrease in at least two grades after five weeks or in less than five weeks, or after four weeks or in less than four weeks, or after three weeks or in less than three weeks, or after two weeks or in less than two weeks of treatment with the hydrophobic foam composition or gel, wherein the composition is administered twice daily. In one or more embodiments in a pool of individuals undergoing pre-emptive treatment, there is a decrease of at least two grades in about 20%, or in about 25%, or in about 30%, or in about 35%, or in about 40% or more of the patients receiving EGFRI who develop a severe rash, after five weeks or less than five weeks or after four weeks or in less than four weeks, or after three weeks or in less than three weeks, or after two weeks or in less than two weeks of treatment with the hydrophobic foam composition or gel, wherein the composition is administered twice daily.

In one or more embodiments, the pre-emptive treatment with the hydrophobic foam composition or gel provided herein results in a decrease of at least one grade in rash severity in an individual. In one or more embodiments, an individual patient receiving EGFRI who develops a moderate to severe rash will experience or maintain a decrease in grade four weeks after the end of the treatment with the hydrophobic foam composition or gel, wherein the composition is administered twice daily for a period of about eight weeks or for about seven weeks, or for about six weeks, or for about five weeks, or for about four weeks, or for about three weeks, or for about two weeks. In one or more embodiments in a pool of individuals undergoing pre-emptive treatment, there is a decrease of at least one grade in about 20%, or in about 25%, or in about 30%, or in about 35%, or in about 40% or more of the patients receiving EGFRI, four weeks after the end of the treatment with the hydrophobic foam composition or gel.

In one embodiment the pre-emptive treatment with the hydrophobic foam composition or gel provided herein is administered in any daily dosage regime described in this application.

In one or more embodiments, the pre-emptive treatment with the hydrophobic foam composition or gel provided herein is safe and tolerated when the hydrophobic gel or foam composition is administered twice daily for a period of at least five weeks.

In one or more embodiments, the tolerability of the hydrophobic foam composition or gel used in the method is determined by skin irritation and wherein symptoms for assessing skin irritation are selected from a group consisting of pigmentation, erythema, dryness, peeling, and itching.

In one or more embodiments, topical application of the hydrophobic foam composition or gel is safe, and tolerated and has high rates of clinical responses when the hydrophobic gel or foam composition is administered twice daily for at least two weeks.

In one or more embodiments, the method comprises a step of administering, which includes releasing the hydrophobic gel or foam composition from a container and applying it onto the target area by collapsing and/or spreading it on the target area using mild mechanical force thereby resulting in the hydrophobic gel or foam composition collapsing and being absorbed onto the target area. In one or more embodiments, the method further comprises using a sterile applicator or prior to the steps of administering and/or collapsing and/or spreading, the hands of the person spreading are sterilized in order to avoid cross contamination.

In one or more embodiments, topical application by collapsing and/or spreading the hydrophobic gel or foam composition onto a skin or mucosal surface can result in the composition being absorbed within at least 120 seconds.

In one or more embodiments, the pre-emptive treatment with hydrophobic gel or foam composition for use in the methods provided herein results in a decrease rash severity in at least one grade after twelve weeks or less than twelve weeks of treatment, when the composition is administered on average once daily.

In one or more embodiments, the subject is under the age of thirty or forty, or fifty, or sixty or seventy.

In one embodiment the subject is under the age of forty-six and/or is a pregnant or breastfeeding female.

In one or more embodiments, pre-emptive treatment with a hydrophobic gel or foam composition for use in the methods provided herein results in a decrease in grade of rash after six weeks or less than six weeks of treatment, when the composition is administered on average once daily.

In one or more embodiments, the hydrophobic gel or foam composition for use in the method obtains a decrease in the grade of the rash or in the number of lesions or in the area covered by the lesions or in the severity of the lesions after three weeks or less than three weeks of treatment, when the composition is administered on average once daily. In one or more embodiments, the hydrophobic gel or foam composition for use in the method has a shelf life of at least one or at least two years at ambient temperature.

In one or more embodiments, the hydrophobic gel or foam composition for use in the method has a shelf life of at least two years at refrigerator temperature.

In one or more embodiments, there is provided a method for preventing, retarding, arresting, or reversing the progression of a disorder in a mammalian subject in need thereof, the disorder selected from the group consisting of EGFRI associated rash, EGFRI associated rash related symptoms, a tetracycline antibiotic responsive EGFRI associated rash related disorder, a tetracycline antibiotic responsive EGFRI associated skin disorder, an EGFRI associated skin disorder caused by a bacteria, an EGFRI associated tetracycline antibiotic responsive disorder, an EGFRI associated sebaceous gland disorder, bacteria associated with EGFRI associated lesions, and other superficial infections, including skin infections, the method comprising topically applying to the skin of the subject a hydrophobic foam composition or gel comprising a tetracycline antibiotic at least alternate days or once a day or twice a day for at least five weeks, thereby preventing retarding, arresting, or reversing the progression of the disorder in the subject.

In one or more embodiments, there is provided a method for retarding, arresting, or reversing the progression of a disorder wherein the hydrophobic gel or foam composition comprises:

about 60% to about 99% by weight of at least one hydrophobic solvent;
at least one viscosity-modifying agent selected from the group consisting of a fatty alcohol, a fatty acid, and a wax; and
a therapeutically effective amount of a tetracycline antibiotic.

In one or more embodiments, there is provided a method for retarding, arresting, or reversing the progression of a disorder wherein the hydrophobic gel or foam composition comprises:

about 60% to about 99% by weight of at least one hydrophobic solvent or carrier;
at least one viscosity-modifying agent selected from the group consisting of a fatty alcohol, a fatty acid, and a wax;
a therapeutically effective amount of a tetracycline antibiotic; and
an additional active agent.

In one or more embodiments, there is provided a method for retarding, arresting, or reversing the progression of a disorder wherein the tetracycline antibiotic is selected from the group consisting of a tetracycline, an oxytetracycline, a demeclocycline, a doxycycline, doxycycline hyclate, a lymecycline, a meclocycline, a methacycline, a minocycline, minocycline hydrochloride, a rolitetracycline, a chlorotetracycline, a tigecycline and a mixture of any two or more thereof. In one or more embodiments, there is provided a method for preventing, retarding, arresting, or reversing the progression of a disorder wherein the tetracycline antibiotic is doxycycline hyclate or minocycline hydrochloride. In one or more embodiments, there is provided a method for retarding, arresting, or reversing the progression of a disorder wherein the tetracycline antibiotic is doxycycline hyclate or minocycline hydrochloride at a concentration of from about 1% to about 4% by weight.

In one or more embodiments, provided is a topical composition comprising a tetracycline antibiotic, for preventing or treating an EGFR inhibitor induced skin, nail, or mucosal disorder in a subject, wherein said preventing or treating comprises topically administering the composition prior to and/or during systemic administration of the EGFR inhibitor to at least a portion off the skin, nail, or mucosa of the subject. In one or more embodiments, the composition is a composition provided or described herein. In one or more embodiments, the composition is administered according to a method, regime and/or frequency provided or described herein.

In one or more embodiments, provided is a topical composition comprising a tetracycline antibiotic, for reducing the risk of skin, nail, or mucosal side effects associated with systemic EGFR inhibitor treatment in a subject, wherein said reducing comprises topically administering prior to and/or during the systemic EGFR inhibitor administration the composition to at least a portion of the skin, nail, or mucosa of the subject. In one or more embodiments, the composition is a composition provided or described herein. In one or more embodiments, the composition is administered according to a method, regime and/or frequency provided or described herein.

In one or more embodiments, provided is a composition comprising a tetracycline antibiotic selected from the group consisting of cetuximab, panitumumab, zalutumumab, nimotuzumab, matuzumab, erlotinib, gefitinib, lapatinib, canertinib, vandetanib, and mixtures of any two or more thereof, for preventing or treating a drug-induced dermatose comprising a non follicular rash in an subject by topically administering the composition for a period of at least 5 weeks to at least a portion of the skin, nails, or mucosa of the subject prior to and/or during systemic administration of the drug to the patient. In one or more embodiments, the composition is a composition provided or described herein. In one or more embodiments, the composition is administered according to a method, regime and/or frequency provided or described herein.

In one or more embodiments, provided is a topical formulation of a tetracycline antibiotic, for preventing or treating an EGFR inhibitor induced adverse effect on the skin, nails, or mucosal membranes in a patient, which adverse effect is selected from the group consisting of skin rash, skin redness, skin dryness, nail infection, cracking swelling or sores of the lips or corners of the mouth, dermatitis acneiform, itchy skin, stomatitis and paronychia, wherein said preventing or treating comprises administering the topical formulation to at least a portion of the adversely affected area. In one or more embodiments, the composition is a composition provided or described herein. In one or more embodiments, the composition is administered according to a method, regime and/or frequency provided or described herein.

In one or more embodiments, provided is a topical composition comprising a tetracycline antibiotic, for preventing or treating a tyrosine kinase inhibitor induced skin, nail, or mucosal disorder in a subject, wherein said preventing or treating comprises topically administering the composition to at least a portion of the skin, nail, or mucosa of the subject prior to and/or during systemic administration of the EGFR inhibitor. In one or more embodiments, the composition is a composition provided or described herein. In one or more embodiments, the composition is administered according to a method, regime and/or frequency provided or described herein.

In one or more embodiments, provided is a topical composition comprising a tetracycline antibiotic, for reducing the risk of skin, nail, or mucosal side effects associated with systemic tyrosine kinase inhibitor treatment in a subject, wherein said reducing comprises topically administering the composition to at least a portion of the skin, nail, or mucosa of the subject prior to and/or during the systemic tirosine kinase inhibitor administration. In one or more embodiments, the composition is a composition provided or described herein. In one or more embodiments, the composition is administered according to a method, regime and/or frequency provided or described herein.

In one or more embodiments, provided is the use of a tetracycline antibiotic in the manufacture of a topical composition for preventing or treating an EGFR inhibitor induced skin, nail, or mucosal disorder in a subject, wherein said preventing or treating comprises topically administering the composition prior to and/or during systemic administration of the EGFR inhibitor to at least a portion off the skin, nail, or mucosa of the subject. In one or more embodiments, the composition is a composition provided or described herein. In one or more embodiments, the composition is administered according to a method, regime and/or frequency provided or described herein.

In one or more embodiments, provided is the use of a tetracycline antibiotic in the manufacture of a topical composition for reducing the risk of skin, nail, or mucosal side effects associated with systemic EGFR inhibitor treatment in a subject, wherein said reducing comprises topically administering prior to and/or during the systemic EGFR inhibitor administration the composition to at least a portion of the skin, nail, or mucosa of the subject. In one or more embodiments, the composition is a composition provided or described herein. In one or more embodiments, the composition is administered according to a method, regime and/or frequency provided or described herein.

In one or more embodiments, provided is the use of a tetracycline antibiotic selected from the group consisting of cetuximab, panitumumab, zalutumumab, nimotuzumab, matuzumab, erlotinib, gefitinib, lapatinib, canertinib, vandetanib, and mixtures of any two or more thereof, in the manufacture of a composition for preventing or treating a drug-induced dermatose comprising a non follicular rash in an subject by topically administering the composition for a period of at least 5 weeks to at least a portion of the skin, nails, or mucosa of the subject prior to and/or during systemic administration of the drug to the patient. In one or more embodiments, the composition is a composition provided or described herein. In one or more embodiments, the composition is administered according to a method, regime and/or frequency provided or described herein.

In one or more embodiments, provided is the use of a tetracycline antibiotic in the manufacture of a topical formulation for preventing or treating an EGFR inhibitor induced adverse effect on the skin, nails, or mucosal membranes in a patient, which adverse effect is selected from the group consisting of skin rash, skin redness, skin dryness, nail infection, cracking swelling or sores of the lips or corners of the mouth, dermatitis acneiform, itchy skin, stomatitis and paronychia, wherein said preventing or treating comprises administering the topical formulation to at least a portion of the adversely affected area. In one or more embodiments, the composition is a composition provided or described herein. In one or more embodiments, the composition is administered according to a method, regime and/or frequency provided or described herein.

In one or more embodiments, provided is the use of a tetracycline antibiotic in the manufacture of a topical composition for preventing or treating a tyrosine kinase inhibitor induced skin, nail, or mucosal disorder in a subject, wherein said preventing or treating comprises topically administering the composition to at least a portion of the skin, nail, or mucosa of the subject prior to and/or during systemic administration of the EGFR inhibitor. In one or more embodiments, the composition is a composition provided or described herein. In one or more embodiments, the composition is administered according to a method, regime and/or frequency provided or described herein.

In one or more embodiments, provided is the use of a tetracycline antibiotic in the manufacture of a topical composition for reducing the risk of skin, nail, or mucosal side effects associated with systemic tyrosine kinase inhibitor treatment in a subject, wherein said reducing comprises topically administering the composition to at least a portion of the skin, nail, or mucosa of the subject prior to and/or during the systemic tyrosine kinase inhibitor administration. In one or more embodiments, the composition is a composition provided or described herein. In one or more embodiments, the composition is administered according to a method, regime and/or frequency provided or described herein.

In one or more embodiments, a patient may be given radiation therapy in conjunction with EGFRI therapy. In some embodiments, the radiotherapy can start before EGFRI therapy. In some embodiments, radiotherapy can commence after EGFRI therapy. In some embodiments it can be during the same time period. In some embodiments, radiotherapy and EGFRI therapy can overlap for part of the therapy. In some embodiments, radiotherapy can be given on its own. In any of the aforesaid cases, in one or more embodiments, a topical composition provided or described herein may be applied to a skin, nail mucosal or body cavity surface. In some embodiments, the method is a method for preventing or treating an EGFR inhibitor induced skin, nail, or mucosal disorder in a subject, comprising topically administering prior to and/or during systemic administration of the EGFR inhibitor and or radiation therapy a topical composition comprising a tetracycline antibiotic to at least a portion off the skin, nail, or mucosa of the subject. In some embodiments, there is provided a method for reducing the risk of skin, nail, or mucosal side effects associated with systemic EGFR inhibitor treatment and/or radiation therapy in a subject, the method comprising topically administering prior to and/or during the systemic EGFR inhibitor administration a topical composition comprising a tetracycline antibiotic to at least a portion of the skin, nail, or mucosa of the subject. In some embodiments, there is provided a method for preventing or treating a drug-induced and/or radiation-induced dermatose comprising a non follicular rash in an subject, the method comprising topically administering a composition comprising a tetracycline antibiotic, for a period of at least 5 weeks to at least a portion of the skin, nails, or mucosa of the subject prior to and/or during systemic administration of the drug and/or radiation therapy to the patient, wherein the drug is selected from the group consisting of cetuximab, panitumumab, zalutumumab, nimotuzumab, matuzumab, erlotinib, gefitinib, lapatinib, canertinib, vandetanib, and mixtures of any two or more thereof. In some embodiments, a method for preventing or treating an EGFR inhibitor and/or radiation therapy induced adverse effect of the skin, nails, or mucosal membranes in a patient in need thereof, the method comprising administering a topical composition of a tetracycline antibiotic to at least a portion of the adversely affected area; wherein the adverse effect is selected from the group consisting of skin rash; skin redness; skin dryness; nail infection; cracking, swelling, or sores of the lips or corners of the mouth; dermatitis acneiform; itchy skin; stomatitis; and paronychia. In one or more embodiments, there is provided a method for preventing or treating a tyrosine kinase inhibitor and/or radiation therapy induced skin, nail, or mucosal disorder in a subject, comprising topically administering prior to and/or during systemic administration of the EGFR inhibitor and/or radiation therapy a topical composition comprising a tetracycline antibiotic to at least a portion off the skin, nail, or mucosa of the subject. In some embodiments, a method for reducing the risk of skin, nail, or mucosal side effects associated with systemic tyrosine kinase inhibitor treatment and/or radiation therapy in a subject, the method comprising topically administering prior to and/or during the systemic tyrosine kinase inhibitor and/or radiation therapy administration a topical composition comprising a tetracycline antibiotic to at least a portion of the skin, nail, or mucosa of the subject. As used herein, the term “radiation” has the meaning of radiation therapy given to cancer patients rather than natural UV-induced radiation.

In some embodiments there is provided a method for preventing, reducing or treating an radiation therapy induced skin or mucosal disorder in a subject, comprising topically administering prior to and/or during administration of the radiation therapy a topical composition comprising a tetracycline antibiotic to at least a portion off the skin or mucosa of the subject. In some embodiments a topical composition comprising a tetracycline antibiotic is for use in the method. In some embodiments use of a topical composition comprising a tetracycline antibiotic in the manufacture of a medicament having activity against radiation therapy induced dermatitis is provided.

In some embodiments there is provided a method for reducing the risk of skin or mucosal side effects associated with radiation therapy in a subject, the method comprising topically administering prior to and/or during the radiation therapy administration a topical composition comprising a tetracycline antibiotic to at least a portion of the skin or mucosa of the subject. In some embodiments a topical composition comprising a tetracycline antibiotic is for use in the method. In some embodiments use of a topical composition comprising a tetracycline antibiotic in the manufacture of a medicament having activity against radiation therapy induced dermatitis is provided.

A method for preventing or treating a radiation therapy induced adverse effect of the skin or mucosal membranes in a patient in need thereof, the method comprising administering a topical composition of a tetracycline antibiotic to at least a portion of the adversely affected area, wherein the adverse effect is selected from the group consisting of grade 1 or grade 2 or grade three or grade four dermatitis, or is selected from the group consisting of faint erythema, redness, desquamation, itchy skin, peeling skin, moist desquamation, open wound, skin necrosis, death of skin cells and ulceration. In some embodiments a topical composition comprising a tetracycline antibiotic is for use in the method. In some embodiments use of a topical composition comprising a tetracycline antibiotic in the manufacture of a medicament having activity against radiation therapy induced dermatitis is provided.

Where embodiments of the present invention are discussed herein in terms of a method of treatment involving the administration of a formulation or composition, it will be understood that the invention also provides that formulation, composition or active ingredient(s) thereof for use in that method, as well as the use of the formulation, composition or active ingredient(s) thereof in the manufacture of a medicament for use in that method

### DESCRIPTION OF DRAWINGS

FIG. 1 represents a visual scale of rash severity, indicating mild, moderate, and severe grades of rash.

FIG. 2 shows the mean (SD) minocycline plasma concentration from Day 1 to Day 16 for subjects who received FMX-101 (minocycline HCl foam, 4%).

FIG. 3 is a graph showing the probability of remaining free of severe rash from the start of EGFRI treatment to the time point of developing grade 3 rash (GSS), by treatment side (ITT analysis).

FIG. 4 shows that FDX104 is useful in preventing severe acneiform rash and prevented the development of or reduced the severity of such a rash in more than forty percent of the cases.

### DETAILED DESCRIPTION

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

All % values are provided on a weight (w/w) basis.

Various carriers and compositions or formulations are described herein. They are often described for use in a method. A reference to or example of a carrier, composition or formulation for use in one method does not in any way limit the carrier, composition or formulation for use just in that method, but it can be for use in any other method or embodiment described herein. The carriers, compositions or formulations described herein are in one or more embodiments provided as carriers, compositions or formulations and are in one or more embodiments provided as a product even where they are described only in relation to their use in a method.

As used herein, the term “about” has its usual meaning in the context of pharmaceutical and cosmetic formulations to allow for reasonable variations in amounts that can achieve the same effect. By the term “about” herein it is meant as indicated above and also that a figure or range of figures can vary in an embodiment plus or minus up to 30%. For example, if an amount of “about 1” is provided, then the amount can be up to 1.3 or from 0.70. In other embodiments, it can reflect a variation of plus or minus 20%, in which case “about 2” can reflect a variation of 1.6 to 2.4. In still further embodiments, it can describe a variation of plus or minus 10%, in which case “about 1” can reflect a variation of 0.9 to 1.1. In still further embodiments, it can describe a variation of plus or minus 5%, in which case “about 5” can reflect a variation of 4.75 to 5.25. In cases where “about X” will lead to a figure of above 100%, the term in one or more embodiments can be read as reflecting up to 100% by weight less the total of the minimum amount of the other ingredients. Likewise, it will be appreciated by one skilled in the art to the extent X is reduced from that upper level the amounts of the other ingredients are increased appropriately. As will be appreciated by one of skill in the art, there is some reasonable flexibility in formulating compositions such that where one or more ingredients are varied, successful formulations can still be made even if an amount falls slightly outside the range. Therefore, to allow for this possibility, amounts are qualified by about. In one or more other embodiments, the figures may be read without the term “about.”

In one or more embodiments, the terms “composition(s)” and “formulation(s)” can be interchangeable depending on the context in which they are used as would be appreciated by a person skilled in the art.

The term “room temperature” as used herein, means 20° C. to 22° C. In an embodiment it is 20° C. In an embodiment it is 21° C. In an embodiment it is 22° C.

The term “thixotropic,” as used herein, means that the formulation shows a decrease in viscosity upon application of shear force. The structure of the formulation breaks down, leading to a reduction in viscosity. When the formulation is standing without shear force, this decrease in viscosity is recovered over time.

As used herein, the term “gel” means a jelly-like material that can have properties ranging from soft and fluid to hard and tough. Gels can be in liquid, semi-liquid, semi-solid or solid state. Solid gels are defined as a substantially diluted crosslinked system, which exhibits no flow when in the steady-state. By weight, gels are mostly liquid, yet they behave like semi-solids due to a three-dimensional crosslinked network of a solidifying, gelling or thickening agent within the liquid. It is the crosslinks within the fluid that give a gel its structure (hardness) and contribute to stickiness (tack). Depending on the amounts of gelling agent in a formulation, the gel may be semi-solid with some limited flowability, such that when the semi-solid gel is placed in a tube and is inclined horizontally from a vertical position it will slowly flow from the vertical towards the horizontal or it may be a liquid gel where the amount of gelling agent or gelling effect is lower, such that the gel structure or connections are weaker or loose so that when placed in a tube and tilted from a vertical position to a horizontal position, the gel readily flows and adapts to the horizontal position. The rheological properties of gels at different surface temperatures can influence the release and bioabsorption of drugs therefrom.

In one or more embodiments, the gel is stable and it retains its viscosity upon dispensing from a container, such as a tube, yet, it liquefies and spreads easily upon application of shear force, which can be mild, such as a simple rub. Further, while the gel is oily, it absorbs into the site of application, such as the skin or mucosa membrane, and after minutes the surface does not appear and/or feel significantly oily or greasy.

The term “liquid gel” refers inter alia to a formulation after propellant is added (which prior to adding the propellant is a gel) or where the gel is loose or fluid or such that when subjected to gravity will pour or become liquid.

The terms “waterless” or “water-free” as used herein, mean that the composition contains no free or unassociated or absorbed water. The terms “substantially water-free” or “substantially waterless” refer to carriers that contain at most incidental or trace amounts of water. In one or more embodiments, “low water” means the composition contains about or less than 1% by weight; about or less than 0.9% by weight; about or less than 0.8% by weight; about or less than 0.7% by weight; or about or less than 0.6% by weight. In one or more embodiments, “substantially waterless” or “substantially water free” means the composition contains about or less than 0.5% by weight; about or less than 0.4% by weight; about or less than 0.3% by weight; about or less than 0.2% by weight; about or less than 0.1% by weight; about or less than 0.05% by weight; or about or less than 0.01% by weight water. In one or more embodiments, the composition is “essentially water-free,” indicating about or less than 0.05% water by weight.

By the term “single phase” it is meant that after addition of propellant to the composition or carrier, the liquid components of the foamable composition or carrier are fully miscible, and the solid components, if any, are either dissolved or homogeneously suspended in the composition so that only one phase is visible.

By the term “substantially a single phase” it is meant that the composition or carrier, after addition of propellant, is primarily or essentially a single phase as explained above, but can also have present a small amount of material which is capable of forming a separate phase amounting to less than about 5% by weight of the composition or carrier after the addition of propellant, or less than about 3% by weight, and/or less than about 1% by weight of the composition.

The term “unstable” as used herein, means a compound, e.g., an active agent, which is oxidized and/or degraded within less than a day, and in some cases, in less than an hour, upon exposure to air, light, skin, or water or a pharmaceutical excipient under ambient conditions.

It should be noted that the terms “surfactant,” “surface active agent,” and “emulsifier” in the context used herein, refer to stand alone compounds used to reduce surface tension between two substances or phases, and which are also capable of stabilizing an emulsion of water and oil. Reduction of surface tension can be significant in foam technology in relation to the ability to create small stable bubbles. “Surfactant” and “emulsifier,” as used herein, do not include compounds which do not function effectively as standalone compounds for reducing surface tension between two substances or phases and which are not capable of stabilizing an emulsion of water and oil. For example, a surfactant or emulsifier as provided herein does not include fatty acids, does not include fatty alcohols, and does not include propoxylated lanolin oil derivatives. In the context of the present invention, fatty acids and fatty alcohols are defined as foam adjuvants. Similarly, propoxylated lanolin oil derivatives in the context herein are defined as emollients.

“Standard surfactant,” “customary surfactant” or “stand alone surfactant” refer to customary non-ionic, anionic, cationic, zwitterionic, amphoteric and amphiphilic surfactants. Many standard surfactants are derivatives of fatty alcohols or fatty acids, such as ethers or esters formed from such fatty alcohols or fatty acids with hydrophilic moieties, such as polyethylene glycol (PEG). However, a native (non-derivatized) fatty alcohol or fatty acid, as well as waxes are not regarded as a standard surfactant.

The term “co-surfactant” as used herein means a molecule which on its own is not able to form and stabilize satisfactorily an oil-in-water emulsion, but when used in combination with a surfactant as defined herein, the co-surfactant has properties which can allow it to help a surfactant create an emulsion and can boost the stabilizing power or effect of the surfactant. Examples of co-surfactants include fatty alcohols, such as cetyl alcohol, or fatty acids, such as stearic acid. Cetyl alcohol is a waxy hydrophobic substance that can be emulsified with water using a surfactant. Some substances can have more than one function and for example, fatty alcohols can in some formulations act as a co-solvent. In certain circumstances, a co-surfactant can itself be converted into a surfactant or soap by, for example, adding a base, such as, triethanolamine to a fatty acid like stearic acid.

The term “viscosity-modifying agent” in the context of the present disclosure is an agent which, when added to a hydrophobic oil, facilitates the creation of a hydrophobic breakable vehicle in the form of a breakable gel or breakable foam. In one or more embodiments, the viscosity-modifying agent is a “foamer complex” comprising a fatty alcohol, a fatty acid and/or a wax. In one or more alternative embodiments the foamer complex is a fatty alcohol and a wax or a fatty acid and a wax. In some embodiments it is a wax. In some embodiments a fatty alcohol, and or a fatty acid and or a wax is an adjuvant. In the context of the present disclosure fatty alcohols, fatty acids and waxes that are compatible with tetracycline antibiotics, and in particular with a minocycline or a doxycycline, are compatible adjuvants.

The term “breakable” refers to a property of a gel or foam wherein the gel or foam is stable upon dispensing from a container, yet breaks and spreads easily upon application of shear or mechanical force, which can be mild, such as a simple rub.

It should be noted that the term “polyol” as used herein is an organic substance that contains at least two hydroxy groups in its molecular structure.

The term “water activity” as used herein represents the hygroscopic nature of a substance, or the tendency of a substance to absorb water from its surroundings. Microorganisms require water to grow and reproduce, and such water requirements are best defined in terms of water activity of the substrate. The water activity of a solution is expressed as Aw=P/Po, where P is the water vapor pressure of the solution and Po is the vapor pressure of pure water at the same temperature. Every microorganism has a limiting Aw, below which it will not grow; e.g., for Streptococci, Klebsiella spp, Escherichia coli, Clostridium perfringens, and Pseudomonas spp, the Aw value is 0.95. Staphylococcus aureus is most resistant and can proliferate with an Aw as low as 0.86, and fungi can survive at an Aw of at least 0.7.

In one embodiment, no preservative is needed in the formulations provided herein because the formulation is a waterless hydrophobic solvent or oil-based formulation having an Aw (water activity) value of less than 0.9, or less than about 0.8, or less than about 0.7, or less than about 0.6, and/or less than about 0.5, which is below the level of microbial proliferation.

The identification of a “solvent,” as used herein, is not intended to characterize the solubilization capabilities of the solvent for any specific active agent or any other component of the foamable composition. Rather, such information is provided to aid in the identification of materials suitable for use as a component of the foamable composition described herein.

As used herein, the term “preventing” refers to avoiding the onset of a disorder or condition from occurring in a subject which has not yet been diagnosed as having the disorder or condition, but who may be susceptible to it.

As used herein, the term “treatment” refers to inhibiting the disorder or condition, i.e., arresting its development; relieving the disorder or condition, i.e., causing regression of the disorder or condition or reversing the progression of the disorder or condition; or relieving or reducing one or more symptoms of the disorder or condition.

It should be noted that the term “a method of preventing, treating a disease or a disorder” as provided throughout the specification is interchangeable with the term “use of the composition as a medicament for preventing or treating a disease.” It should be noted that the term “disease” is used interchangeably with the term “disorder.”

It should be noted that the term “substantially free of” an ingredient as provided throughout the specification is intended to mean that the composition comprises less than about 0.5% by weight (e.g., less than about 0.4% by weight, less than about 0.3% by weight, less than about 0.2% by weight, less than about 0.1% by weight, less than about 0.05% by weight, less than about 0.01% by weight, less than about 0.001% by weight, or 0% by weight) of an ingredient unless specifically indicated otherwise.

As used herein, the term “essentially free of” an ingredient as provided throughout the specification is intended to mean that the composition comprises less than about 0.05% by weight, less than about 0.01% by weight, less than about 0.001% by weight, or 0% by weight, or insignificant or negligible amounts of the ingredient, unless specifically indicated otherwise.

As used herein, the term “free of” an ingredient as provided throughout the specification is intended to mean that the composition does not comprise any amount of the ingredient, unless specifically indicated otherwise.

The terms “surfactant-free” or “emulsifier-free” or “non-surfactant” refer to compositions which comprise no or negligible levels of surfactants, emulsifiers, or surface active agents. Where a formulation includes insignificant or de minimis amounts of surfactants, emulsifiers, or surface active agents it is considered to be essentially surfactant-free. In one or more embodiments, “essentially free” indicates less than about 0.05% by weight, less than about 0.01% by weight, less than about 0.001% by weight, or 0% by weight.

The term “substantially surfactant-free” relates to a composition wherein the ratio between the viscosity-modifying agent and the surfactant is between 10:1 or 5:1; or between 20:1 and 10:1 or between 100:1 and 20:1. In additional embodiments, the term relates to a composition that contains a total of about or less than 0.5% by weight; about or less than 0.4% by weight; or about or less than 0.3% by weight of a surfactant selected from the group consisting of customary non-ionic, anionic, cationic, zwitterionic, amphoteric and ampholytic surfactants. In some embodiments, the composition comprises about or less than 0.2% by weight of a standard or customary surfactant; about or less than 0.15% by weight; about or less than 0.1% by weight; about or less than 0.05% by weight; or about or less than 0.01% by weight.

By “de minimis” it is meant to be so minor as to merit disregard.

The terms “hydrophobic gel composition” or “hydrophobic foam composition” or “hydrophobic composition” are intended to mean that the composition has a low solubility in water. In one embodiment, 100 to 1000 parts of water are needed to dissolve or render miscible 1 part of the composition. In another embodiment, 1000 to 10,000 parts of water are needed to dissolve or render miscible 1 part of the composition. In yet another embodiment, more than 10,000 parts of water are needed to dissolve or render miscible 1 part of the composition.

By “regular basis” it is meant a repeated or repeatable interval of time which can be by way of illustration, a part of a day, daily, twice daily, alternative daily, alternate daily, twice weekly, weekly, fortnightly, monthly or some other repeated or repeatable interval for an appropriate period of time wherein a dose is to be applied. The repeated applications can be determined according to the needs of the subject and the disease or disorder. In some circumstances as little as three repeat doses can be required. In other cases, between 3 and 14, in other cases between 14 and 28, in other cases between 28 and 50, in other cases between 50 and 75, in other cases between 75 and 100, and in other cases, such as where prolonged treatment or a long period of maintenance dosing is needed, as many as one, two, or three hundred repeat doses can be needed.

The term “hazard ratio” as used herein refers to the ratio of hazard rates corresponding to the conditions described by two levels of an explanatory variable. For example, in the studies provided herein, if the untreated population is twice as likely to exhibit a more severe rash as compared to the treated population, the hazard ratio would be 2, indicating a higher hazard of developing a more severe rash.

In one or more embodiments, there is provided herein a method for treating a disorder of the skin or a mucosal surface, or a disorder of a sebaceous gland. In one or more embodiments, the disorder is a consequence or unwanted side effect of exposure to or treatment with pharmaceutical active agents. In one or more embodiments, the disorder is a consequence or unwanted side effect of exposure to or treatment with pharmaceutical active agents in combination with exposure to radiation. In one or more embodiments, the disorder is a consequence or unwanted side effect of treatment with or exposure to an EGFRI. In one or more other embodiments, the disorder is a consequence or unwanted side effect of treatment with or exposure to an EGFRI either in combination with exposure to radiation or in combination with treatment with other pharmaceutical active agents either simultaneously, consecutively or overlapping.

In one or more embodiments, there is provided herein, a method for treating a disorder including one or more of a EGFRI associated rash, EGFRI associated rash related symptoms, a tetracycline antibiotic responsive EGFRI associated rash related disorder, a EGFRI associated tetracycline antibiotic responsive skin disorder, an EGFRI associated skin disorder caused by a bacteria, an EGFRI associated tetracycline antibiotic responsive disorder, an EGFRI associated sebaceous gland disorder, a bacterial infection resulting from EGFRI treatment and other superficial infection, including skin infections. In one or more embodiments, the disorder does not involve inflammation. In another embodiment the disorder does involve inflammation.

In one or more embodiments, the method includes administering topically to a surface affected by a disorder a therapeutic hydrophobic composition prior to and for the duration of EGFRI treatment, the composition consisting of a carrier comprising about 60% to about 95% by weight of at least one hydrophobic solvent; at least one viscosity-modifying agent selected from the group consisting of a fatty alcohol, a fatty acid and a wax; and a tetracycline antibiotic.

In one or more embodiments, the method includes administering topically to a surface affected by a disorder a therapeutic hydrophobic composition prior to and for the duration of EGFRI treatment, the composition consisting of a carrier comprising about 60% to about 95% by weight of at least one hydrophobic solvent; at least one viscosity-modifying agent selected from the group consisting of a fatty alcohol, a fatty acid and a wax; a tetracycline antibiotic; and an additional active agent.

In one or more embodiments, the treatment is consecutive or in parallel with the EGFRI treatment. In one or more embodiments, the treatment is commenced in response to or upon the appearance of the unwanted disorder or side effect. In one or more embodiments, the treatment is continued for a period of time after cessation of the EGFRI treatment, for example, about a week, or about two weeks, or about three weeks, or about four weeks, or about five weeks, or about six weeks, or about seven weeks, or about eight weeks, or about nine weeks, or about ten weeks, or about eleven weeks, or about twelve weeks after cessation of the EGFRI treatment.

In one or more embodiments, there is provided a method of treating or alleviating an EGFRI associated rash in a subject having exposure to an EGFRI either in combination with exposure to radiation or in combination with treatment with other pharmaceutical active agents or both. In one or more embodiments, there is provided a method of treating or alleviating EGFRI associated rash related symptoms either in combination with exposure to radiation or in combination with treatment with other pharmaceutical active agents or both. In one or more embodiments, there is provided a method of treating or alleviating a tetracycline antibiotic responsive EGFRI associated rash related disorder either in combination with exposure to radiation or in combination with treatment with other pharmaceutical active agents or both. In one or more embodiments, there is provided a method of treating or alleviating a superficial skin or mucosal disorder that is a by-product of a therapeutic treatment or treatment regime applied to a subject including EGFRI therapy.

In one or more embodiments, the method includes administering topically to a surface affected by a disorder a therapeutic hydrophobic composition prior to and for the duration of EGFRI treatment alone or in combination with radiation or another active pharmaceutical agent, consisting of a carrier comprising about 60% to about 95% by weight of at least one hydrophobic solvent; at least one viscosity-modifying agent selected from the group consisting of a fatty alcohol, a fatty acid and a wax; and a tetracycline antibiotic.

In one or more embodiments, the method includes administering topically to a surface affected by a disorder a therapeutic hydrophobic composition prior to and for the duration of EGFRI treatment alone or in combination with radiation or another active pharmaceutical agent, consisting of a carrier comprising about 60% to about 95% by weight of at least one hydrophobic solvent; at least one viscosity-modifying agent selected from the group consisting of a fatty alcohol, a fatty acid and a wax; a tetracycline antibiotic; and an additional active agent.

In one or more embodiments, the treatment is consecutive or in parallel with the other pharmaceutical agent or radiation treatment. In one or more embodiments, the treatment is commenced in response to or upon the appearance of the unwanted disorder or side effect. In one or more embodiments, the treatment is continued for a period of time after cessation of the other pharmaceutical agent or radiation treatment, for example, about a week, or about two, weeks, or about three weeks, or about four weeks, or about five weeks, or about six weeks, or about seven weeks, or about eight weeks, or about nine weeks, or about ten weeks, or about eleven weeks, or about twelve weeks after cessation.

According to one or more embodiments provided herein, the tetracycline is a minocycline or doxycycline, which are semi-synthetic tetracycline antibiotics. According to one or more embodiments, the tetracycline is minocycline. According to one or more embodiments, the tetracycline is a doxycycline or doxycycline hyclate (hereinafter “doxycycline” or DOX”). The tetracycline drug is usually bacteriostatic in action. It can, amongst other options, exert its antimicrobial activity by inhibiting protein synthesis. It can also have an antiviral effect. According to one or more embodiments the minocycline is minocycline hydrochloride (minocycline HCl; (hereinafter “MCH”)). MCH is a yellow crystalline powder that is sparingly soluble in water, slightly soluble in alcohol and practically insoluble in chloroform and in ether.

Tetracycline antibiotics like minocycline and doxycycline are sensitive to breakdown or degradation. For example, minocycline is known to be highly sensitive to air and light and undergoes rapid degradation. Therefore, storage of foamable formulations in airtight sealed containers under pressure with propellant can contribute to preserving stability subject to selection of compatible canisters and accessories. Likewise, production and/or filling under vacuum in an oxygen free environment can help.

The ingredients of the carrier were selected for their compatibility with tetracycline antibiotics as described. It was not sufficient to identify single ingredients that were compatible but formulations had to be found in which the ingredients in combination were also compatible.

In one or more embodiments, a hydrophobic foamable composition (e.g., foam or gel) provided herein comprises:
a) about 60% to about 99% by weight of at least one hydrophobic solvent or carrier;
b) about 1% to about 22% by weight of at least one viscosity modifying agent; and
c) about 0.1% to about 18% by weight of a tetracycline antibiotic (e.g., minocycline HCl or doxycycline hyclate).

In one or more embodiments, a hydrophobic foamable composition (e.g., foam or gel) provided herein comprises:
a) about 60% to about 99% by weight of at least one hydrophobic solvent or carrier;
b) about 1% to about 22% by weight of at least one viscosity modifying agent;
c) about 0.1% to about 18% by weight of a tetracycline antibiotic (e.g., minocycline HCl or doxycycline hyclate); and
d) an additional active agent.

In one or more embodiments, a hydrophobic foamable composition or gel provided herein comprises:
a) about 70% to about 90% by weight of at least one hydrophobic solvent or carrier;
b) about 10 to about 22% by weight of at least one viscosity modifying agent; and
c) about 0.5% to about 8% by weight of a tetracycline antibiotic (e.g., minocycline HCl or doxycycline hyclate).

In one or more embodiments, a hydrophobic foamable composition or gel provided herein comprises:
a) about 70% to about 90% by weight of at least one hydrophobic solvent or carrier;
b) about 10 to about 22% by weight of at least one viscosity modifying agent;
c) about 0.5% to about 8% by weight of a tetracycline antibiotic (e.g., minocycline HCl or doxycycline hyclate); and
d) an additional active agent.

In one or more embodiments, a hydrophobic foamable composition or gel provided herein comprises:
a) about 75% to about 90% by weight of at least one hydrophobic solvent or carrier;
b) about 10 to about 22% by weight of at least one viscosity modifying agent; and
c) about 0.5% to about 2% by weight of a tetracycline antibiotic (e.g., minocycline HCl or doxycycline hyclate).

In one or more embodiments, a hydrophobic foamable composition or gel provided herein comprises:
a) about 75% to about 90% by weight of at least one hydrophobic solvent or carrier;
b) about 10 to about 22% by weight of at least one viscosity modifying agent;
c) about 0.5% to about 2% by weight of a tetracycline antibiotic (e.g., minocycline HCl or doxycycline hyclate); and
d) an additional active agent.

In one or more embodiments, a hydrophobic foamable composition or gel provided herein comprises:
a) about 72% to about 88% by weight of at least one hydrophobic solvent or carrier,
b) about 10 to about 22% by weight of at least one viscosity modifying agent; and
c) about 2% to about 6% by weight of a tetracycline antibiotic (e.g., minocycline HCl or doxycycline hyclate).

In one or more embodiments, a hydrophobic foamable composition or gel provided herein comprises:
a) about 72% to about 88% by weight of at least one hydrophobic solvent or carrier;
b) about 10 to about 22% by weight of at least one viscosity modifying agent;
c) about 2% to about 6% by weight of a tetracycline antibiotic (e.g., minocycline HCl or doxycycline hyclate); and
d) an additional active agent.

According to one or more embodiments, there are provided substantially surfactant-free oleaginous formulations comprising a tetracycline, such as a minocycline, for use in treatment of EGFRI associated rash, EGFRI associated rash related symptoms, a tetracycline antibiotic responsive EGFRI associated rash related disorder, a EGFRI associated tetracycline antibiotic responsive skin disorder, EGFRI associated skin disorder caused by a bacteria, an EGFRI associated tetracycline antibiotic responsive disorder, an EGFRI associated sebaceous gland disorder, EGFRI associated rash resulting in bacteria associated disorders and other superficial infections, including skin infections. In one or more embodiments, the tetracycline acts to reduce oxidative stress and/or inflammation in skin pathologies.

Thus, in one or more embodiments, there is provided a topical composition comprising a tetracycline antibiotic to counteract or ameliorate rash-like side effects, i.e., adverse effects (AEs), of EGFR inhibitors. In one or more embodiments, there is provided a topical composition comprising a tetracycline antibiotic to counteract or ameliorate burning-like side effects, i.e., adverse effects (AEs), of EGFR inhibitors. In one or more embodiments, there is provided a topical composition comprising a tetracycline antibiotic to counteract or ameliorate pain-like side effects, i.e., adverse effects (AEs), of EGFR inhibitors. In one or more embodiments, there is provided a topical composition comprising a tetracycline antibiotic to counteract or ameliorate irritation-like side effects, i.e., adverse effects (AEs), of EGFR inhibitors. In one or more embodiments, there is provided a topical composition comprising a tetracycline antibiotic to counteract or ameliorate itching-like side effects, i.e., adverse effects (AEs), of EGFR inhibitors.

The CTCAE (v 1-4) categorize a broad collection of AEs, but many AEs are not associated with the clinicopathologic events experienced by patients receiving EGFRIs (i.e., CTCAE not specific enough for evaluating these events) and therefore the following methods can be used to assess efficacy.

In one embodiment, the efficacy evaluations are done for the Intent to Treat (ITT) and the Per Protocol (PP) populations. All the efficacy endpoints compared results of treatment side to vehicle side. The efficacy first endpoint is the mean maximal rash grade (exploratory endpoint no. 1) which is a continuous variable. Another efficacy endpoint is the number of incidences with maximal rash grade (exploratory endpoint no. 2), maximal skin rash grade 1 (exploratory endpoint no. 3), maximal skin rash grade 2 (exploratory endpoint no. 4), maximal skin rash grade 3 (exploratory endpoint no. 5) and maximal skin rash grade 2-3 (exploratory endpoint no. 6). The numbers of incidences of the last exploratory endpoints no. 2 to 6 are categorical variables. The distributions for categorical variables are compared and analyzed by the Chi square test (a parametric test), or by Fisher-Irwin exact test (a non-parametric test). Another exploratory endpoint (exploratory endpoint no. 7) is the mean maximal rash grade based on skin photo-type classification, which is a continuous variable. For the efficacies, continuous variable (exploratory endpoints no. 1 and 7) ranges, medians, means and standard deviations are calculated. Test for normality are done by Shapiro-Wilk normality test. The results between pairs of continuous variable are analyzed by T-test for paired (a parametric test) or by Wilcoxon test for paired (a non-parametric test).

The subject compliance, calculated once for the treatment side and once for the vehicle side, can be calculated as the percentage of sum of days that the subject administered the study drug to the sum of days he administrated and did not administrated the study drug. Weighing of consumption study drug calculated once for the treatment side and once for the vehicle side, is calculated as the average study drug per day (the sum of the differences of containers' weighing before and after use divides by the total number of uses days).

The following exploratory analyses can be performed and described.
Summary statistics (median, IQR) of rash score by time.
Spaghetti plots of rash score by time.
Spaghetti plots of difference in rash scores by time.
Summary of time to first moderate or severe score by treatment group.
Summary of time to first severe score by treatment group.
Time difference in time to first moderate or severe score by treatment group.
Time difference in time to first severe score by treatment group.
Comparison of the subset of patients with at least a severe score by group, e.g., median effect size in patients with a maximum score of 3.
Matrix of maximum grade on placebo side to maximum grade on treatment group.
The time (week) when the maximum rash score is first and last recorded.
The time to maximum rash score vs. maximum rash score.
The time to maximum rash score on the placebo side vs. the time to maximum rash score on the treatment side.
Compare to time maximum rash score by patients with +3, +2, +1, 0, −1, and −2, −3 score differences. Repeat this analysis within +1 group by 3v2 and 2v1 groups and similar for 0 and −1 groups.

The term clinical response to treatment, (clinical success or clinical failure) in the context of EGFRI associated rash treatment is derived from an efficacy evaluation.

The term “efficacy” can be assessed by the following methods: (a) MESTT scale; (b) the visual scale of rash severity—Scope photographs method: and (c) a sub-analysis of phototype classification is performed by Fitzpatrick photo-type classification.

(a) MESTT (Multinational Association of Supportive Care in Cancer (MASCC) Skin Toxicity Tool). The eruption rate and grade is assessed according to the MESTT scale, defined by the number of papules or pustules, area of erythema or edema size, pain or pruritus, and effect on emotion or function, which will be evaluated by investigator at each visit.

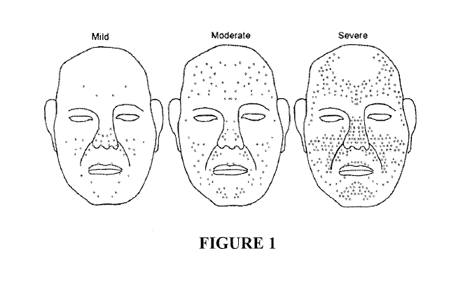

(b) Scope photographs method. The eruption rate and grade is assessed according to: The visual scale of rash severity—Scope A is defined below. The Scope scale is used by a blinded trained clinician in the field, reviewing the digital photographs of the subjects according to the following classification: none, mild, moderate, and severe. FIG. 1 illustrates examples of classifications according to the Scope scale, which include “mild,” “moderate,” and “severe” classifications.

(c) Fitzpatrick photo-type classification (see table below)—is performed at the screening visit and serve for sub-analysis of the efficacy based on skin type.

| | | |
|---|---|---|
| Type I | (scores 0-6) | Pale white; blond or red hair; blue eyes; |
| | | freckles; Always burns, never tans |
| Type II | (scores 7-13) | White; fair; blond or red hair; blue, |
| | | green or hazel eyes Usually burns, tans |
| | | minimally |
| Type III | (scores 14-20) | Cream white; fair with any hair or eye |
| | | color; quite common Sometimes mild |
| | | burn, tans uniformly |
| Type IV | (scores 21-27) | Moderate brown; typical Mediterranean |
| | | skin tone. Rarely burns, always tans |
| | | well |
| Type V | (scores 28-34) | Dark brown; Middle Eastern skin types. |
| | | Very rarely burns, tans very easily |
| Type VI | (scores 35+) | Deeply pigmented dark brown to black. |
| | | Never burns, tans very easily |

In one embodiment, the topical administration of the composition provided herein results in prevention or protection from or reduction of a rash outbreak or EGFR-inhibitor-related dermatose by about 10%, as evaluated using one of the parameters selected from the group consisting of EGFRI-associated cutaneous toxicity grade, CTCAE v3.0 grade for rash, Erythema score, Lesion counts, Pain VAS marked by the subject, Pruritus VAS marked by the subject Photograph of face, Skindex 16, percentage of face surface area involvement, and combinations of any two or more thereof.

The term “safe” in the context herein means having no or essentially no systemic adverse events or serious adverse events related to the study drug. Safety assessments consist of evaluating skin tolerability, adverse events, serious adverse events and vital signs. In those cases of an existing difference in rash severity (using the MESTT scale) between the two facial sides, whereby the more severe grade was observed on the FDX104 treated side laboratory examinations, performance status and documentation of all concomitant medications and/or therapies are included in the safety evaluation.

In one embodiment, safety analysis are done for the safety population. AEs are coded by the CTCAE (version 4.0), SOC (System Organ Class), preferred term and grade. Incidences of AEs are presented by serious adverse events, severity (grade), relationship to study drug, action taken and outcome of event. For the summary by severity, subjects who have multiple occurrences of the same AE are classified according to the worst reported severity of the AE. For the summary by relationship to study drug, subjects who have multiple occurrences of the same AE are classified according to the strongest reported relationship to study medication. The AE variables are categorical variables. For AE, categorical variables numbers and percentages are calculated. Distributions for categorical variables are compared and analyzed by the Chi square test (a parametric test), or by Fisher-Irwin exact test (a non-parametric test).

All the vital signs (SBP, DBP, HR, temperature and respiratory rate) are continuous variables. For the continuous variable ranges, means and standard deviations are calculated. The results between pairs of continuous variable are analyzed by T-test for paired.

The terms “tolerable” or “enhanced tolerability” in the context herein means the degree to which overt adverse effects of a drug can be tolerated by a patient. In one embodiment it is measured by the rate of “dropouts,” or patients that forfeit participation in a study due to extreme adverse effects. In one or more embodiment it is evaluated by the following parameters: vital signs the incidence and severity of AEs (Adverse Events). In one embodiment it is measured by skin irritation events.

The term “adverse events” describes any observed problems in people taking the medicine. The common term for such problems is “side effects,” and is used by patients and physicians.

By “essentially no” in the context of tolerability includes insignificant or de minimis occurrences of dropouts or patients that forfeit participation in a study due to extreme adverse effects.

By “essentially no” in the context of safety includes insignificant or de minimis occurrences of systemic or dermal adverse events or events not connected with the application of topical tetracyclines.

The clinical response can be determined and assessed at each study visit by a clinician using inter alia (a) MESTT scale; (b) the visual scale of rash severity—Scope photographs method; and (c) a sub-analysis of phototype classification, performed by Fitzpatrick photo-type classification.

The term “clinical failure” is defined as insufficient improvement or deterioration (i.e., an increase or no change in rash severity, rash grade or the number of lesions, increase in the number of papules or pustules, area of erythema or edema size, pain or pruritus, effect on emotion or function, % of patients in which no change in the grade of the rash between both side of the face is observed or increase in rash grade is observed in the side receiving doxycycline foam, the time to develop the rash was less than a week).

By “on average” with reference to dosage regimes it is intended to reflect and/or take into account human nature and that a subject may forget to apply a dose or not strictly adhere to the regime, such that even if a subject forgets a dose or does not strictly adhere to the regime it will still be considered as if the regime has been applied. For example, if a subject misses an occasional dose but does not make it up or alternatively, if a subject missed a dose and applies a compensatory dose on a different day, it is still counted as having complied with the dosage regime.

An “EGFRI associated rash related disorder” is any disorder which can occur in parallel with EGFRI associated rash or be a contributing factor to the outbreak of EGFRI associated rash or can resemble EGFRI associated rash.

In EGFRI associated rash, symptoms include papulopustular rash, papules or pustules, erythema, edema, pain, pruritus, effect on emotion or function, sleeping disorders, xerosis, paronychia, and changes in hair growth, ocular side effects such as dry eye, inflammation of the lid margin (blepharitis), dysfunction of the sebaceous glands of the eyelid (meibomitis), long eyelashes (trichomegaly), corneal erosion, and inversion or eversion of the eyelid margin (entropion or ectropion), gastrointestinal side effects (e.g., diarrhea and headache).

In one or more embodiments, topical tetracycline treatments can be given with or followed by application of a steroid or a hyaluronic acid or a collagen or a silicone or mixtures of any two or more thereof, for example to ameliorate or reduce scarring or skin damage effects.

It should be noted that hydrophobic compositions disclosed herein can be applied to the target site as a gel or a semi-solid gel or foam. In certain other embodiments, it can be applied as a liquid gel or as a collapsed foam. In one or more embodiments, the composition is thixotropic. In one or more embodiments, the gel formulation subjected to constant shear rate shows a reduction in viscosity with time. In one or more further embodiments, after the material is allowed to rest for a period of time, the viscosity increases again. In one or more embodiments, there is provided prior to adding propellant a solid or semi-solid composition or gel. In one or more embodiments, the composition or gel is a liquid. In one or more embodiments, the propellant is miscible with and dilutes the composition.

Upon packaging of the foamable composition in an aerosol container and adding a propellant, a shakable and homogenous foamable composition is prepared, which upon dispensing forms a breakable foam with good to excellent quality. The resulting foam is essentially pharmaceutically equivalent to the respective gel (prior to adding the propellant), since immediately upon dispensing of the foam the propellant evaporates and the composition upon collapsing is similar or identical to that of the gel. This is an important pragmatic advantage, because many drug development activities, including expensive and lengthy toxicology studies with numerous animals and clinical trials with thousands of patients can be saved by conducting such studies once for either the gel or foam presentation instead of twice (for each presentation).

Application can be, for example, hourly, twelve hourly (e.g., twice daily), daily, alternate-day or intermittent, according to the condition of the patient. For reasons of compliance, less frequent applications, where possible, are preferable, e.g., daily single applications. In certain cases, where prolonged or long term treatment is required, an initial dose is provided, followed by a gradual reduction to a lower maintenance dose, which can be increased if further outbreaks occur.

In one or more embodiments, the initial dose of a tetracycline antibiotic is about 18%, or 17.5%, or 16.5%, or 15.5%, or 14.5%, or 13.5%, or 12.5%, or 11.5%, or 10.5%, or 9.5%, or 8.5%, or 7.5%, or 6.5%, or 5.5%, or 4.5%, or 3.5%, or 2.5%, or 1.5%, or 17%, or 16%, or 15%, or 14%, or 13%, or 12%, or 11%, or 10%, or 9%, or 8%, or 7%, or 6%, or 5%, or 4%, or 3%, or 2%, or 1%, or 0.75%, or 0.5%, or 0.25%, or 0.2% by weight of the composition. In one or more embodiments, the maintenance dose of a tetracycline antibiotic is about 7.5% or 6.5% or 5.5% or 4.5% or 3.5% or 2.5% or 1.5% 7% or 6% or 5% or 4% or 3% or 2% or 1% or 0.5% or 1.9% or 1.8% or 1.7% or 1.6% or 1.55 or 1.4% or 1.3% or 1.2% or 1.1% or 0.9% or 0.8% or 0.7% or 0.6% or 0.4% or 0.35 or 0.25% or 0.2% or 0.15% or 0.1% by weight of the composition.

In one or more embodiments, the hydrophobic composition comprises a tetracycline antibiotic. The specific particle size of the tetracycline antibiotic in one or more embodiments is less than or about 25 microns, or less than about 22 microns, or less than about 19 microns, or less than or about 16 microns, or less than or about 13 microns, or less than about 10 microns, or less than or about 9 microns, or less than or about 8 microns, or less than or about 7 microns, or less than or about 6 microns, or less than or about 5 microns. In one or more certain embodiments, 90% of the tetracycline particles are less than or about one of the aforesaid amounts in size. In an embodiment, the particle size ranges from about 6 microns to about 11 microns, or from about 7 microns to about 9 microns or from about 7.5 microns to about 8.5 microns. Skin penetration may be assisted by having a smaller particle size.

In one or more embodiments, such a composition is presented as a breakable gel, which breaks down with mild mechanical force.

In one or more embodiments, the hydrophobic composition, when packaged in an aerosol container to which is added a liquefied or compressed gas propellant, provides upon release from the container a breakable foam of at least good quality that breaks easily upon application of mechanical force.

In one or more embodiments, the composition is a foamable composition that is thermally stable at skin temperature.

In one or more embodiments, when the above composition is filled into an aerosol can and pressurized with a propellant a foamable composition is produced.

In one or more embodiments the carrier is hydrophobic. In some embodiments the carrier comprises or is a hydrophobic solvent. In some embodiments the carrier comprises or is an oil. In some embodiments the carrier comprises or is a liquid. In some embodiments the carrier comprises or is an emollient. In some embodiments the carrier comprises or is a liquid hydrophobic emollient. In some embodiments the carrier comprises or is a liquid wax. In some embodiments the carrier comprises or is a liquid fatty alcohol. In some embodiments the carrier comprises or is a liquid fatty acid.

In one or more embodiments, the carrier is at a concentration of about 40% to about 95% by weight. In one or more embodiments, the carrier is at a concentration of about 42% to about 93% by weight. In one or more embodiments, the carrier is at a concentration of about 44% to about 91% by weight. In one or more embodiments, the carrier is at a concentration of about 50% to about 90% by weight. In one or more embodiments, the carrier is at a concentration of about 55% to about 90% by weight. In one or more embodiments, the carrier is at a concentration of about 60% to about 90% by weight. In one or more embodiments, the carrier is at a concentration of about 65% to about 90% by weight. In one or more embodiments, the carrier is at a concentration of about 70% to about 90% by weight. In one or more embodiments, the carrier is at a concentration of about 75% to about 90% by weight. In one or more embodiments, the carrier is at a concentration of at least about 40% by weight, or at least about 45% by weight, or at least about 50% by weight, or at least about 55% by weight, or at least about 60% by weight, or at least about 65% by weight, or at least about 70% by weight, or at least about 75% by weight, or at least about 80% by weight, or at least about 85% by weight, or at least about 90% by weight, or at least about 92% by weight, or at least about 94% by weight and any ranges between any two figures listed for example from about 55% to about 94%. In some embodiments, the carrier is at a concentration of less than about 95% by weight, i or s at a concentration of less than about 90% by weight, or is at a concentration of less than about 85% by weight, or less than about 80% by weight, or less than about 70% by weight, or less than about 60% by weight, or less than about 50% by weight. In one or more embodiments, the carrier is at a concentration of about 70% by weight, or about 72% by weight, or about 74% by weight, or about 76% by weight, or about 78% by weight, or about 80% by weight, or about 82% by weight, or about 84% by weight, or about 86% by weight, or about 88% by weight, or about 90% by weight, or about 92% by weight, or about 94% by weight, or about 96% by weight, or about 98% by weight. In one or more embodiments, the carrier is at a concentration of about 79.3% by weight, or about 82.4% by weight, or about 87% by weight, or about 87.4% by weight, or about 88% by weight, or about 88.24% by weight, or about 88.6% by weight.

In one or more embodiments, the hydrophobic solvent is at least one hydrophobic solvent. In one or more embodiments the hydrophobic solvent or at least one hydrophobic solvent comprises or is selected from the group consisting of an oil, a mineral oil, a hydrocarbon oil, an ester oil, an ester of a dicarboxylic acid, a triglyceride oil, an oil of plant origin, an oil from animal origin, an unsaturated or polyunsaturated oil, a diglyceride, a PPG alkyl ether, an essential oil, a silicone oil, a liquid paraffin, an isoparaffin, a polyalphaolefin, a polyolefin, a polyisobutylene, a synthetic isoalkane, isohexadecane, isododecane, alkyl benzoate, alkyl octanoate, C₁₂-C₁₅ alkyl benzoate, C₁₂-C₁₅ alkyl octanoate, arachidyl behenate, arachidyl propionate, benzyl laurate, benzyl myristate, benzyl palmitate, bis(octyldodecyl stearoyl) dimer dilinoleate, butyl myristate, butyl stearate, cetearyl ethylhexanoate, cetearyl isononanoate, cetyl acetate, cetyl ethylhexanoate, cetyl lactate, cetyl myristate, cetyl octanoate, cetyl palmitate, cetyl ricinoleate, decyl oleate, diethyleneglycol diethylhexanoate, diethyleneglycol dioctanoate, diethyleneglycol diisononanoate, diethyleneglycol diisononanoate, diethylhexanoate, diethylhexyl adipate, diethylhexyl malate, diethylhexyl succinate, diisopropyl adipate, diisopropyl dimerate, diisopropyl sebacate, diisosteary dimer dilinoleate, diisostearyl fumerate, dioctyl malate, dioctyl sebacate, dodecyl oleate, ethylhexyl palmitate, ester derivatives of lanolic acid, ethylhexyl cocoate, ethylhexyl ethylhexanoate, ethylhexyl hydroxystarate, ethylhexyl isononanoate, ethylhexyl palmytate, ethylhexyl pelargonate, ethylhexyl stearate, hexadecyl stearate, hexyl laurate, isoamyl laurate, isocetyl behenate, isocetyl lanolate, isocetyl palmitate, isocetyl stearate, isocetyl salicylate, isocetyl stearate, isocetyl stearoyl stearate, isocetearyl octanoate, isodecyl ethylhexanoate, isodecyl isononanoate, isodecyl oleate, isononyl isononanoate, isodecyl oleate, isohexyl decanoate, isononyl octanoate, isopropyl isostearate, isopropyl lanolate, isopropyl laurate, isopropyl myristate, isopropyl palmitate, isopropyl stearate, isostearyl behenate, isosteary citrate, isostearyl erucate, isostearyl glycolate, isostearyl isononanoate, isostearyl isostearate, isostearyl lactate, isostearyl linoleate, isostearyl linolenate, isostearyl malate, isostearyl neopentanoate, isostearyl palmitate, isosteary salicylate, isosteary tartarate, isotridecyl isononanoate, isotridecyl isononanoate, lauryl lactate, myristyl lactate, myristyl myristate, myristyl neopentanoate, myristyl propionate, octyldodecyl myristate, neopentylglycol dicaprate, octyl dodecanol, octyl stearate, octyl palmitate, octyldodecyl behenate, octyldodecyl hydroxystearate, octyldodecyl myristate, octyldodecyl stearoyl stearate, oleyl erucate, oleyl lactate, oleyl oleate, propyl myristate, propylene glycol myristyl ether acetate, propylene glycol dicaprate, propylene glycol dicaprylate, propylene glycol dicaprylate, maleated soybean oil, stearyl caprate, stearyl heptanoate, stearyl propionate, tocopheryl acetate, tocopheryl linoleate, glyceryl oleate, tridecyl ethylhexanoate, tridecyl isononanoate, triisocetyl citrate, alexandria laurel tree oil, an avocado oil, an apricot stone oil, a barley oil, a borage seed oil, a calendula oil, a canelle nut tree oil, a canola oil, a caprylic/capric a triglyceride castor oil, a coconut oil, a corn oil, a cotton oil, a cottonseed oil, an evening primrose oil, a flaxseed oil, a groundnut oil, a hazelnut oil, glycereth triacetate, glycerol triheptanoate, glyceryl trioctanoate, glyceryl triundecanoate, a hempseed oil, a jojoba oil, a lucerne oil, a maize germ oil, a marrow oil, a millet oil, a neopentylglycol dicaprylate/dicaprate, an olive oil, a palm oil, a passionflower oil, pentaerythrityl tetrastearate, a poppy oil, propylene glycol ricinoleate, a rapeseed oil, a rye oil, a safflower oil, a sesame oil, a shea butter, a soya oil, a soybean oil, a sweet almond oil, a sunflower oil, a sysymbrium oil, a syzigium aromaticum oil, a tea tree oil, a walnut oil, wheat germ glycerides, a wheat germ oil, PPG-2 butyl ether, PPG-4 butyl ether, PPG-5 butyl ether, PPG-9 butyl ether, PPG-12 butyl ether, PPG-14 butyl ether, PPG-15 butyl ether, PPG-15 stearyl ether, PPG-16 butyl ether, PPG-17 butyl ether, PPG-18 butyl ether, PPG-20 butyl ether, PPG-22 butyl ether, PPG-24 butyl ether, PPG-26 butyl ether, PPG-30 butyl ether, PPG-33 butyl ether, PPG-40 butyl ether, PPG-52 butyl ether, PPG-53 butyl ether, PPG-10 cetyl ether, PPG-28 cetyl ether, PPG-30 cetyl ether, PPG-50 cetyl ether, PPG-30 isocetyl ether, PPG-4 lauryl ether, PPG-7 lauryl ether, PPG-2 methyl ether, PPG-3 methyl ether, PPG-3 myristyl ether, PPG-4 myristyl ether, PPG-10 oleyl ether, PPG-20 oleyl ether, PPG-23 oleyl ether, PPG-30 oleyl ether, PPG-37 oleyl ether, PPG-40 butyl ether, PPG-50 oleyl ether, PPG-11 stearyl ether, a herring oil, a cod-liver oil, a salmon oil, a cyclomethicone, a dimethyl polysiloxane, a dimethicone, an epoxy-modified silicone oil, a fatty acid-modified silicone oil, a fluoro group-modified silicone oil, a methylphenylpolysiloxane, phenyl trimethicone, a polyether group-modified silicone oil and mixtures of any two or more thereof. In some embodiments, the hydrophobic solvent comprises or is selected from the group consisting of a soybean oil, a coconut oil, a cyclomethicone, a light mineral oil, a heavy mineral oil and mixtures thereof. In one or more embodiments, the solvent is tested individually for compatibility with a tetracycline antibiotic and is only used if it passes a compatibility test as described below in the Methods.

In one or more embodiments, the hydrophobic solvent is at a concentration of about 75% to about 90% by weight. In one or more embodiments, the hydrophobic solvent is at a concentration of about 55% to about 90% by weight. In one or more embodiments, the hydrophobic solvent is at a concentration of about 50% to about 90% by weight. In one or more embodiments, the hydrophobic solvent is at a concentration of at least about 40% by weight, at least about 45% by weight, at least about 50% by weight, at least about 55% by weight, at least about 60% by weight, at least about 65% by weight, at least about 70% by weight, at least about 75% by weight, at least about 80% by weight, at least about 85% by weight, at least about 90% by weight at least about 92% by weight, or at least about 94% by weight and any ranges between any two figures listed for example from about 55% to about 94%. In some embodiments, the hydrophobic solvent is at a concentration of less than about 90% by weight, less than about 80% by weight, less than about 70% by weight, less than about 60% by weight, less than about 50% by weight. In one or more embodiments, the hydrophobic solvent is at a concentration of about 70% by weight, or about 72% by weight, or about 74% by weight, or about 76% by weight, or about 78% by weight, or about 80% by weight, or about 82% by weight, or about 84% by weight, or about 86% by weight, or about 88% by weight, or about 90% by weight, or about 92% by weight, or about 94% by weight, or about 96% by weight, or about 98% by weight. In one or more embodiments, the hydrophobic solvent is at a concentration of about 79.3% by weight, or about 82.4% by weight, or about 87% by weight, or about 87.4% by weight, or about 88% by weight, or about 88.24% by weight, or about 88.6% by weight.

In one or more embodiments, the hydrophobic composition comprises a gelled oil. In one or more embodiments, the gelled oil is a gelled mineral oil. In one or more embodiments, the gelled mineral oil is a VERSAGEL®. VERSAGELs® are gelled oils or emollients that can come in different product forms including, for example, the VERSAGEL® m, VERSAGEL® p, VERSAGEL® r and VERSAGEL® s series, and provide various viscosity grades. There are also VERSAGELs® with isohexadecane, or with isododecane, or with hydrogenated polyisobutene, or with isopropylpalmitate. In an embodiment, it is VERSAGEL® 750 m. In an embodiment, it is VERSAGEL® 200 m. In an embodiment, it is VERSAGEL® 500 m. In an embodiment, it is VERSAGEL® 1600 m. VERSAGEL® m contains a mixture of mineral oil plus one or two or more of e.g., Ethylene/Propylene/Styrene Copolymer plus e.g., Butylene/Ethylene/Styrene Copolymer plus e.g., butylated hydroxyl toluene or similar gelling agents. In one or more embodiments, the gelled oil is at a concentration of about 55% to about 85% by weight. In one or more embodiments, the gelled oil is at a concentration of about 60% to about 80% by weight. In one or more embodiments, gelled oil is at a concentration of about 65% to about 75% by weight. In one or more embodiments, the hydrophobic solvent is at a concentration of about 75% to about 90% by weight. In one or more embodiments, the hydrophobic solvent is at a concentration of about 21% to about 39% by weight. In one or more embodiments, the hydrophobic solvent is at a concentration of about 26% to about 34% by weight. In one or more embodiments, the hydrophobic solvent is at a concentration of about 9% to about 24% by weight. In one or more embodiments, the hydrophobic solvent comprises a petrolatum at a concentration of about 9% to about 24% by weight, or about 26% to about 34% by weight or about 21% to about 39% by weight, or about 45% by weight, or about 50% by weight or about 55% by weight or about 60% by weight.

In one or more embodiments, the viscosity-modifying agent is at a concentration of about 0.1% to about 22%, about 0.4 to about 18%, about 0.5% to 16%, about 0.6% to 14%, about 0.7% to 13%, about 0.8 to about 12%, about 0.9% to about 11%, about 1% to about 10%, about 10% to about 22% by weight or a range comprising about any one of the aforesaid lower amounts to about any one of the aforesaid higher amounts, such as, about 0.4% to about 22%. In one or more embodiments, the viscosity-modifying agent is a fatty alcohol having at least 12 carbon atoms in its carbon backbone. In one or more embodiments, the viscosity-modifying agent is a fatty acid having at least 12 carbon atoms in its carbon backbone.

In one or more embodiments, the viscosity-modifying agent is at a concentration of about 9.5% or about 8.5% or about 7.5% or about 6.5% or about 5.5% or about 4.5% or about 3.5% or about 2.5% or about 1.5%, about 7% or about 6% or about 5% or about 4% or about 3% or about 2% or about 1% or about 0.5%, or about 1.9%, or about 1.8%, or about 1.7%, or about 1.6%, or about 1.55 or about 1.4% or about 1.3% or about 1.2% or about 1.1%, or about 0.9% or about 0.8%, or about 0.7%, or about 0.6% or about 0.5% by weight of the composition or less than any of the aforesaid amounts.

In one or more embodiments, the concentration of the hydrophobic solvent, and/or viscosity-modifying agent in the composition is selected to provide an Aw value selected from the ranges between or of (1) about 0.8 and about 0.9; (2) about 0.7 and about 0.8; and (3) less than about 0.7. Delivering the formulation in a pressurized package does not allow for humidity to be absorbed by the preparation, and therefore, the water free character of the composition is not altered.

In one or more embodiments, the emollient comprises or is selected from the group consisting of isostearic acid derivatives, isopropyl palmitate, lanolin oil, diisopropyl dimerate, diisopropyl adipate, dimethyl isosorbide, maleated soybean oil, octyl palmitate, isopropyl isostearate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, hydrogenated coco-glycerides, isononyl isononanoate, isotridecyl isononanoate, myristyl myristate, triisocetyl citrate, octyl dodecanol, octyl hydroxystearate and mixtures thereof. Other examples of other suitable emollients can also be found in the Cosmetic Bench Reference, pp. 1.19-1.22 (1996), which is incorporated herein by reference for emollients.

In one or more embodiments, the fatty alcohol and/or fatty acid have a melting point of at least about 40° C.

In one or more embodiments, the fatty alcohol comprises or is selected from the group consisting of lauryl alcohol, myristyl alcohol, cetyl alcohol, stearyl alcohol, arachidyl alcohol, behenyl alcohol, tetracosanol, hexacosanol, octacosanol, triacontanol, and tetratriacontanol. In one or more embodiments, the fatty acid comprises or is selected from the group consisting of dodecanoic acid, tetradecanoic acid, hexadecanoic acid, heptadecanoic acid, octadecanoic acid, eicosanoic acid, docosanoic acid, tetracosanoic acid, hexacosanoic acid, heptacosanoic acid, octacosanoic acid, triacontanoic acid, dotriacontanoic acid, tritriacontanoic acid, tetratriacontanoic acid, and pentatriacontanoic acid.

In one or more embodiments, the fatty alcohol is about 3% to about 10% by weight. For example, about 3% by weight, or about 4% by weight, or about 5% by weight, or about 6% by weight, or about 7% by weight, or about 8% by weight, or about 9% by weight, or about 10% by weight. For example, about 4.1% by weight, or about 4.4% by weight, or about 4.5% by weight, or about 5% by weight, or about 5.6% by weight, or about 8.6% by weight.

In one or more embodiments, the fatty alcohol is less than about 8% by weight. For example, less than about 7% by weight, or less than about 6% by weight, or less than about 5% by weight, or less than about 4% by weight.

In one or more embodiments, the carbon chain of the fatty alcohol or the fatty acid is substituted with a hydroxyl group.

In one or more embodiments, the fatty acid is 12-hydroxy stearic acid.

In one or more embodiments, the viscosity-modifying agent is a wax comprising or selected from the group consisting of a plant wax, carnauba wax, candelilla wax, ouricury wax, sugarcane wax, retamo wax, jojoba oil, an animal waxes, beeswax, a petroleum derived wax, a paraffin wax, polyethylene, and derivatives thereof.

In one or more embodiments, the viscosity-modifying agent is a combination comprising (i) at least one fatty alcohol and at least one fatty acid; or (ii) at least one fatty alcohol and at least one wax; or (iii) at least one fatty acid and at least one wax; or (iv) at least one fatty alcohol, at least one fatty acid, and at least one wax.

In one or more embodiments, the at least one viscosity-modifying agent comprises or is selected from the group consisting of a fatty alcohol, a fatty acid and a wax, wherein the fatty alcohols and/or fatty acids have at least 12 carbon atoms in their carbon backbone. In certain embodiments the viscosity modifying agent is a combination of a fatty alcohol and a fatty acid and/or a wax.

In some embodiments, the fatty alcohol and/or fatty acid and/or wax are solid at ambient temperature. In certain embodiments, the fatty alcohol and/or the fatty acid and/or the wax or the mixture of them have a melting point of more than about 40° C.

In one or more embodiments, the wax is about 0% to about 6% by weight. For example, about 1% by weight, or about 2% by weight, or about 3% by weight, or about 4% by weight, or about 5% by weight, or about 6% by weight. In one or more embodiments, the wax is about 0.2% by weight.

In one or more embodiments, the wax is less than about 4% by weight. For example, less than about 3% by weight, or less than about 2% by weight, or less than about 1% by weight, or less than about 0.5% by weight.

In one or more embodiments, the fatty acid is about 1% to about 10% by weight. For example, about 1% by weight, or about 2% by weight, or about 3% by weight, or about 4% by weight, or about 5% by weight, or about 6% by weight, or about 7% by weight, or about 8% by weight, or about 9% by weight, or about 10% by weight. For example, about 2.4% by weight, or about 2.5% by weight, or about 3% by weight.

In one or more embodiments, the total amount of fatty acid fatty alcohol and wax, if present is about 1% to about 10% by weight. For example, about 1% by weight, or about 2% by weight, or about 3% by weight, or about 4% by weight, or about 5% by weight, or about 6% by weight, or about 7% by weight, or about 8% by weight, or about 9% by weight, or about 10% by weight. For example, about 2.4% by weight, or about 2.5% by weight, or about 3% by weight.

### Incompatible Excipients and Undesirable Excipients

In certain embodiments, the composition is free of one or more of a petrolatum, surface active agents, protic solvents, certain polar aprotic solvents, isopropyl myristate, polyethylene gelling agents, polyethylene homopolymers, polyethylene copolymers, selenium derivatives and silicone thickening agents. In certain embodiments, the foamable composition is substantially free of such excipients, i.e., the composition contains a total of less than about 0.4% by weight of one or more of a petrolatum, surface active agents, protic solvents, certain polar aprotic solvents, isopropyl myristate, polyethylene gelling agents, polyethylene homopolymers, polyethylene copolymers, selenium derivatives and silicone thickening agents cumulatively. In one or more embodiments, the composition comprises less than about 0.35%, or less than about 0.3%, or less than about 0.25%, or less than about 0.2%, or less than about 0.15%, or less than about 0.1% or less than 0.05% by weight of one or more of petrolatum, surface active agents, protic solvents, certain polar aprotic solvents, isopropyl myristate, polyethylene gelling agents, polyethylene homopolymers, polyethylene copolymers, selenium derivatives, silicone thickening agents, and any combination of two or more thereof cumulatively or, in another embodiment, less than about 0.01% individually or of two or more or all thereof cumulatively.

### Surface Active Agents

In the context herein, the terms “standard surfactant” or “customary surfactant” refer to customary non-ionic, anionic, cationic, zwitterionic, amphoteric, and amphiphilic surfactants. A fatty alcohol or a fatty acid and certain waxes are not regarded as a standard or customary surfactant. However, in contrast, ethers or esters formed from such fatty alcohols or fatty acids can be regarded as a customary or standard surfactant.

Surfactants of all kinds are undesirable in accordance with the compositions, formulations, and methods provided herein, as (i) they were found to cause degradation of the tetracycline antibiotic; and (ii) they are generally known to possess irritation potential.

Non-limiting examples of classes of non-ionic surfactants that are undesirable include: (i) polyoxyethylene sorbitan esters (polysorbates), such as polysorbate 20, polysorbate 40, polysorbate 60 and polysorbate 80; (ii) sorbitan esters, such as sorbitan monolaurate and sorbitan monooleate; (iii) polyoxyethylene fatty acid esters, such as, PEG-8 stearate, PEG-20 stearate, PEG-40 stearate, PEG-100 stearate, PEG-150 distearate, PEG-8 laurate, PEG-10 laurate, PEG-12 laurate, PEG-20 laurate, PEG-8 oleate, PEG-9 oleate, PEG-10 oleate, PEG-12 oleate, PEG-15 oleate and PEG-20 oleate; (iv) PEG-fatty acid diesters; (v) polyethylene glycol (PEG) ethers of fatty alcohols; (vi) glycerol esters, such as glyceryl monostearate, glyceryl monolaurate, glyceryl monopalmitate and glyceryl monooleate; (vii) PEG-fatty acid mono- and di-ester mixtures; (viii) polyethylene glycol glycerol fatty acid esters; (ix) propylene glycol fatty acid esters; (x) mono- and diglycerides; (xi) sugar esters (mono-, di- and tri-esters of sucrose with fatty acids) and (xii) PEG alkyl phenols.

As mentioned above, in the context herein, while fatty alcohols, fatty acids, and certain waxes are somewhat amphiphilic, these substances are not effective as stand-alone surfactants that can stabilize an emulsion, let alone foamable emulsion compositions, because of their very weak emulsifying capacity and further due to their weak foaming capacity on their own.

They are occasionally used in a supporting role as co-emulsifiers, i.e., in combination with a standard surfactant but are commonly used as thickeners and have successfully been used as foam adjuvants to assist customary surfactants to boost foam quality and stability. For the purposes of forming an emulsion, they are usually regarded as an oil and thus have a “required” HLB value for the purpose of determining what standard surfactant might be appropriate to use with the oil phase.

Generally, surfactants are known to possess irritation potential. One way to try and reduce or minimize potential irritation and drying of the skin or mucosa due to surfactants and their repeated use, especially when formulations are to be left on the skin or mucosa rather than being washed off, is to use essentially or primarily nonionic surfactants at significant concentrations although aiming for low concentrations, such as, below 5%. The current breakthrough of identifying formulations which produce gels and quality breakable foam yet omitting customary surfactants from a composition may contribute to improved tolerability of such a composition and can be an important advantage. This is especially so when a formulation is to be applied to a very sensitive target site, and particularly so on a repeated basis.

In certain embodiments, the composition is free of customary surfactants, or “surfactant-free” and in certain embodiments the foamable composition is substantially free of customary surfactants, or “substantially surfactant-free”.

In certain embodiments, the composition is free or substantially free of an ionic surfactant. In certain embodiments, the composition is free or substantially free of a zwitterionic surfactant. In certain embodiments, the composition is free or substantially free of a non-ionic surfactant.

### Protic Solvents

Protic solvents, such as short chain alcohols, glycols and glycerin are incompatible with tetracyclines and therefore are undesirable.

### Aprotic Polar Solvents

WO 11/039637, incorporated herein by reference in its entirety, discloses that certain polar aprotic solvents are incompatible with tetracycline antibiotics. Thus, aprotic polar solvents, such as dimethyl sulfoxide (DMSO), dimethylformamide (DMF), acetonitrile, acetone, methyl ethyl ketone, 1,4-dioxane, tetrahydrofuran (THF), N-methylpyrrolidone, pyridine, piperidine, N-methyl-2-pyrrolidone, 1-methyl-2-pyrrolidinone, and azone (1-dodecylazacycloheptan-2-one) are undesirable.

### Silicone Thickening Agents

Silicone thickening agents comprise one or more polysiloxane-derived components. Such polysiloxanes are typically cross-linked and they have rubber-like characteristics, which require their solubilization in an oil, usually a silicone oil. An example of such a silicone thickening agent is ST-Elastomer 10 (Dow Corning), which is a mixture of high molecular weight dimethicone crosspolymer (12%), in cyclopentasiloxane (cyclomethicone, silicone solvent). With reference to bioavailability of an active agent in the skin following topical application, it is conceivable that cross co-polymers will create a non-permeable film which should block skin penetration and therefore, it is undesirable. Further, in the context of a breakable foam, cyclomethicone is known as a defoamer and therefore its presence in high concentrations in the breakable hydrophobic composition is undesirable.

In one or more other specific embodiments, the drug carrier is formulated substantially free of elastomers. In one or more other specific embodiments, the drug carrier is formulated essentially free of elastomers. In one or more other specific embodiments, the drug carrier is formulated substantially free of silicones. In one or more other specific embodiments, the drug carrier is formulated essentially free of silicones. In one or more other specific embodiments, the drug carrier is formulated with less than about 30% by weight of silicones, or less than about 25% by weight of silicones, or less than about 20% by weight of silicones, or less than about 15% by weight of silicones, or less than about 10% by weight of silicones, or less than about 7.5% by weight of silicones, or less than about 5% by weight of silicones or less than about 2% by weight of silicones; or less than about 1% by weight of silicones; or less than about 0.5% by weight of silicones; or about 1% to about 5% by weight of silicones. In one or more other specific embodiments, the drug carrier does not comprise a silicone other than cyclomethicone.

In one or more embodiments, semi-solid hydrophobic oils are a subsidiary component in the composition, for example being present at less than about 45%, at less than about 40%, at less than about 35%, at less than about 30%, at less than about 25%, less than about 20%, less than about 15%, less than about 10%, or less than about 5% by weight of the composition. In one or more alternative embodiments, semi solid oils are omitted.

In some embodiments, the composition can contain a hydrophobic oil and one or more viscosity-modifying agents. In some embodiments, the compositions demonstrate increased viscosity of such oil, and to which when even small amounts of a suspended tetracycline antibiotic are added, a substantial or synergistic increase in the viscosity of the composition can be observed.

### Polyol

The identification of a “polyol”, as used herein, is an organic substance that contains at least two hydroxy groups in its molecular structure. In one or more embodiments, the polyol is a diol (a compound that contains two hydroxy groups in its molecular structure). Examples of diols include propylene glycol (e.g., 1,2-propylene glycol and 1,3-propylene glycol), butanediol (e.g., 1,2-butanediol, 1,3-butanediol, 2,3-butanediol and 1,4-butanediol), butenediol (e.g., 1,3-butenediol and 1,4-butenediol), butynediol, pentanediol (e.g., pentane-1,2-diol, pentane-1,3-diol, pentane-1,4-diol, pentane-1,5-diol, pentane-2,3-diol and pentane-2,4-diol), hexanediol (e.g., hexane-1,6-diol hexane-2,3-diol and hexane-2,56-diol), octanediol (e.g., 1,8-octanediol), neopentyl glycol, 2-methyl-1,3-propanediol, diethylene glycol, triethylene glycol, tetraethylene glycol, dipropylene glycol and dibutylene glycol.

In one or more embodiments, the polyol is a triol (a compound that contains three hydroxy groups in its molecular structure), such as glycerin, butane-1,2,3-triol, butane-1,2,4-triol and hexane-1,2,6-triol.

In one or more embodiments, the polyol is a saccharide. Exemplary saccharides include, but are not limited to, monosaccharides, disaccharides, oligosaccharides, and sugar alcohols.

A monosaccharide is a simple sugar that cannot be hydrolyzed to smaller units. The empirical formula of a monosaccharide is (CH₂O)ₙ and can range in size from trioses (=3) to heptoses (n=7). Exemplary monosaccharide compounds are, e.g., ribose, glucose, fructose, and galactose.

Disaccharides are made up of two monosaccharides joined together, such as sucrose, maltose, and/or lactose.

In one or more embodiments, the polyol is a sugar alcohol (also known as a polyol, polyhydric alcohol, or polyalcohol) or a hydrogenated form of saccharide, whose carbonyl group (aldehyde or ketone, reducing sugar) has been reduced to a primary or secondary hydroxyl group. They are commonly used for replacing sucrose in foodstuffs, often in combination with high intensity artificial sweeteners to counter the low sweetness. Some exemplary sugar alcohols, which are suitable for use according to the present invention are mannitol, sorbitol, xylitol, maltitol, lactitol. (Maltitol and lactitol are not completely hydrogenated compounds—they are a monosaccharide combined with a polyhydric alcohol.) Mixtures of polyols, including (1) at least one polyol comprises or selected from a diol and a triol; and (2) a saccharide are contemplated within the scope of the present disclosure.

According to some embodiments, the composition is polyol free, i.e., the composition does not comprise any amount of polyols.

In other embodiments, the composition is substantially free of polyols and comprises less than about 5% by weight of the final concentration of polyols, or less than 2% by weight, or less than 1% by weight. In some embodiments the composition comprises de minimis amounts of polyols. Where a formulation includes insignificant or de minimis amounts of polyols, such as less than 0.05% by weight, it is considered to be essentially free of them.

In an embodiment, the polyol is linked to a hydrophobic moiety. In the context of the present disclosure, a polyol linked to a hydrophobic moiety is still defined as a “polyol” as long as it still contains two or more free hydroxyl groups.

In an embodiment, the polyol is linked to a hydrophilic moiety. In the context of the present disclosure, a polyol linked to a hydrophilic moiety is still defined as a “polyol” as long as it still contains two or more free hydroxyl groups.

In one or more embodiments, the hydrophobic composition further contains an anti-infective agent, selected from the group of an antibiotic agent, an antibacterial agent, an antifungal agent, an agent that controls yeast, an antiviral agent, and an antiparasitic agent. In one embodiment, the anti-infective agent comprises a tricyclic antibiotic. Not only can combining the anti-infective effect of a hydrophobic composition with an anti-infective agent result in a synergistic effect and consequently higher success rate of the treatment, but the combination with the viscosity modifying agent achieves a formulation in which the active pharmaceutical ingredient is chemically stable and the formulation is physically stable as demonstrated herein in the Examples. Moreover, the use of hydrophobic-based, water-free formulations can maximize the antimicrobial and antiviral potentials of the formulations. Topical delivery can be improved by using a hydrophobic carrier with a hydrophobic API. Storage in sealed, light and airtight canisters can assist in preserving the formulations.

In one or more embodiments, the hydrophobic composition is substantially free of at least one or more selected from the group consisting of surface active agents, protic solvents, polar aprotic solvents, and silicone thickening agents.

In one or more embodiments, the hydrophobic composition is substantially free of at least one or more selected from the group consisting of surface active agents, polymeric gelling agents, polyols, short chain alcohols, and silicone thickening agents.

In one or more embodiments, the hydrophobic composition contains less than about 0.4% by weight of the composition or less than about 0.2% by weight of the composition or less than about 0.1% by weight of the composition of one or a combination of two, three or all of surface active agents, protic solvents, polar aprotic solvents, and silicone thickening agents.

### The Ingredients as Therapeutic Agents

In certain embodiments, a hydrophobic solvent can possess therapeutic properties. For example, some essential oils can kill microorganisms and can be effective in the treatment or prevention of conditions that involve microbial infection, such as bacterial, fungal and viral conditions. Additionally, hydrophobic solvents can be useful for the treatment of conditions which involve damaged skin, such as psoriasis or atopic dermatitis. The combination of a hydrophobic solvent and a therapeutically effective fatty alcohol or fatty acid may afford a beneficial effect in conditions characterized, for example, by infection and/or inflammation.

Fatty alcohols can also possess therapeutic properties. Long chain saturated and mono unsaturated fatty alcohols, e.g., stearyl alcohol, erucyl alcohol, arachidyl alcohol and behenyl alcohol (docosanol) have been reported to possess antiviral, antiinfective, antiproliferative and anti-inflammatory properties (see, e.g., U.S. Pat. No. 4,874,794). Longer chain fatty alcohols, e.g., tetracosanol, hexacosanol, heptacosanol, octacosanol, triacontanol, etc., are also known for their metabolism modifying properties, and tissue energizing properties.

In one or more embodiments, the active agent can be a placebo or a cosmetic agent. The foamable composition is suitable for use in the manufacture of a medicament including a placebo or active agent.

### Combination of Active Agents

Several disorders involve a combination of more than one etiological factor; and therefore, the use of more than one active agent is advantageous. For example, psoriasis involves excessive cell proliferation and inadequate cell differentiation as well as inflammation. Atopic dermatitis involves keratinocyte growth abnormality, skin dryness and inflammation. Bacterial, fungal and viral infections involve pathogen colonization at the affected site and inflammation. Hence, in many cases, the inclusion of a combination of active agents in the pharmaceutical composition can be desirable. Thus, in one or more embodiments, the composition includes at least two active agents, in a therapeutically effective concentration.

In one or more embodiments, a combination of any two or more of an antibacterial, an anti-inflammatory, an antifungal, and an antiviral agent is contemplated.

In one or more embodiments, there is provided a composition in which the composition further comprises at least one additional active agent selected from the group consisting of an antibiotic agent, a steroidal anti-inflammatory agent, an immunosuppressive agent, an immunomodulator, an immunoregulating agent, a hormonal agent, an androgen, an estrogen, a prostaglandin, an antiandrogen agent, a testosterone inhibitor, a dihydrotestosterone inhibitor, antibacterial agent, an antifungal agent, an antiviral agent, an antiparasitic agent, antimicrobial, a retinoid, vitamin A, a vitamin A derivative, vitamin B, a vitamin B derivative, vitamin C, a vitamin C derivative, vitamin D, a vitamin D derivative, vitamin E, a vitamin E derivative, vitamin F, a vitamin F derivative, vitamin K, a vitamin K derivative, a wound healing agent, a disinfectant, an anesthetic, an antiallergic agent, a keratolytic agent, urea, a urea derivative, an alpha hydroxyl acid, lactic acid, glycolic acid, a beta-hydroxy acid, a protein, a peptide, a neuropeptide, an allergen, an immunogenic substance, a haptene, an oxidizing agent, an antioxidant, a dicarboxylic acid, azelaic acid, sebacic acid, adipic acid, fumaric acid, a retinoid, an antiproliferative agent, an anticancer agent, a photodynamic therapy agent, benzoyl chloride, calcium hypochlorite, magnesium hypochlorite, an anti-wrinkle agent, a radical scavenger, a metal, silver, a metal oxide, titanium dioxide, zinc oxide, zirconium oxide, iron oxide, silicone oxide, an organo-metallic compound, and organo-boron compound, an organo-beryllium compound, a tellurium compound, talc, carbon, an anti wrinkle agent, a skin whitening agent, a skin protective agent, a masking agent, an anti-wart agent, a refatting agent, a lubricating agent and mixtures thereof.

In one or more embodiments the addition of at least one additional active agent is optional.

Wherever a specific active agent is used herein, it can be substituted by another form of the same active agent. For example, in one or more embodiments, minocycline hydrochloride can be substituted by another form of minocycline, and likewise in one or more embodiments, doxycycline hyclate can be substituted by another form of doxycycline. The term “form” can include, for example, salts, hydrates, crystals, polymorphs, enantiomers, isomers, ions, complexes, and the like. In one or more embodiments, the active agent can be in the form of a salt, a hydrate, a crystal, one or more polymorphs, one or more enantiomers, an isomer, an ion, a complex, or any other pharmaceutically acceptable form.

In one or more embodiments, a tetracycline antibiotic is the sole active ingredient present in the composition. In one or more embodiments, a minocycline is the sole active ingredient present in the composition. In one or more embodiments, a doxycycline is the sole active ingredient present in the composition. In one or more embodiments, minocycline and doxycycline are used in combination.

In one or more embodiments, a combination of any two or more of a minocycline, doxycycline, tetracycline antibiotic steroids, corticosteroids, vitamin K, topical anesthetics, antipruritic agents, antihistamines, pramoxine, lidocaine, quaternary lidocaine derivatives, quaternary ammonium derivatives of anesthetic drugs, pimecrolimus, tarcolimus, retinoids, and benzoyl peroxide is contemplated.

In one or more embodiments, quaternary ammonium derivatives of anesthetic drugs include, for example, quaternary lidocaine derivatives, N-methyl lidocaine, N,N-dimethyl prilocaine, N,N,N-trimethyl tocainide, N-methyl etidocaine, N-methyl ropivacaine, N-methyl bupivacaine, N-methyl levobupivacaine, N-methyl mepivacaine, QX314, and Q222, and as are described in US2012/0172429, which is incorporated by reference. In one or more embodiments the effect of these quaternary ammonium derivatives of anesthetic drugs is associated with TRPA1, TRPM8, P2X(2/3) or TRPV1 channels and receptors. In one or more embodiments they bind to receptors on the side of the channels which is internal.

In one or more embodiments, a combination of any two or more of a tetracycline, a tetracycline antibiotic, retinoids, and benzoyl peroxide is contemplated.

In one or more embodiments, a combination of any two or more of benzoyl peroxide, antibiotics, tetracycline antibiotic, retinoids, antiseborrheic medications, anti-androgen medications, hormonal treatments, lactic acid, urea, petrolatum, emollients, salicylic acid, alpha hydroxy acid, azelaic acid, nicotinamide, and a keratolytic agent is contemplated.

In one or more embodiments, the tetracycline is in combination with of one or more of an antihistamine, a corticosteroid, doxepin, or adapalene.

In one or more embodiments, the concentration of the additional active agent is in a range between about 0.1% to about 10% by weight (e.g., about 0.1% to about 8% by weight, or about 0.1% to about 5% by weight, or about 0.1% to about 3% by weight, or about 0.1% to about 2% by weight, or about 0.1% to about 1% by weight, or about 0.1% to about 0.75% by weight, or about 0.1% to about 0.5% by weight, or about 0.1% to about 0.25% by weight, or about 0.25% to about 10% by weight, or about 0.5% to about 10% by weight, or about 1% to about 10% by weight, or about 2% to about 10% by weight, or about 4% to about 10% by weight, or about 6% to about 10% by weight, or about 7% to about 10% by weight, or about 8% to about 10% by weight, or about 0.5% to about 2.0% by weight, or about 0.75% to about 1.5% by weight, or about 1% to about 3% by weight, or about 1% to about 4% by weight, or about 2% to about 6% by weight). In some embodiments, the concentration of the additional active agent is at least about 0.05% by weight, or is at least about 0.1% by weight, or at least about 0.5% by weight, or at least about 1% by weight, or at least about 2% by weight, or at least about 4% by weight, or at least about 6% by weight, or at least about 8% by weight or at least about 10% by weight.

In one or more embodiments, the concentration of the retinoid (e.g. adapalene) is in a range between about 0.1% to about 10% by weight (e.g., about 0.1% to about 8% by weight, or about 0.1% to about 5% by weight, or about 0.1% to about 3% by weight, or about 0.1% to about 2% by weight, or about 0.1% to about 1% by weight, or about 0.1% to about 0.75% by weight, or about 0.1% to about 0.5% by weight, or about 0.1% to about 0.25% by weight, or about 0.25% to about 10% by weight, or about 0.5% to about 10% by weight, or about 1% to about 10% by weight, or about 2% to about 10% by weight, or about 4% to about 10% by weight, or about 6% to about 10% by weight, or about 7% to about 10% by weight, or about 8% to about 10% by weight, or about 0.5% to about 2.0% by weight, or about 0.75% to about 1.5% by weight, or about 1% to about 3% by weight, or about 1% to about 4% by weight, or about 2% to about 6% by weight). In some embodiments, the concentration of the additional active agent is at least about 0.05% by weight, or is at least about 0.1% by weight, or at least about 0.5% by weight, or at least about 1% by weight, or at least about 2% by weight, or at least about 4% by weight, or at least about 6% by weight, or at least about 8% by weight or at least about 10% by weight. In some embodiments the concentration is about 0.01%, or about 0.02%, or about 0.03% or about 0.04%, or about 0.05%, about 0.06%, or about 0.08%, or about 0.11% or about 0.13%, or about 0.15% or about 0.17% or about 0.19%, or about 0.21% or about 0.3%, or about 0.4%, or about 0.5%, or about 0.6% or about 0.7%, or about 0.8%, or about 0.9% or can be a range between any two figures listed in this paragraph, such as, 0.05% to about 0.8%.

In one or more embodiments, the tetracycline antibiotic comprises or is selected from the group consisting of a tetracycline, an oxytetracycline, a demeclocycline, a doxycycline, a lymecycline, a meclocycline, a methacycline, a minocycline, a rolitetracycline, a chlorotetracycline, a tigecycline, and any two or more thereof.

In one or more embodiments, the tetracycline antibiotic is hydrophobic.

In one or more embodiments, the Log of the distribution constant of the tetracycline antibiotic at pH 7.0 (buffer/chloroform) is equal to or less than about 0.2.

In one or more embodiments, the tetracycline antibiotic is present in a free base form, a hydrate form, a salt form, a chelate complex form or a coordination complex form.

In one or more embodiments, the tetracycline antibiotic does not comprise a hydroxy group at carbons 5, 6, and 7.

In one or more embodiments, the tetracycline antibiotic comprises or is selected from the group consisting of a minocycline and a doxycycline. In some embodiments, the tetracycline antibiotic is a minocycline in others a doxycycline and in still others both. In some embodiments, the concentration of a tetracycline antibiotic e.g. a minocycline or a doxycycline is in a range between about 0.1% to about 10% by weight (e.g., about 0.1% to about 8% by weight, or about 0.1% to about 5% by weight, or about 0.1% to about 3% by weight, or about 0.1% to about 2% by weight, or about 0.1% to about 1% by weight, or about 0.1% to about 0.75% by weight, or about 0.1% to about 0.5% by weight, or about 0.1% to about 0.25% by weight, or about 0.25% to about 10% by weight, or about 0.5% to about 10% by weight, or about 1% to about 10% by weight, or about 2% to about 10% by weight, or about 4% to about 10% by weight, or about 6% to about 10% by weight, or about 7% to about 10% by weight, or about 8% to about 10% by weight, or about 0.5% to about 2.0% by weight, or about 0.75% to about 1.5% by weight, or about 1% to about 3% by weight, or about 1% to about 4% by weight, or about 2% to about 6% by weight). In some embodiments, the concentration of minocycline or doxycycline is at least about 0.05% by weight, or is at least about 0.1% by weight, or at least about 0.5% by weight, or at least about 1% by weight, or at least about 2% by weight, or at least about 4% by weight, or at least about 6% by weight, or at least about 8% by weight or at least about 10% by weight.

The topical compositions of the present disclosure avoid, reduce, minimize or do not cause adverse effects, which are attributed to oral tetracycline antibiotics. Photosensitivity, for example, is a known side effect of oral minocycline. It is manifested as an exaggerated sunburn reaction on areas of the body exposed to direct sunlight or ultraviolet light, resulting in muddy brown skin discoloration. Use of minocycline over an extended period of time can also lead to skin pigmentation e.g. manifested as blue-gray skin and blue-gray staining in areas of scaring. Tooth staining potential of oral minocycline in adult populations has also been acknowledged in recent literature.

In one embodiment, there is a higher safety margin for tooth staining/discoloration, above the point which is considered safe for oral preparations in the literature, resulting from the lower systemic exposure to tetracyclines in the topical formulation.

In one or more embodiments, a tetracycline antibiotic applied in a composition topically on the skin has a significantly lower systemic concentration than the same antibiotic given orally (for example, in an embodiment, it may result in an approximately 400-500-fold reduction in exposure as demonstrated Example 4). So in one or more embodiments, there is a higher safety margin applying a topical composition to the skin when compared to oral dosing. In certain embodiments applying the tetracycline topically allows their use above the point which is considered safe for oral preparations in the literature to avoid tooth staining/discoloration, resulting from the lower systemic exposure to tetracyclines from the topical composition.

In some embodiments, doxycycline hyclate penetrates better than minocycline HCl and hence the maximum plasma concentrations can be more than those obtained for minocycline HCl.

In some embodiments, doxycycline hyclate penetration is similar to that of minocycline HCl and hence the maximum plasma concentrations can be similar to those obtained for minocycline HCl.

In some embodiments, a (4% Doxycycline foam) PK Study is not dissimilar to that of a (4% Minocycline foam) PK Study. In some embodiments, a (1% or 2% or 3% Doxycycline foam) PK Study is not dissimilar to that of a (1% or 2% or 3%, respectively, Minocycline foam) PK Study.

In some embodiments, absorption is low in a (4% tetracycline antibiotic, e.g., doxycycline foam) PK Study, and a Cₘₐₓ (Day 1) is about 0.2 ng/mL to about 5 ng/mL. For example, the Cₘₐₓ is about 0.2 ng/mL, or about 0.4 ng/mL, or about 0.6 ng/mL, or about 0.8 ng/mL, or about 1 ng/mL, or about 1.2 ng/mL, or about 1.4 ng/mL, or about 1.6 ng/mL, or about 1.8 ng/mL, or about 2 ng/mL, or about 2.2 ng/mL, or about 2.4 ng/mL, or about 2.6 ng/mL, or about 2.8 ng/mL, or about 3 ng/mL, or about 3.2 ng/mL, or about 3.4 ng/mL, or about 3.6 ng/mL, or about 3.8 ng/mL, or about 4 ng/mL, or about 4.2 ng/mL, or about 4.4 ng/mL, or about 4.8 ng/mL, or about 5 ng/mL.

In some embodiments, absorption is low in a (4% tetracycline antibiotic, e.g., doxycycline foam) PK Study, and a Cₘₐₓ (Day 16) is about 0.2 ng/mL to about 12 ng/mL. For example, the Cₘₐₓ is about 0.2 ng/mL, or about 0.4 ng/mL, or about 0.6 ng/mL, or about 0.8 ng/mL, or about 1 ng/mL, or about 1.2 ng/mL, or about 1.4 ng/mL, or about 1.6 ng/mL, or about 1.8 ng/mL, or about 2 ng/mL, or about 2.2 ng/mL, or about 2.4 ng/mL, or about 2.6 ng/mL, or about 2.8 ng/mL, or about 3 ng/mL, or about 3.2 ng/mL, or about 3.4 ng/mL, or about 3.6 ng/mL, or about 3.8 ng/mL, or about 4 ng/mL, or about 4.2 ng/mL, or about 4.4 ng/mL, or about 4.8 ng/mL, or about 5 ng/mL, or about 5.2 ng/mL, or about 5.4 ng/mL, or about 5.6 ng/mL, or about 5.8 ng/mL, or about 6 ng/mL, or about 6.2 ng/mL, or about 6.4 ng/mL, or about 6.6 ng/mL, or about 6.8 ng/mL, or about 7 ng/mL, or about 7.2 ng/mL, or about 7.4 ng/mL, or about 7.6 ng/mL, or about 7.8 ng/mL, or about 8 ng/mL, or about 8.2 ng/mL, or about 8.4 ng/mL, or about 8.6 ng/mL, or about 8.8 ng/mL, or about 9 ng/mL, or about 9.2 ng/mL, or about 9.4 ng/mL, or about 9.6 ng/mL, or about 9.8 ng/mL, or about 10 ng/mL, or about 10.2 ng/mL, or about 10.4 ng/mL, or about 10.6 ng/mL, or about 10.8 ng/mL, or about 11 ng/mL, or about 11.2 ng/mL, or about 11.4 ng/mL, or about 11.6 ng/mL, or about 11.8 ng/mL, or about 12 ng/mL,

In some embodiments, absorption of a (4% tetracycline antibiotic, e.g., doxycycline foam) PK Study is about 800 times to about 50 times lower than the Cₘₐₓ and AUC for the labeled dose of the oral doxycycline (Oracea® 40 mg). For example, is about 800 times lower, or about 750 times lower, or about 700 times lower, or about 650 times lower, or about 600 times lower, or about 550 times lower, or about 500 times lower, or about 450 times lower, or about 400 times lower, or about 350 times lower, or about 300 times lower, or about 250 times lower, or about 200 times lower, or about 150 times lower, or about 100 times lower, or about 50 times lower, than the Cₘₐₓ and AUC for the labeled dose of the oral extended release doxycycline (Oracea® 40 mg).

Surprisingly, Applicants have previously demonstrated, in U.S. application Ser. No. 13/499,475, minimal to no skin pigmentation following rubbing of 4% minocycline foam onto the skin when observed after about 30 seconds. It was demonstrated that no photosensitivity or skin discoloration was noticed following application of 1% or 4% minocycline foam onto the skin once daily for 12 weeks. Similarly, pigmentation was not observed.

In one or more embodiments, the method is useful for treating EGFRI associated rash, including administering topically to a surface having the disorder a hydrophobic composition as described above, wherein:
(a) the at least one hydrophobic solvent comprises or is selected from a group consisting of a soybean oil, a coconut oil, a cyclomethicone, a light mineral oil, a heavy mineral oil and mixtures thereof;
(b) the at least one viscosity modifying agent comprises or is selected from a group consisting of a fatty acid, a fatty alcohol, a wax, a hydrogenated castor oil, and mixtures thereof; and
(c) the tetracycline antibiotic is minocycline, or a salt thereof, such as minocycline HCl.

In one or more embodiments, the method is useful for treating EGFRI associated rash, including administering topically to a surface having the disorder a hydrophobic composition as described above, wherein:
(a) the at least one hydrophobic solvent comprises or is selected from a group consisting of a soybean oil, a coconut oil, a cyclomethicone, a light mineral oil, a heavy mineral oil and mixtures thereof;
(b) the at least one viscosity modifying agent comprises or is selected from a group consisting of a fatty acid, a fatty alcohol, a wax, a hydrogenated castor oil, and mixtures thereof;
(c) the tetracycline antibiotic is minocycline, or a salt thereof, such as minocycline HCl; and
(d) an additional active agent.

In one or more embodiments, the composition further comprises fumed or modified silica (SiO₂) such as Aerosil R972.

In one or more embodiments, the tetracycline antibiotic is micronized. In an embodiment it is a micronized minocycline. In an embodiment it is a micronized doxycycline.

In one or more embodiments, the active agent is not micronized.

In one or more embodiments, the active agent is micronized so that the diameter of 90% of the particles (d (0.9)), is less than about 30 microns, or less than about 20 microns, or less than about 10 microns. For example, less than about 28 microns, less than about 26 microns, less than about 24 microns, less than about 22 microns, less than about 20 microns, less than about 18 microns, less than about 16 microns, less than about 14 microns, less than about 12 microns, less than about 10 microns, less than about 8 microns, less than about 6 microns, less than about 4 microns, or less than about 2 microns. In some embodiments the average size of the micronized particles is about 30 microns to about 0.5 microns or about 25 microns to about 1 microns or about 20 microns to about 2 microns or about 15 microns to about 3 microns or about 12 microns to about 3.5 microns or about 10 microns to about 4 microns or about 9 microns to about 4.5 microns or about 8 microns to about 5 microns or about 7 microns to about 5.5 microns or a range between any two of the aforesaid amounts, such as about 12 microns to about 5 microns.

In one or more embodiments, the composition is a foamable composition, and further comprises a propellant. Any compatible propellant may be used. In one or more embodiments, the propellant is a gas at room temperature under normal pressure and which may be liquefied at increased pressure at room temperature. Examples of propellants include, without limitation, hydrocarbon propellants such as butane, propane, isobutane, dimethyl ether, fluorocarbons such as 1,1,1,2 tetrafluorethane (Dymel 134), and 1,1,1,2,3,3,3 heptafluoropropane (Dymel 227), and mixtures thereof. In one or more embodiments, a hydrocarbon mixture AP-70 (a mixture of about 30% w/w butane, 20% w/w isobutane and 50% w/w propane) is used.

In one or more embodiments, there is disclosed a method for treating EGFRI associated rash, including administering topically to a surface having the disorder a hydrophobic composition substantially free of surfactants, and/or substantially free of surfactants and polymeric agents as described above, wherein:
(a) the at least one hydrophobic solvent comprises or is selected from a group consisting of a soybean oil, a coconut oil, a cyclomethicone, a light mineral oil, a heavy mineral oil and mixtures thereof,
(b) the fatty alcohol comprises or is selected from a group consisting of cetostearyl alcohol, myristyl alcohol, stearyl alcohol, behenyl alcohol, and mixtures thereof;
(c) the fatty acid comprises or is selected from the group consisting of stearic acid, beeswax, a hydrogenated castor oil, and mixtures thereof;
(d) the wax comprises or is selected from the group consisting of beeswax, a hydrogenated castor oil, a paraffin wax and mixtures thereof; and
(e) the tetracycline antibiotic is selected from a group consisting of a minocycline and a doxycycline.

In one or more embodiments, there is disclosed a method for treating EGFRI associated rash, including administering topically to a surface having the disorder a hydrophobic composition substantially free of surfactants, and/or substantially free of surfactants and polymeric agents as described above, wherein:
(a) the at least one hydrophobic solvent comprises or is selected from a group consisting of a soybean oil, a coconut oil, a cyclomethicone, a light mineral oil, a heavy mineral oil and mixtures thereof;
(b) the fatty alcohol comprises or is selected from a group consisting of cetostearyl alcohol, myristyl alcohol, stearyl alcohol, behenyl alcohol, and mixtures thereof;
(c) the fatty acid comprises or is selected from the group consisting of stearic acid, beeswax, a hydrogenated castor oil, and mixtures thereof;
(d) the wax comprises or is selected from the group consisting of beeswax, a hydrogenated castor oil, a paraffin wax and mixtures thereof;
(e) the tetracycline antibiotic is selected from a group consisting of a minocycline and a doxycycline; and
(f) the composition comprises an additional active agent.

In one or more embodiments, the above hydrophobic composition or any other composition described herein is used to treat one or more of a EGFRI associated rash or rash related symptoms, a tetracycline antibiotic responsive EGFRI associated rash related disorder, an EGFRI associated tetracycline antibiotic responsive skin disorder, an EGFRI associated skin disorder caused by a bacteria, an EGFRI associated tetracycline antibiotic responsive disorder, an EGFRI associated sebaceous gland disorder, and other EGFRI associated superficial infections, including skin infections, when the EGFRI is applied either on its own or simultaneously, consecutively or overlapping with exposure to radiation and or another active pharmaceutical agent.

In one or more embodiments, the above hydrophobic composition or any other composition described herein is used to treat one or more of EGFRI associated rash, EGFRI associated rash related symptoms, a tetracycline antibiotic responsive EGFRI associated rash related disorder, a tetracycline antibiotic responsive skin disorder, skin disorder caused by a bacteria, a tetracycline antibiotic responsive disorder, a sebaceous gland disorder, and other superficial infections, including skin infections.

In one or more embodiments, the tetracycline antibiotic is minocycline HCl.

In one or more embodiments, the tetracycline antibiotic is doxycycline hyclate.

In one or more embodiments, there is provided a hydrophobic foam composition is obtainable from a foamable composition for use in any of the methods described herein, wherein the foamable composition comprises a carrier and a liquefied or compressed gas propellant, wherein the carrier comprises:
about 60% to about 95% by weight of the carrier at least one hydrophobic solvent;
a wax selected from the group consisting of a beeswax, a hydrogenated castor oil, a paraffin wax, a wax that is solid at room temperature, and mixtures of any two or more thereof;
a fatty alcohol, having a carbon chain length of 14 to 22 carbons, a fatty acid having a carbon chain length of 12 to 28 carbons, and mixtures of any two or more thereof; and
a therapeutically effective amount of a tetracycline antibiotic.

In one or more embodiments, there is provided a hydrophobic foam composition is obtainable from a foamable composition for use in any of the methods described herein, wherein the foamable composition comprises a carrier and a liquefied or compressed gas propellant, wherein the carrier comprises:
about 60% to about 95% by weight of the carrier of at least one hydrophobic solvent;
a wax selected from the group consisting of a beeswax, a hydrogenated castor oil, a paraffin wax, a wax that is solid at room temperature, and mixtures of any two or more thereof;
a fatty alcohol, having a carbon chain length of 14 to 22 carbons, a fatty acid having a carbon chain length of 12 to 28 carbons, and mixtures of any two or more thereof;
a therapeutically effective amount of a tetracycline antibiotic; and
an additional active agent.

In one or more embodiments the ratio of carrier to propellant is from about 100:3 to about 100:30.

In one or more embodiments the wax comprises a hydrogenated castor oil, a beeswax, a paraffin wax and mixtures of any two or more thereof.

Also provided herein is a method for treating human skin diseases especially for the treatment of EGFRI associated rash, including administering topically to a surface having the disorder a hydrophobic composition containing:
(a) a mixture of soybean oil in an amount of about 50 weight percent, coconut oil in an amount of about 24 weight percent, cyclomethicone in an amount of about 5 weight percent, and light mineral oil in an amount of about 4 weight percent;
(b) a mixture of about 3.5 weight percent cetostearyl alcohol, about 2.5 weight percent myristyl alcohol, about 1.5 weight percent stearyl alcohol, about 1 weight percent behenyl alcohol, about 3 weight percent stearic acid, about 2 weight percent beeswax, and about 2 weight percent hydrogenated castor oil;
(c) optionally fumed (modified) silica in an amount of about 0.25 weight percent; and
(d) minocycline HCl in an amount of about 1.0 weight percent.

In one or more embodiments, there is provided a hydrophobic foam composition for use in any of the methods described herein comprising:

about 48% to about 51% by weight of soybean oil;

about 23% to about 25% by weight of coconut oil;

about 4% to about 6% by weight of cyclomethicone;

about 0.7% to about 5.5% by weight of light mineral oil;

about 3% to about 4% by weight of cetostearyl alcohol;

about 2% to about 4% by weight of stearic acid;

about 2% to about 3% by weight of myristyl alcohol;

about 1% to about 3% by weight of hydrogenated castor oil;

about 1% to about 3% by weight of beeswax;

about 1% to about 2% by weight of stearyl alcohol;

about 0.5% to about 1.5% by weight of behenyl alcohol; and

about 1% to about 4% by weight of doxycycline hyclate.

In one or more embodiments, a method for treating human skin disorders or diseases is provided. In one or more embodiments, a method of treating one or more of EGFRI associated rash, an EGFRI associated rash related symptoms, a tetracycline antibiotic responsive EGFRI associated rash related disorder, an EGFRI associated tetracycline antibiotic responsive skin disorder, an EGFRI associated skin disorder caused by a bacteria, an EGFRI associated tetracycline antibiotic responsive disorder, an EGFRI associated sebaceous gland disorder, P. EGFRI associated rash bacteria associated disorders and superficial infections, including skin infections, including administering topically to a surface having the disorder a hydrophobic composition substantially free of surfactants, and/or substantially free of surfactants and polymeric agents as described above, containing:

(a) a mixture of soybean oil in an amount of about 50 weight percent, coconut oil in an amount of about 23.6 weight percent, cyclomethicone in an amount of about 5 weight percent, and light mineral oil in an amount of about 1 to about 5% weight percent;

(b) a mixture of about 3.5 weight percent cetostearyl alcohol, about 2.5 weight percent myristyl alcohol, about 1.5 weight percent stearyl alcohol, about 1 weight percent behenyl alcohol, about 3 weight percent stearic acid, about 2 weight percent beeswax, and about 2 weight percent hydrogenated castor oil;

(c) optionally modified (fumed) silica (Aerosil R 972) in an amount of about 0.25 weight percent; and

(d) minocycline HCl (micronized) in an amount of about 1% to about 5 weight percent (e.g., 4.44% weight percent).

In one or more embodiments, any composition described herein can also contain a fragrance. In one or more embodiments, the fragrance is at a concentration of about 0.1% by weight to about 1% by weight.

In one or more embodiments, the composition comprises about 48% w/w to about 51% w/w of soybean oil. In one or more embodiments, the composition comprises about 23% w/w to about 24% w/w of coconut oil. In one or more embodiments, the composition comprises about 4% w/w to about 6% w/w of cyclomethicone. In one or more embodiments, the composition comprises about 1% w/w to about 5% w/w of light mineral oil.

In one or more embodiments, the composition comprises about 3% w/w to about 4% w/w of cetostearyl alcohol. In one or more embodiments, the composition comprises about 2% w/w to about 4% w/w of stearic acid. In one or more embodiments, the composition comprises about 2% w/w to about 3% w/w of myristyl alcohol. In one or more embodiments, the composition comprises about 1% w/w to about 2% w/w of stearyl alcohol. In one or more embodiments, the composition comprises about 0.5% w/w to about 1.5% w/w of behenyl alcohol. In one or more embodiments, the composition comprises about 1% w/w to about 3% w/w of hydrogenated castor oil. In one or more embodiments, the composition comprises about 1% w/w to about 3% w/w of beeswax.

In one or more embodiments, the composition comprises about 0.1% w/w to about 0.3% w/w of fumed (modified) silica.

In one or more embodiments, the composition comprises about 1% w/w to about 4% w/w of minocycline hydrochloride or a doxycycline or a tetracycline antibiotic.

In one or more embodiments, the composition comprises about 3% w/w to about 15% w/w of propellant based on the weight of the total composition.

In one or more embodiments, there is provided a method for treating EGFRI associated rash, including administering topically to a surface having the disorder a composition which is highly effective against bacteria. In one or more embodiments, the tetracycline antibiotic is effective against some multi-drug resistant strains (e.g., antibiotic-resistant P. EGFRI associated rashs).

In one or more embodiments, there is provided a method for treating EGFRI associated rash, including administering topically to a surface having the disorder a composition which is highly effective against antibiotic-resistant P. EGFRI associated rashs bacteria.

In one or more embodiments, there is provided a method for treating EGFRI associated rash, including administering topically, once a day, to a surface having the disorder a composition comprising a tetracycline antibiotic.

In one or more embodiments, there is provided a method for treating EGFRI associated rash, including administering topically, twice a day, to a surface having the disorder a composition comprising a tetracycline antibiotic.

In one or more embodiments, there is provided a method for treating EGFRI associated rash, including administering topically, alternate-day or intermittently, to a surface having the disorder a composition comprising a tetracycline antibiotic.

In one or more embodiments, there is provided a method for treating EGFRI associated rash, including administering topically, gradual reduction to a lower maintenance dose, which can be increased if further outbreaks occur, to a surface having the disorder a composition comprising a tetracycline antibiotic. In one or more embodiments, a maintenance dose can be applied topically, daily, alternate daily, twice weekly or weekly for a month, two months, quarterly, six months or indefinitely. A maintenance dose can include about 0.9%, or about 0.8%, or about 0.7%, or about 0.6%, or about 0.5%, or about 0.4%, or about 0.3%, or about 0.2%, or about 0.1%, or about 0.09%, or about 0.08%, or about 0.07%, or about 0.06%, or about 0.05% by weight of a tetracycline antibiotic.

In one or more embodiments, there is provided a method for treating EGFRI associated rash, including administering topically, once daily for at least six weeks, to a surface having the disorder a composition comprising a tetracycline antibiotic.

In one or more embodiments, there is provided a method for treating EGFRI associated rash, including administering topically, once daily upto six weeks, to a surface having the disorder a composition comprising a tetracycline antibiotic.

In one or more embodiments, there is provided a method for treating EGFRI associated rash, including administering topically, once daily for twelve weeks or less than twelve weeks, to a surface having the disorder a composition comprising a tetracycline antibiotic.

In one or more embodiments, there is provided a method for treating EGFRI associated rash, including administering topically, once daily for six weeks or less than six weeks, to a surface having the disorder a composition comprising a tetracycline antibiotic.

The compositions provided herein are manufactured according to the methods described in the art and as described in Example 1. Gels are usually packaged in a tube but can also be packaged in any other convenient delivery form including for example, bottles with a pump mechanism or canisters such as bag in can devices where propellant is separate from the gel. Foam formulations are usually packed in a container with an outlet valve. Possible containers and valves are likewise described in the literature as known by those skilled in the art.

In one or more embodiments, the composition is substantially alcohol-free, i.e., free of short chain alcohols having up to 5 carbon atoms in their carbon chain skeleton. In other embodiments, the composition comprises less than about 5% by weight final concentration of short chain alcohols, for example, less than 2% by weight, or less than 1% by weight. In certain embodiments, the composition is free or substantially free of ethanol, propanol, butanol and pentanol.

It is an advantage of the compositions provided herein is that they can be effective when administered once daily for only six weeks. In certain embodiments, the composition may further be effective even if administered alternate-day according to the condition of the patient. In other embodiments, the composition may be used even if administered more than once a day and/or for more than a twelve weeks according to the condition of the patient and the concentration of the minocycline.

A disadvantage of compositions that have an ointment base, comprising petrolatum is that they are greasy and generally considered less usable in the case of facial treatment of EGFRI associated rash. Another disadvantage is where compositions contain surfactants, which can be irritants. In some cases, irritation at the application site has been reported with the use of such compositions.

It is therefore an advantage of the compositions provided herein that they are breakable gels or foams; and therefore are easy to apply to the skin and also avoid skin irritation that has been associated with compositions containing surfactants.

Therapeutic topical compositions must stay on the skin for a sufficient period of time to allow the active agent to be absorbed onto the skin, to perform its activity and to further exert a preventative effect. They should ideally not irritate the skin; and they should be perceived by the patient as pharmaceutically convenient in order to achieve sufficient patient compliance. By “pharmaceutically convenient”, it is meant that the skin look and feel to the patient is good, i.e., it must not be too watery or too greasy and it must easily be applied.

Foam is extremely advantageous in the topical treatment of skin diseases, especially in patients with skin or mucosa afflicted with EGFRI associated rash, especially sensitive skin or mucosa, since it is light and easy to apply and collapses and spreads with a minor mechanical force like a simple rub. When dispensed, even in small quantities, drug delivery in the form of foam can also cover a larger surface area of application while also facilitating better product application in areas where conventional topical products cannot be as effective. Foam absorbs rapidly—without the need of repeated rubbing—which is helpful and important for treatment of damaged or irritated or inflamed skin or mucosa, sores, and lesions.

Thermally stable foam which breaks upon application of mild shear force is extremely advantageous in the topical treatment of skin diseases. It can be applied directly onto skin or hands of the patient without collapsing. So hydrophobic compositions according to the description provided herein, facilitate easy application and even distribution of the active agent, thereby improving treatment convenience. In contrast, Evoclin foam is a temperature sensitive foam that collapses immediately on the skin so it must first be applied onto a cool surface and then quickly applied using fingertips onto the surface which impedes patient compliance.

The formulation packaged into an aerosol container is devoid of any contact with air, light, or any other form of contamination as it is a completely sealed system throughout the life of the product. Thus, light and oxidation sensitive actives can be stabilized effectively in the aerosol system.

In one or more embodiments, there is provided a method of administering a tetracycline foam composition to a target area such as skin of a patient comprising releasing foam, applying it to the area, and collapsing the foam. In one or embodiments, the foam is applied by spreading. In the course of spreading mechanical shear can cause the foam to collapse. In one or more embodiments, the collapsed foam is not washed off. In one or more embodiments, it is absorbed onto the area of skin. In one or more embodiments, it avoids skin irritation or an ointment sensation.

Breakable gels, which comprise liquid oils and a thickening agent, are also very convenient for use, as they liquefy on application of mild shear force such as gentle rubbing, and in turn, they readily absorb onto the skin.

In one or more embodiments, there is provided a method of applying a tetracycline gel composition to an area of skin of a patient comprising releasing a gel, applying it to the area, and collapsing or liquefying the gel. In one or more embodiments, the collapsed or liquefied gel is not washed off. In one or more embodiments, the collapsed or liquefied gel is readily absorbed and does not leave an ointment sensation.

In one or more embodiments, a gel or a liquid gel or a foam or a collapsed foam that is applied to or spread on a skin or mucosal or eye surface is absorbed within 240 seconds, or within 200 seconds, or within 180 seconds, or within 150 seconds, within 120 seconds, or within 100 seconds, or within 80 seconds, or within 60 seconds, or within 50 seconds, or within 40 seconds, or within 30 seconds, or within 20 seconds, or within 10 seconds, or within 5 seconds, or within 2 seconds or less. By absorbed is meant that the composition enters onto and into an area of skin, mucosa or eye, often forming a thin coating on the surface.

In one or more embodiments, there is provided a method for ameliorating anti-tumor induced skin toxicity comprising administrating a tetracycline composition which accelerates wound healing and restores skin integrity.

In one or more embodiments, there is provided a method for ameliorating anti-tumor induced skin toxicity comprising administrating a tetracycline composition which restores normal maintenance functions of skin health.

In one or more embodiments, the normal maintenance functions are selected from the group consisting of control of skin cell differentiation, control of skin cell migration, control of skin cell survival, protection against damage induced by ultraviolet radiation, acceleration of wound healing, and mixtures of any two or more thereof.

In one or more embodiments, there is provided a method for ameliorating anti-tumor induced skin toxicity comprising administrating a tetracycline composition

In one or more embodiments, tetracycline administration inhibits keratinocyte apoptosis.

In one or more embodiments, tetracycline administration restores normal cell cycle in epithelial cells.

In one or more embodiments, there is provided a method for ameliorating anti-tumor induced skin toxicity comprising administrating a tetracycline composition which targets inflammation induced by increased proinflammatory response.

In one or more embodiments, the proinflammatory response involves the proinflammatory agents selected from the group consisting of CCL27, CCL2, CCL5, CCL3, CCL18, CXCL1, CXCL9, CXCL10, CXCL14, IL1, IL6, IL7, NFκB, IRF5, and mixtures of any two or more thereof.

In one or more embodiments, there is provided a method for ameliorating anti-tumor induced skin toxicity comprising administrating a tetracycline composition which targets inflammation induced by inhibition of tumor-induced immune escape mechanisms.

In one or more embodiments, the tumor-induced immune escape is based on a mechanism selected from the group consisting of tumour associated-macrophages (TAMs), regulatory T cells (Treg), myeloid-derived suppressor cells (MDSCs), M2-like macrophages, reduced levels of chemokines that recruit inflammatory immune cells, and mixtures of any two or

In one or more embodiments, the effect of administering a composition comprising a tetracycline antibiotic is to counteract or ameliorate rash like side effects of EGFR inhibitors. In one or more embodiments, the effect of administering a composition comprising a tetracycline antibiotic is achieved by delivering the tetracycline antibiotic onto and into the skin or mucosa or follicles. In one or more embodiments, the effect of administering a composition comprising a tetracycline antibiotic is achieved by delivering the tetracycline through the skin or mucosa or follicles. In one or more embodiments, the effect of administering a composition comprising a tetracycline antibiotic is achieved by delivering the tetracycline onto, into and through the skin or mucosa or follicles. In one or more embodiments, systemic penetration through the skin, mucosa or follicles is low. In one or more embodiments, systemic penetration through the skin, mucosa or follicles is less than about 10%, or about 9%, or about 8%, or about 7%, or about 6%, or about 5%, or about 4%, or about 3%, or about 2%, or about 1%. In one or more embodiments, the average maximum systemic penetration through the skin, mucosa or follicles is less than 5 ng/mL or about 5 ng/mL. In one or more embodiments, the maximum systemic penetration through the skin, mucosa or follicles is between about 1.5 ng/mL to about 6.2 ng/mL. In one or more embodiments, systemic penetration through the skin, mucosa or follicles supplements the effect produced by the non-systemic delivery onto and into the skin, mucosa or follicles without penetrating through the skin, mucosa or follicles. In one or more embodiments, the tetracycline antibiotic may without being bound by any theory act in a way directly or indirectly to e.g. affect EGFR receptors in the skin, mucosa or follicles so as, for example, to help partially or fully to return or restore skin, mucosa or follicle function or cycle to normal.

Successful topical treatment or amelioration (prophylactically or otherwise) of a systemically induced rash is surprising when the source of the rash is systemic. In one or more embodiments, a composition comprising a tetracycline antibiotic is administered topically. In one or more embodiments, the composition is a gel, paste, lotion, cream, soap, spray, mask, patch, powder, pomade, ointment, oil, foam or mousse (expand). In one or more embodiments, the composition is hydrophobic. In one or more embodiments, the composition comprises hydrophobic oils and waxes. In one or more embodiments, the composition comprises fatty alcohols. In one or more embodiments, the composition comprises hydrophobic oils and waxes. In one or more embodiments, the composition comprises fatty acids. In one or more embodiments, the composition is surfactant free. In one or more embodiments, the composition is given as an adjunct to treatment with an EGFR inhibitor. In one or more embodiments, the EGFR inhibitor is an antibody. In one or more embodiments, the antibody is a monoclonal antibody such as cetuximab, or panitumumab, zalutumumab, nimotuzumab, or matuzumab. In one or more embodiments, the inhibitor targets EGFR tyrosine kinase, such as erlotinib, or gefitinib, lapatinib, canertinib or vandetanib. In one or more embodiments, the composition is given prophylactically before onset of EGFR inhibitor therapy. In one or more embodiments, the composition is administered at the beginning of inhibitor therapy. In one or more embodiments, the composition is administered in parallel with inhibitor therapy. In one or more embodiments, the composition is administered after the beginning of inhibitor therapy. In one or more embodiments, the composition is administered during the first week, first two weeks, first three weeks, first month, first five weeks, first six weeks, first seven weeks, first eight weeks, first nine weeks first ten weeks, first eleven weeks or first twelve weeks of inhibitor therapy or some similar period. The term week is used approximately in this context and could include part of a week. In one or more embodiments, the composition is administered one two, three, four five six seven or eight weeks prior to the beginning of inhibitor therapy.

In one or more other embodiments clinical success or improved outcome is determined by increasing the time to develop of a severe rash, thereby avoiding or deferring the time in which dose reduction or treatment interruption takes place.

In one or more other embodiments clinical success or improved outcome is determined by less need to reduce dose.

Minocycline and doxycycline act to reduce the inflammation, thereby reducing EGFRI associated rash severity. Minocycline and doxycycline may also have skin regenerating and healing properties responsible for restoration of skin integrity. The combination of minocycline together with a hydrophobic solvent and a therapeutically effective fatty alcohol or fatty acid may afford a beneficial effect in conditions characterized, for example, by infection and/or inflammation.

In one or more embodiments, there is provided a method for treating EGFRI associated rash, including administering topically, to a surface having the disorder, a composition comprising a tetracycline antibiotic, wherein a reduction in rash severity is observed after five weeks or less than five weeks of treatment compared to baseline. In one or more embodiments, there is provided a method for treating EGFRI associated rash, including administering topically, to a surface having the disorder, a composition comprising a tetracycline antibiotic, wherein an improvement in the skin condition is observed after five weeks or less than five weeks of treatment and wherein an improvement is considered as restoration of visible, normal cutaneous topographic features, indicating the return of skin integrity.

In one or more embodiments, there is provided a method for reducing the severity of EGFRI associated rash severity, by applying topically an effective amount of a tetracycline gel, liquid gel or foam to an afflicted area of a patient in need. In one or more embodiments, the method involves applying a gel, liquid, gel or foam formulation topically to a target surface in need of treatment and breaking the gel or foam over the target site. In one or more embodiments, the method uses an at least once daily dosage regime for twelve weeks or less than twelve weeks. In one or more embodiments, the twelve week dosage regime is followed by an at least once daily maintenance dose for one, two, three or more weeks according to the condition and response of the patient. In one or more embodiments, the method uses an at least once daily dosage regime for six weeks or less than six weeks. In one or more embodiments, the six week dosage regime is followed by a once daily maintenance dose for one, two, three or more weeks according to the condition and response of the patient. In one or more embodiments, the method uses an at least once daily dosage regime of for six weeks or less than six weeks followed by an at least once weekly maintenance dose for one, two, three, four, five, six, seven, eight, nine, ten, eleven and/or more weeks according to the condition and response of the patient. In one or more embodiments, the method uses an at least once daily dosage regime of for three weeks or less than three weeks followed by a once weekly maintenance dose for one, two, three, four, five, six, seven, eight, nine, ten, eleven, twelve or more weeks according to the condition and response of the patient. In one or more embodiments, the method uses a once daily dosage regime of for two weeks followed by a daily maintenance dose for one, two, three or more weeks according to the condition and response of the patient. In one or more embodiments, the method uses a once daily dosage regime of for twelve weeks wherein the treatment is every alternate week.

In one or more embodiments, the method uses an additional step of pre cleaning and drying the rash and surrounding area before applying the gel, liquid gel or foam.

In one or more embodiments, the method uses a sterile applicator or prior to the steps of administering and/or collapsing and/or spreading, the hands of the person spreading are sterilized in order to avoid cross contamination.

In one or more other embodiments, the method uses an additional step of applying an active agent selected from a group consisting of a hyaluronic acid or a retinoid or BPO or salicylic acid, or an alpha hydroxy acid, or azelaic acid, or nicotinamide, or a keratolytic agent or an antipruritic agent, or a quaternary ammonium derivative of an aesthetic drug to the lesions and surrounding area after the gel, liquid gel or foam has been absorbed. In certain embodiments the active agent selected from a group consisting of a hyaluronic acid or a retinoid or BPO or salicylic acid, or an alpha hydroxy acid, or azelaic acid, or nicotinamide, or a keratolytic agent is applied once daily at least 1, or, 2 or 3 or 4 or 5 or 6 or 7 or 8 or 9 or 10 or 11 or 12 hours after the tetracycline antibiotic formulation has been absorbed. In one or more embodiments, the active agent selected from a group consisting of a hyaluronic acid or a retinoid or BPO or salicylic acid, or an alpha hydroxy acid, or azelaic acid, or nicotinamide, or a keratolytic agent is applied after the third day. In one or more embodiments, the active agent selected from a group consisting of a hyaluronic acid or a retinoid or BPO or salicylic acid, or an alpha hydroxy acid, or azelaic acid, or nicotinamide, or a keratolytic agent is applied during the maintenance stage. In an alternative embodiment the active agent selected from a group consisting of a hyaluronic acid or a retinoid or BPO or salicylic acid, or an alpha hydroxy acid, or azelaic acid, or nicotinamide, or a keratolytic agent is replaced with or supplemented by a steroid.

In an alternative embodiment the active agent selected from a group consisting of a hyaluronic acid or a retinoid or BPO or salicylic acid, or an alpha hydroxy acid, or azelaic acid, or nicotinamide, or a keratolytic agent or an antipruritic agent, or a or a quaternary ammonium derivative of an aesthetic drug or steroid is replaced with or supplemented by an antibiotic. In an embodiment, the antibiotic, which is in addition to one or more tetracycline antibiotics, is selected from the group consisting of mupirocin, fusidic acid, a penicillin or penicillin derivative, augmentin, an antistaphylococcal penicillin, amoxicillin/clavulanate, a cephalosporin, cephalexin, a macrolide, erythromycin, clindamycin, trimethoprim-sulfamethoxazole penicillin, retapamulin, and mixtures of any two or more thereof. In an embodiment, the antibiotic is applied topically. In another embodiment it is applied orally or by injection or by infusion. In another embodiment more than one antibiotic is applied. For example, one is applied topically and another is given orally. The latter may be appropriate for example where there is a systemic as well as a topical bacterial infection.

Both the minocycline and the foamable compositions containing minocycline are manufactured under current Good Manufacturing Principles (cGMP) conditions. The foamable composition can be provided in aluminum aerosol canisters mounted with valve and actuator. Each canister can be filled with 25 g of product and 3 g of propellant. Upon actuation of the canister an aliquot of quality foam can be released.

The stability of foamable composition containing doxycycline or minocycline can be monitored at e.g. 5° C., 25° C. and 40° C. and satisfactory stability results are obtained.

A randomized double blind placebo controlled Phase II clinical study, is being conducted in patients afflicted with EGFRI associated rash, and is designed to further assess the efficacy, safety and tolerability of foamable composition comprising doxycycline at concentration of 4% by weight of the formulation, in comparison with placebo. The concentrations of minocycline in the composition are selected according to formulation integrity and stability considerations.

The half face design is selected where each patient serves as his own control in order to properly assess the prophylactic effect of the tetracycline foam. Thus, accurate and careful compliance is crucial for obtaining a successful study; i.e., taking medication on the correct side twice daily, avoiding contaminating the individual sides of face by washing hands between applying sides and/or using a different finger for application, not washing off medication.

### Methods

### Canisters Filling and Crimping

Each aerosol canister is filled with the pre-foam formulation (“PFF”, i.e., foamable carrier) and crimped with valve using vacuum crimping machine. The process of applying a vacuum will cause most of the oxygen present to be eliminated. Addition of hydrocarbon propellant may, without being bound by any theory, further help to reduce the likelihood of any remaining oxygen reacting with the active ingredient. It may do so, without being bound by any theory, by one or more of dissolving in, to the extent present, the oil or hydrophobic phase of the formulation, by competing with some oxygen from the formulation, by diluting out any oxygen, by a tendency of oxygen to occupy the dead space, and by oxygen occupying part of the space created by the vacuum being the unfilled volume of the canister or that remaining oxygen is rendered substantially ineffective in the formulation.

### Pressurizing & Propellant Filling

Pressurizing is carried out using a hydrocarbon gas or gas mixture. Canisters are filled and then warmed for 30 seconds in a warm bath at 50° C. and well shaken immediately thereafter.

### Tests

By way of non-limiting example the objectives are briefly set out below as would be appreciated by a person of skill in the art.

### Collapse Time

Collapse Time, which is the measure of thermal stability, is examined by dispensing a given quantity of foam and photographing sequentially its appearance with time during incubation at 36° C. The collapse time result is defined as the time when the foam height reaches 50% of its initial height or if the foam has not yet reached 50% of its initial height after say 180 seconds then the collapse time is recorded as being >180. By way of illustration, one foam may remain at 100% of its initial height for three minutes, a second foam may reach 90% of its initial height after three minutes, a third foam may reach 70% of its initial height after three minutes, and a fourth foam may reach 51% of its initial height after three minutes, nevertheless in each of these four cases the collapse time is recorded as >180 seconds since for practical purposes for easy application by a patient to a target the majority of the foam remains intact for more than 180 seconds. If the foam, for example, reaches 50% of its original height after say 100 seconds it would be recorded as having a collapse time of 100 seconds. It is useful for evaluating foam products, which maintain structural stability at skin temperature for at least 1 minute. Foams which are structurally stable on the skin for at least one minute are termed “short term stable” carriers or foams.

Alternatively, a Simple Collapse Time can be assessed by placing a foam sample on the warm fingers of a volunteer and measuring the time it takes to melt on the fingers.

### Viscosity

Viscosity is measured with Brookfield LVDV-II+PRO with spindle SC4-25 at ambient temperature and 10, 5 and 1 RPM. Viscosity is usually measured at 10 RPM. However, at about the apparent upper limit for the spindle of ˜>50,000 CP, the viscosity at 1 RPM may be measured, although the figures are of a higher magnitude. Unless otherwise stated, viscosity of the pre-foam formulation (PFF) is provided. It is not practical to try and measure the viscosity of the foamable formulation with regular propellants since they have to be stored in sealed pressurized canisters or bottles. In order to simulate the viscosity in the foamable formulations with propellant an equivalent weight of pentane (a low volatile hydrocarbon) is added to and mixed with the pre-foam formulation and left overnight. The viscosity is then measured as above.

### FTC (Freeze Thaw Cycles)

Foam appearance under extreme conditions of repeated heating and cooling is evaluated by cycling through cooling, heating, (first cycle) cooling, heating (second cycle) etc., conditions, commencing with −10° C. (24 hours) followed by +40° C. (24 hours) and measuring the appearance following each cycle. The cycle is repeated up to three times.

### Chemical Stability

The amount of active agent present is analyzed chromatographically in foam released from various pressurized canisters or in the gel or liquid gel. Analysis is carried out at baseline and at appropriate time intervals thereafter. The canisters are typically stored in controlled temperature incubators at one or more of 5° C., 25° C., 40° C. and 50° C. At appropriate time intervals canisters are removed and the amount of active agent in the foam sample is measured.

### Microbiological Tests

Microbial load: Testing was performed according to EP 2.6.12 and 2.6.13 as described in the European Pharmacopeia.

Preservative efficacy: Testing was performed according to USP <51> and EP 5.6, 2007 5.1.3, as described in the European and US Pharmacopeia.

The test consists of challenging the product with specified microorganisms, storing the inoculated preparations at a prescribed temperature, removing the inoculated samples at specified intervals of time and counting the number of viable organisms in the withdrawn samples using a plate-count procedure. Formulations were challenged by introducing the following microorganisms:

Escherichia coli (ATCC no. 8739)
Staphylococcus aureus (ATCC no. 6538)
Pseudomonas aeruginosa (ATCC no. 9027)
Candida albicans (ATCC no. 10231)
Aspergillus niger (ATCC no. 16404)

The number of colony-forming units (cfu/g) determined at each incubation time point was compared to the number of cfu/g measured in non-inoculated control samples. In order to verify that the samples tested are free of microbial contaminants, the microbial load (base-line) in the samples was determined prior to preservative efficacy testing. Study results are expressed as the number of surviving microorganisms (cfu/g).

Water Activity (Aw): The test for water activity was performed on pre-foam formulation samples introduced into the measuring cell of a PAWKIT water activity meter from DECAGON.

In-vitro effect on microbial growth: The tested microorganism is grown on Tryptic Soy Agar Slants. After incubation, the bacteria is harvested using sterile buffer phosphate pH 7.0, to obtain a microbial count of about 10⁴ cfu/mL. 0.2 mL of the above suspension is spread on Letheen Agar plate and put aside to dry for 20 minutes at room temperature. A sterile disc of 6 mm diameter which has been soaked in 10 μl of the tested antibacterial pre-foam-formulation (PFF) is put on the microbial film, the plate is incubated at 35° C. for 1-2 days. A control experiment is also performed where no antibacterial material is put on the sterile discs. Antimicrobial activity of the tested material inhibits growth of the microorganism around the disc, leaving a transparent zone around it. The diameter of the inhibition zone is measured in mms.

### Compatibility

Active agent is incubated with various excipients individually at one or more temperatures and at different ratios of active agent to a single excipient for a certain fixed period or to the point where degradation was suspected. The period can be for example 3 or 7 or 14 or 21 or 28 days or longer. Visual inspection is a criterion for indication of compatibility. Any change of color indicates oxidation or degradation. For example, the color of an intact MCH suspension is a pale yellow; and a change of color e.g., to dark orange, red, green, brown and black, indicates oxidation or degradation. Tests are also carried out with combinations of excipients.

### Color/Pigmentation

### Part A—Color Change

Samples of formulations are observed and then incubated during 3 months at 25° C., 30° C. and 40° C. Following this period the foam product is actuated and color is observed, and a change, if any, is noted.

### Part B—Pigmentation

Samples are applied to fair healthy human skin to observe whether any skin pigmentation occurs. The skin is observed prior to and 30 seconds following application.

### EXAMPLES

In one or more embodiments, the amounts in the examples should be read with the prefix “about”.

As used herein, the term “NM” refers to not measured.

### Example 1A—General Manufacturing Procedures for a Gel or a Foam

The following procedures were used to produce gel or foam samples, in which only the steps relevant to each formulation were performed depending on the type and nature of ingredients used.

Step 1: Hydrophobic solvents such as mineral oils are mixed at room temperature. Others solvents such as silicones, if present, are added at room temperature under mixing until formulation homogeneity is obtained.

Step 2: The formulation is warmed to 70-80° C. or 80-90° C. and solid compounds such as fatty alcohols, fatty acids and waxes are added and mixed until complete dissolution.

Step 3: The formulation is cooled down to 30-40° C. and active agents such as tetracyclines are added under mixing until formulation homogeneity is obtained.

Step 4: For gel compositions, the formulation is packaged in suitable containers. For foamable compositions, the formulation is packaged in aerosol canisters which are crimped with a valve, pressurized with propellant and equipped with an actuator suitable for foam dispensing. Optionally, a metered dosage unit can is utilized, to achieved delivery of desirable and/or repeatable measured doses of foam.

Step 5: For foamable compositions, pressurizing is carried out using a hydrocarbon gas or gas mixture. Canisters are filled and then warmed for 30 seconds in a warm bath at 50° C. and well shaken immediately thereafter.

Step 6: The canisters or containers are labeled.

### Example 1B—General Manufacturing Procedures for a Gel or a Foam

The following procedures are used to produce gel or foam samples, in which only the steps relevant to each formulation are performed depending on the type and nature of ingredients used.

Step 1: Hydrophobic solvents and solid compounds such as fatty alcohols, fatty acids and waxes are mixed and heated to a temperature sufficient to achieve complete dissolution.

Step 2: The formulation is cooled down to 35-40° C., sensitive components such as cyclomethicone and sensitive active agents such as tetracyclines are added under mixing until formulation homogeneity is obtained.

Step 3: The formulation is cooled down to room temperature.

Step 4: For gel compositions, the formulation is packaged in suitable containers. For foamable compositions, the formulation is packaged in aerosol canisters which are crimped with a valve, pressurized with propellant and equipped with an actuator suitable for foam dispensing.

Step 5: For foamable compositions, pressurizing is carried out using a hydrocarbon gas or gas mixture. The canisters or containers are labeled.

In one or more embodiments, part of the hydrophobic solvents are added during the cooling process of the formulation (step 2).

In one or more embodiments, one of more of the formulation mixing steps may be done with or without vacuum and in the presence or absence of air, or an inert gas. For example, in an embodiment, one or more steps are done under vacuum, in the absence of air under an inert gas.

In one or more embodiments, likewise packaging in canisters may be done with or without vacuum and in the presence or absence of air, or an inert gas.

### Example 2—Example of a Clinical Study in Patients with Epidermal-Growth-Factor-Receptor (EGFR) Inhibitor-Associated Skin Toxicity

### 1. Study Synopsis

STUDY TITLE: A randomized, double blind, placebo controlled, in a multi-center Phase II clinical trial, to assess the safety and tolerability of topically applied FDX104 antibiotic foam and to obtain preliminary evidence on efficacy of FDX104 in the prevention and reduction of Epidermal Growth Factor Receptor Inhibition (hereinafter “EGFRI”) skin toxicity (hereinafter “a pulopustular rash” or “EGFRI associated rash” or “EGFRI induced rash” “EGFRI rash”), in subjects with cancer receiving cetuximab or panitumumab.

OBJECTIVES: (i) To assess the safety and tolerability of topical FDX104 antibiotic foam in a population of cancer patients receiving EGFRI treatment twice daily, in the morning and in the evening, adjunct to either Cetuximab or Panitumumab treatment; (ii) To detect preliminary efficacy of FDX104 antibiotic foam for prevention and reduction of EGFRI toxicity.

STUDY MEDICATION: Doxycycline Hyclate foam (4% compositions, as described in section 9 below) and placebo (vehicle foam).

DOSAGE: Patients are treated topically on facial skin areas affected by EGFRI toxicity twice daily in the morning and evening for 5 weeks. One side of the face of a subject is treated with doxycycline foam while the other side of the face is treated with placebo (vehicle).

INDICATION: EGFRI skin toxicity.

DESIGN: A Phase II, randomized, double (Investigator, patient) blind, placebo controlled, multi center, clinical trial, to assess the safety, tolerability and efficacy of topically applied doxycycline foam in the prevention or reduction of EGFRI rash in subjects with advanced cancer treated by EGFRI.

The study consists of a pre-treatment period: Screening, informed consent, eligibility criteria, safety laboratory examinations, treatment period where patients are randomized and begin topical treatment on the facial skin areas affected by EGFRI rash twice daily for 5 weeks, followed by a post-treatment follow up visit 4 weeks after end of treatment. Seven days (+3 days) after randomization and study drug initiation, subjects start their EGFRI treatment. During the study drug treatment period, all subjects attend periodic study-center visits: 3 weeks (+3 days) after study drug initiation, and 5 weeks after study drug initiation in order to assess safety and efficacy of the treatment.

Evaluations consist mainly of: Rash severity grading by using the MESTT and the Visual Scale of Rash Severity (Scope A.) method, and safety assessments. These evaluations are performed on visit 2 (week 3) and visit 4 (week 5).

During the follow-up period which is conducted at visit 5 (week 9), 4 weeks from the completion of visit 4, only subjects who are experiencing possibly-related or related adverse events at visit 4, which have not resolved to baseline level, are assessed for safety.

Excluded from safety assessments are those subjects that ran out of study by the Escape criteria, (see below) and received one of the drugs listed in the exclusion criteria as their results could be confounded by new non study treatments given to them by the investigator at his discretion, or by their treating oncologist. Treatment after completion of the study is at the discretion of the investigator. With the investigator's consent, subjects, who express desire to continue treatment of one type of the study products, may continue to do so on a blinded basis.

Further treatment is allowed only in the absence of unresolved possibly-related or related adverse events at visit 4 and if there are additional canisters of the assigned treatment at the pharmacy by the end of the study treatment. If patient and investigator decide to continue treatment, the investigator chooses one of the treatments on a blinded basis (Left (L) or Right (R) labeled). The patient is instructed to use the chosen treatment for his/her entire face at the same regimen used throughout the study and as described in the protocol, until these canisters are used.

Escape criteria: Study Treatment is terminated before end of treatment visit (visit 4) and subject enters into the non-treatment follow-up period but is allowed to start treatment for the rash as per clinicians instruction if: (a) the rash reaches grade 3 (MESTT scale) on one side of the face and this grade exceeds the grade of rash on the other side of the face by two grade levels; or (b) the rash reaches grade 3 (MESTT scale) on one side of the face and this grade exceeds the grade of rash on the other side of the face by one grade level for two consecutive weekly visits; or (c) the rash reaches grade 3 (MESTT scale) on both sides of the face for at least one week, or (d) the rash reaches grade 3 (MESTT scale) and/or severe pain or severe pruritus occurs on any part of his/her body due to the rash associated with EGFRIs. If weekly visits are needed to confirm an escape criterion, an unscheduled visit should be performed.

VARIABLES: Efficacy, safety and tolerability in the treatment of EGFRI rash.

PATIENTS: 24 patients (male and female patients), age 18 years and older, scheduled to start Cetuximab or Panitumumab treatment; subjects must not be on EGFRI treatment prior to randomization. If prior cycles of the EGFRI-treatment regimen were administered to the subject, this treatment must have been terminated 3-months prior to randomization. Females must be non-pregnant, postmenopausal or undertaking contraceptive measures.

### 2. Clinical Study Design

The protocol and informed consent forms are approved by each clinical site's local Ethics Committee (EC) and the Israel Ministry of Health prior to study initiation. To be eligible for the study, the subjects are required to sign a written informed consent document and are willing and able to comply with the requirements of the protocol. Subjects over the age of 18 are enrolled and randomized on a blinded basis (Left (L) or Right (R) labeled) to be applied on either side of the face of a subject, testing efficacy, safety and tolerability of doxycycline 4% versus placebo between the two facial sides.

Treatment is administered topically on facial skin areas affected with EGFRI rash twice daily, morning and evening, for 5 weeks. The mode of application is demonstrated by the investigator or study nurse at Visit 0 using a placebo from a demonstration kit that is supplied by the Applicants. Subjects are instructed to cleanse their face with a mild or soapless, non-medicated cleanser and then pat it dry. They are instructed to shake the canister before use, dispense a small amount of foam on one finger and then apply a small amount of the foam using the tip of their finger for one side of the face: cheeks and chin. Subjects were instructed to treat the nose area only if affected and to apply the foam on the whole area, not just on visible lesions. Application is attained by collapsing and spreading it as a thin layer on the affected area. Patients are further instructed not to apply moisturizers, new brands of make-up, creams, lotions, powders or any topical product other than the assigned treatment to the treatment area. Patients are instructed to minimize exposure to sunlight, including sunlamps, while using the compositions. Use of sunscreen products over treated areas is recommended when sun exposure cannot be avoided.

TABLE 1
study assessment table

| | <14 | | | | | Day | |
|---|---|---|---|---|---|---|---|
| | Days of | Day 1 | Day | Day | Day | 35 ± 3 | |
| | random | Treatment | 14 ± 3 | 21 ± 3 | 28 ± 3 | End of | Day |
| | Screening | Initiation | Treatment | Treatment | Treatment | Treatment | 63 ± 5 |
| | Visit | Visit 0 | Visit 1 | Visit 2 | Visit 3 | Visit 4 | Visit 5/FU |
| Obtain Informed Consent | X | | | | | | |
| Eligibility Criteria | X | | | | | | |
| β-subunit hCG blood | X | | | | | | |
| pregnancy test | | | | | | | |
| Urine pregnancy test | | | | X | | X | |
| Medical History/Surgical History | X | | | | | | |
| Subject Demographics | X | | | | | | |
| Race and Ethnicity | X | | | | | | |
| Vital Signs (BP, HR, temp, RR) | X | X | | X | | X | |
| Weight and Height (w/o shoes) | X | | | | | | |
| Blood and urine laboratory tests | X | | | | | | |
| EGFRI start date documentation | | X | | | | | |
| Method of Birth Control | X | | | | | | |
| MESTT scale | | X | | X | | X | |
| Digital Photograph of Face | | X | | X | | X | |
| Fitzpatrick Skin Phototype | X | | | | | | |
| Randomization for Side of Face | | X | | | | | |
| Dispense Study | | X | | X | | | |
| Weigh Study Medications | | X | | X | | X | |
| and Record | | | | | | | |
| Concomitant Medications | X | X | | X | | X | X |
| Adverse Events | | X | | X | | X | X |
| Collect Study Medications | | | | X | | X | |

Compliance of patients is estimated by weighing each container before and after use, and calculating the amount of medication used by each patient and by examining the patient diaries which include recordings of daily drug applications.

Safety assessments consist of evaluating skin tolerability, adverse events, serious adverse events and vital signs. In those cases of an existing difference in rash severity (using the MESTT scale) between the two facial sides, whereby the more severe grade is observed on the FDX104 treated side, laboratory examinations, performance status and documentation of all concomitant medications and/or therapies are included in the safety evaluation.

Efficacy

Efficacy parameters are assessed by the following methods: (a) MESTT scale, (b) the visual scale of rash severity—Scope photographs method and (c) the Fitzpatrick photo-type classification.

MESTT scale—The eruption rate and grade is assessed according to the MESTT scale defined in appendix 1. The MESTT scale is performed by the local investigator according to the visit schedule (see FIG. 1).

Scope photographs method—The eruption rate and grade is assessed according to: The visual scale of rash severity—Scope A; defined and visualized in appendix 2: The Scope scale is used by a blinded trained clinician in the field, reviewing the digital photographs of the subjects according to the following classification: none, mild, moderate, and severe.

Fitzpatrick photo-type classification—(see appendix 3) is performed at the screening visit and serve for sub-analysis of the efficacy based on skin type.

### 3. Patient Demographics

Safety Analysis Population (SAF)

All randomized subjects, who receive at least one dose of the study drug.

Intent-to-Treat Efficacy Population (ITT)

All randomized subjects, who receive at least one dose of the study drug.

(The SAF and the ITT populations for this study are the same).

An all-completers analysis is to be performed. A sensitivity analysis using multiple imputation with longitudinal modeling may be used for patients who fail to complete all follow-up measurements.

Per Protocol Analysis Population (PP)

Per-protocol set (PPS): All SAF subjects with no major protocol deviations who complete the treatment phase as planned are to be analyzed according to the original assigned treatment group.

### 4. Statistical Methodology

Data from all subjects who receive any study drug is to be included in the safety analyses. The severity of the toxicities to be graded according to the Common Terminology Criteria for Adverse Events (CTCAE) (version 4.0), formerly called the Common Toxicity Criteria (CTC or NCI-CTC). In the by-subject analysis, a subject having the same event more than once will be counted only once. Adverse events are to be summarized by the worst CTCAE grade. Adverse events leading to death or to discontinuation from treatment, events classified as CTCAE Grade III or Grade IV, study-drug-related events, and serious adverse events are to be summarized separately. Laboratory data will be graded according to CTCAE severity grade.

The safety and the tolerability analysis are to be done for the safety population. AEs are to be coded by the CTCAE, SOC (System Organ Class), preferred term and grade. Incidences of AEs are to be categorized according to: seriousness, severity (grade), relationship to study drug, action taken and outcome of event. For the summary by severity, subjects who have multiple occurrences of the same AE are classified according to the worst reported severity of the AE. In the by-subject analysis, a subject having the same event more than once are counted only once. For the summary by relationship to study drug, subjects who have multiple occurrences of the same AE are classified according to the strongest reported relationship to study medication. The AE variables are categorical variables.

For categorical variables, numbers and percentages are to be calculated. For continuous variable, ranges, medians, means and standard deviations are to be calculated. Distributions for categorical variables are to be compared and analyzed by the Chi square test (a parametric test) or by Fisher-Irwin exact test (a non-parametric test for small sample). Test for normality will be done by Shapiro-Wilk normality test. The results between pairs of continuous variables are to be analyzed by paired t-test (a parametric test) or by Wilcoxon paired signed-rank test (a non-parametric test for small numbers). All statistical tests are to be analyzed to a significance level of 0.05.

### 5. Clinical Response to Treatment

The clinical response to treatment, (clinical success or clinical failure) is derived from an efficacy evaluation using the following methods and scales:

Efficacy is evaluated by the following parameters:
1. Overall efficacy, mean maximal rash grade on treatment side vs. vehicle side
2. Incidences of serious rash (grades 2-3) on treatment side vs. vehicle side
3. Sub-analysis of the mean maximal rash grade on treatment side vs. vehicle side based on skin photo-type classification (Fitzpatrick, 1.988).

Safety and tolerability is evaluated by vital signs and the incidence and severity of AEs.
1. Mean maximal rash grade on treatment side vs. vehicle side
2. No. of incidences with maximal skin rash (grade 1-3)
3. No. of incidences with maximal skin rash grade 1
4. No. of incidences with maximal skin rash grade 2
5. No. of incidences with maximal skin rash grade 3
6. No. of incidences with maximal skin rash (grade 2-3).
The above is evaluated once by the investigator using the MESTT Papulopustular eruption grading scale and once by a blinded trained clinician in the field who will review all digital photographs and classify the rash severity using Scope photographs method (Scope A 2009).
7. Sub-analysis of the efficacy (first exploratory end-point) based on skin photo-type classification (Fitzpatrick, 1988).

### Escape Criteria (for Success and Failure):

Success

Rash reaches grade 3 (MESTT scale) on one side of the face and this grade exceeds the other side by two grade levels for one week; or

Rash reaches grade 3 (MESTT scale) on one side of the face and this grade exceeds the grade on the other side by one grade levels for two weeks

Failure:

Rash reaches grade 3 (MESTT scale) and/or severe pain or severe pruritus occurs on both sides of the face for one week or any other part of his/her body due to the rash associated with EGFRIs MESTT scale

### 6. Safety Tolerability and Adverse Events

Safety and tolerability is determined for all randomized patients by the investigator at each visit. Safety is assessed using different parameters such as vital signs (blood pressure, heart rate, temperature), physical examination of body systems, pregnancy potential. Tolerability (clinical assessment of skin irritation) is determined by evaluation of adverse events such as, erythema, dryness, pigmentation, peeling and itching at each study visit and at follow up. Based on patient subjective assessment, itching is evaluated.

New concomitant medications are recorded since the previous visit and/or changes in previously recorded continuing concomitant medications since the previous visit as well adverse events (AE's) since the previous visit and/or changes in AE's, which had continued since the previous visit. All adverse experiences are classified by the investigator as either unrelated; unlikely related; suspected or probably related to the study drug.

In one or more embodiments similar clinical studies can be conducted for any tetracycline formulations described herein, such as DOX331, DOX332, DOD-003, MCD-037, MCD-045, MCD-052 MCD-053 and MCD-058.

### 7. Compositions

The below compositions, for use in the clinical study, are prepared according to the manufacturing procedures detailed in Example 1.

TABLE 2A
Formulations 1% Minocycline and 4% Minocycline

Formulations

| | 244B | 244A |
|---|---|---|
| | (1% Minocycline) | (4% Minocycline) |
| Ingredients | % w/w | % w/w |
| Light Mineral oil | 4.44 | 1.11 |
| Cyclomethicone | 5.00 | 5.00 |
| Coconut oil | 23.60 | 23.60 |
| Soybean oil | 50.00 | 50.00 |
| Hydrogenated castor oil | 2.00 | 2.00 |
| Beeswax | 2.00 | 2.00 |
| Myristyl alcohol | 2.50 | 2.50 |
| Cetostearyl alcohol | 3.50 | 3.50 |
| Stearyl alcohol | 1.50 | 1.50 |
| Behenyl alcohol | 1.10 | 1.10 |
| Fumed Silica (SiO2) | 0.25 | 0.25 |
| Stearic acid | 3.00 | 3.00 |
| Minocycline HCl | 1.11 | 4.44 |
| (micronized) | | |
| (90% potency) | | |
| Total | 100 | 100 |
| Propellant AP-70 | 12.00 | 12.00 |

TABLE 2B
Formulations 1% Minocycline and 4% Minocycline without silica

| | 4% FOAM | 1% FOAM |
|---|---|---|
| | Quantitative | Quantitative |
| | Composition | Composition |
| Component | (% w/w) | (% w/w) |
| Minocycline | 4.00^{a} | 1.00^{a} |
| Hydrochloride | | |
| (micronized) (expressed | | |
| as minocycline) | | |
| Soybean Oil | 50.00 | 50.00 |
| Coconut Oil | 23.60 | 23.60 |
| Light Mineral Oil | 0.91-1.37^{b} | 4.58-4.69^{b} |
| Cyclomethicone | 5.00 | 5.00 |
| Cetostearyl Alcohol | 3.50 | 3.50 |
| Stearic Acid | 3.00 | 3.00 |
| Myristyl Alcohol | 2.50 | 2.50 |
| Hydrogenated Castor Oil | 2.00 | 2.00 |
| White Wax (Beeswax) | 2.00 | 2.00 |
| Stearyl Alcohol | 1.50 | 1.50 |
| Docosanol | 1.10 | 1.10 |
| Total Bulk | 100 | 100 |
| AP-70 (butane + | 12.0 | 12.0 |
| isobutane + propane)^{C} | | |

^{a}The amount of minocycline hydrochloride is adjusted by the potency of the minocycline hydrochloride.
^{b}The amount of light mineral oil in the formulation is adjusted based on the amoud of minocycline hydrochloride.
^{C}AP-70 (CAS # 6847-86-8) is a mixture of about 27% w/w butane, 18% w/w isobutene and 55% w/w propane.

TABLE 2C
Formulation of DOX-244B

| Ingredient Name | % W/W |
|---|---|
| Coconut oil | 23.60 |
| Mineral oil light | 4.35 |
| soybean oil | 50.00 |
| stearic acid | 3.00 |
| behenyl alcohol | 1.10 |
| hydrogenated castor oil | 2.00 |
| Beeswax | 2.00 |
| Stearyl alcohol | 1.50 |
| Cetostearyl alcohol | 3.50 |
| Myristyl alcohol | 2.50 |
| Cyclomethicone | 5.00 |
| Silicon dioxide | 0.25 |
| Doxycycline Hyclate (micronized) | 1.20 |

TABLE 2D
Formulation of FDX104 and placebo

| | FDX-104 | |
|---|---|---|
| | 4% FOAM | |
| | Quantitative | FDX-104 Placebo FOAM |
| | Composition | Quantitative Composition |
| Component | (% w/w) | (% w/w) |
| Doxycycline hyclate | 4.00^{a} | — |
| (micronized) (expressed | | |
| as doxycycline) | | |
| Soybean Oil | 50.00 | 50.00 |
| Coconut Oil | 23.60 | 23.60 |
| Light Mineral Oil | 0.95-1.21^{b} | 5.80 |
| Cyclomethicone | 5.00 | 5.00 |
| Cetostearyl Alcohol | 3.50 | 3.50 |
| Stearic Acid | 3.00 | 3.00 |
| Myristyl Alcohol | 2.50 | 2.50 |
| Hydrogenated Castor Oil | 2.00 | 2.00 |
| White Wax (Beeswax) | 2.00 | 2.00 |
| Stearyl Alcohol | 1.50 | 1.50 |
| Docosanol | 1.10 | 1.10 |
| Total Bulk | 100 | 100 |
| AP-70 (butane + | 12.0 | 12.0 |
| isobutane + propane)^{C} | | |

^{a}The amount of doxycycline hyclate is adjusted by the potency of the doxycycline hyclate.
^{b}The amount of light mineral oil in the formulation is adjusted based on the amout of doxycycline hyclate.
^{C}AP-70 (CAS # 6847-86-8) is a mixture of about 27% w/w butane, 18% w/w isobutene and 55% w/w propane.

TABLE 2E
Formulations of DOX331 and DOX332

Formulations

| | DOX331 | DOX332 |
|---|---|---|
| Ingredient | % w/w | % w/w |
| Mineral oil, heavy* | 82.24 | 88.24 |
| Mineral oil, light | 5.00 | — |
| Stearyl alcohol | 4.50 | 3.70 |
| Steam acid | 2.50 | 2.50 |
| Behenyl alcohol | 1.10 | 0.70 |
| Paraffin 51-53 | — | 0.20 |
| doxycycline hyclate | 4.66 | 4.66 |
| (micronized)** | | |
| Total | 100.00 | 100.00 |
| AP-70 | 12% | 12% |

*The amount of heavy mineral oil in the formulation is adjusted based on the amound of doxycycline hyclate.
**The amount of doxycycline hyclate is adjusted by the potency of the doxycycline hyclate.

TABLE 2F
Formulation of Doxycycline and adapalene

| | Formulations |
|---|---|
| | DOD-003 |
| Ingredient | % w/w |
| Mineral oil heavy* | 81.94 |
| Mineral oil light | 5 |
| Stearyl alcohol | 4.5 |
| Stearic acid | 2.5 |
| Behenyl alcohol | 1.1 |
| Doxycycline | 4.66 |
| hyclate | |
| (micronized)** | |
| Adapalene | 0.3 |
| Total | 100 |
| AP-70 | 12% |

*The amount of heavy mineral oil in the formulation is adjusted based on the amount of doxycycline hyclate.
**The amount of doxycycline hyclate is adjusted by the potency of the doxycycline hyclate.

TABLE 2G
Formulations of Minocycline and adapalene

| | MCD- | MCD- | MCD- | MCD- | MCD- |
|---|---|---|---|---|---|
| Component | 037 | 045 | 052 | 053 | 058 |
| Mineral oil “heavy”* | 82.00 | 88.60 | | 49.00 | 43.40 |
| Mineral oil light* | 5.00 | | 0.70 | 39.00 | 39.00 |
| Myristyl alcohol | | | 2.50 | | |
| Cetostearyl alcohol | | | 3.50 | | |
| Stearyl alcohol | 4.50 | 3.60 | 1.50 | 3.80 | 4.30 |
| Stearic acid | 2.50 | 2.40 | 3.00 | 2.40 | 2.50 |
| Cyclomethicone 5 | | | 5.00 | | 5.00 |
| Coconut oil | | | 23.60 | | |
| Soybean oil | | | 50.00 | | |
| Behenyl alcohol | 1.10 | 0.50 | 1.0 | 0.70 | 0.70 |
| Beeswax | | | 2.00 | | |
| Hydrogenated castor oil | | | 2.00 | | |
| MCH (micronized)** | 4.80 | 4.80 | 4.80 | 4.80 | 4.80 |
| Adapalene | 0.10 | 0.10 | 0.30 | 0.30 | 0.30 |
| Total | 100.00 | 100.00 | 100.00 | 100.00 | 100.00 |
| AP-70 | 12% | 12% | 12% | 12% | 12% |

*The amount of heavy mineral oil or light mineral oil in the formulation is adjusted based on the amount of Minocycline hydrochloride.
**The amount of minocycline hydrochloride is adjusted by the potency of the minocycline hydrochloride.

All inactive ingredients used in the formulation are intended for topical use and listed in the current FDA Inactive Ingredient Database; concentrations used do not exceed the maximum concentrations given in Database.

### Example 3—Chemical and Physical Stability

The achievement of a long term stable foamable formulation of tetracycline antibiotics described herein, was a major challenge and required both extensive research and creativity.

The chemical and physical stability results of minocycline HCl (MCH) and doxycycline hyclate (“DOX”) in oleaginous formulations, MCH244 and DOX244, respectively, are described in U.S. application Ser. No. 14/147,376 (U.S. Pub. No. 2014/0121188) and incorporated by reference herein. In an accelerated stability study, samples were stored at 40° C., and the concentrations of minocycline HCl and doxycycline hyclate were determined by UPLC. Stability test for MCH244 results following 2 months, 3 months, 6 months, 9 months, 12 months, 18 months, and 24 months of storage are shown herein below.

The following examples illustrate the chemical stability of minocycline HCl (“FDX”) and doxycycline hyclate (“FDX104”) in oleaginous formulations, as described in Tables 4, 5, and 7-9 below. In an accelerated stability study, samples were stored at 40° C., and the concentrations of minocycline HCl and doxycycline hyclate were determined by UPLC. The stability test results following 2 months, 3 months, 6 months, 9 months, 12 months, and 18 months of storage are shown herein below.

Samples of MCH244 and DOX244 1% and 4% were stored at 25° C. and 40° C. in order to test physical and chemical stability.

### 1. Inspection of Formulation in Glass Bottles

The use of pressurized glass bottles enables the inspection of formulations for homogeneity in the presence of propellant. Following 18 months of storage at 25° C. the formulation was found to be re-dispersible, i.e., homogeneous following slight shaking.

### 2. Stability Following Storage at 25° C. and 40° C.

Storage at 25° C. and 40° C. for 18 months revealed almost no change in the Minocycline concentration. Test results for chemical stability of minocycline following storage for up to 18 months at 25° C. and 40° C. are summarized in Table 3 and Table 4. There was practically no degradation of 244 1% and 4% minocycline following 18 months at 25° C. and also following 9 months at 40° C. These stability results indicate shelf life of more than two years at ambient temperature. Test results for chemical stability of doxycycline following storage for up to 9 months at 25° C. and 40° C. are summarized in Tables 5-7. There was practically no degradation of doxycycline following 6 months at 25° C. and at 40° C. These stability results likewise indicate a long shelf life of more than two years at ambient temperature. In one or more embodiments, the tetracycline composition has a shelf life of at least 6 months, or at least 9 months, or at least 12 months, or at least 15 months, or at least 18 months, or at least 21 months, or at least 24 months at ambient temperature. In one or more embodiments, the tetracycline composition has a shelf life of at least 6 months, or at least 9 months, or at least 12 months, or at least 15 months, or at least 18 months, or at least 21 months, or at least 24 months at 25° C. In one or more embodiments, the tetracycline composition has a shelf life of at least 1 month, or at least 3 months, or at least 6 months, or at least 9 months, or at least 12 months at 40° C.

TABLE 3
Minocycline content in MCH244 1% following storage
for 18 months at 25° C. and 40° C.

Minocycline content (% w/w)

| Temp | T = 0 | 3 M | 6 M | 9 M | 12 M | 18 M |
|---|---|---|---|---|---|---|
| 25° C. | 1.001 | NM | 0.986 | 1.007 | 0.972 | 0.959 |
| 40° C. | 1.001 | 1.002 | 0.983 | 0.965 | NM | NM |

NM = not measured

TABLE 4
Minocycline content in FDX104 4% following storage for 18 months
at 25° C. and 40° C. (Lot MCH-244-100825)

Minocycline content (% w/w)

| Temp | T = 0 | 3 M | 6 M | 9 M | 12 M | 18 M |
|---|---|---|---|---|---|---|
| 25° C. | 1.012 | NM | 0.998 | 0.998 | 0.972 | 0.925 |
| 40° C. | 1.012 | 0.963 | 1.009 | 0.978 | NM | NM |

### Minocycline Physical Stability:

The results for physical stability following storage at 25° C. and 40° C. for 18 months were as follows:

Foam quality: Conformed to the foam quality specification following 9 months storage at 40° C.

Odor: Conformed to the specifications and showed no odor following storage at 40° C. for 9 months.

Color: The color of the formulation remained light, slightly changed to grey-yellow following storage at 40° C. for 9 months. No change was observed at 25° C.

Shakability: Conformed to specifications following storage at 40° C. for 9 months.

Density: No significant change in density was found after storage at 40° C. for 9 months.

Collapse time: No change in foam collapse time (the time for the foam to reach half of its initial height) was found in any of the formulation samples tested after storage for 9 months at 40° C.

Microscopic observations: No significant change in the microscopic appearance was noted following storage at 40° C. for 9 months.

Corrosion and deterioration: The coated aluminum surfaces of the can and valve and the plastic housing of the valve appeared fully intact and showed no signs of corrosion or deterioration. No changes in color or deformation were observed.

### Doxycycline DOX-244B Physical and Chemical Stability:

The results for physical stability following storage at 25° C. for 18 months and for 24 months were as follows:

Foam quality: excellent.

Collapse time: At least 180 seconds.

Production: GMP Compliance.

For the purpose of clinical supplies, the production of the compositions was performed according to the principles of current good manufacturing practice (c-GMP). Production conditions were aimed to ensure high quality of the product and to prevent any potential cross contamination. The production site was certified by the Israel Ministry of Health as suitable for GMP production and supply of small clinical batches for Phase I and IIa clinical trials.

The below composition was prepared according to the manufacturing procedures detailed in Example 1.

TABLE 5
Formulation of DOX-244B-111123

| Ingredient Name | % W/W |
|---|---|
| Coconut oil | 23.60 |
| Mineral oil light | 4.35 |
| soybean oil | 50.00 |
| stearic acid | 3.00 |
| behenyl alcohol | 1.10 |
| hydrogenated castor oil | 2.00 |
| Beeswax | 2.00 |
| Stearyl alcohol | 1.50 |
| Cetostearyl alcohol | 3.50 |
| Myristyl alcohol | 2.50 |
| Cyclomethicone | 5.00 |
| Silicon dioxide | 0.25 |
| Doxycycline Hyclate (micronized) | 1.20 |

TABLE 6
Doxycycline % content in DOX-244B-111123 PF following storage for 9 months
at 5° C., 25° C., 40° C., and 50° C.

| | Doxycycline content (% w/w) | | | | | | | |
|---|---|---|---|---|---|---|---|---|
| Batch/Sample | | 1 M | | | 2 M | | 3 M | |
| name | T = 0 | 5° C. | 25° C. | 50° C. | 25° C. | 50° C. | 25° C. | 40° C. |
| DOX-244- | 1.0220 | 1.031 | 1.022 | — | — | — | 1.010 | 1.031 |
| 111123 PF | | | | | | | | |
| DOX-244- | 1.0800 | 1.098 | 1.080 | 1.060 | — | 1.045 | 1.082 | 1.046 |
| 111123 PFF | | | | | | | | |

| | Doxycycline content (% w/w) | | | | | |
|---|---|---|---|---|---|---|
| Batch/Sample | 6 M | | 9 M | 12 M | 18 M | 24 M |
| name | 25° C. | 40° C. | 25° C. | 25° C. | 25° C. | 25° C. |
| DOX-244- | 1.017 | 1.025 | 1.053 | 0.967 | 0.994 | 1.021 |
| 111123 PF | | | | | | |
| DOX-244- | 1.046 | 1.028 | 1.091 | 1.044 | 1.018 | 1.051 |
| 111123 PFF | | | | | | |

TABLE 7
Stability of Doxycycline Foam at 25° C. and 40° C.
%¹ Doxycycline Hyclate in DOX244 foam product

| Months | 40° C. (foam) | 25° C. (foam) |
|---|---|---|
| 0 | 102.2 | 102.2 |
| 1 | | 102.2 |
| 2 | | |
| 3 | 103.1 | 101.0 |
| 6 | 102.5 | 101.7 |
| 9 | | 105.3 |
| 12 | | 96.7 |
| 18 | | 99.4 |
| 24 | | 102.1 |

¹The percentage are derived from the PF figures in Table 6. Note 1.2% doxycycline hyclate is equivalent to 1.0176%. doxycycline based on USP

TABLE 8
Degradation of Doxycycline at 5° C., 25° C., 40° C., and 50° C.

| Degradation | Batch/Sample | DOX-244B-111123 | DOX-244B-111123 |
|---|---|---|---|
| product w/w | name | PF | PFF |
| T0 | RRT 0.75 | 0.003 | 0.004 |
| | RRT 0.85 | 0.010 | 0.011 |
| 1 M | 5° C. | 0.003 | 0.003 |
| | RRT 0.75 | | |
| | 5° C. | 0.010 | 0.010 |
| | RRT 0.85 | | |
| | 25° C. | 0.003 | 0.003 |
| | RRT 0.75 | | |
| | 25° C. | 0.010 | 0.010 |
| | RRT 0.85 | | |
| | 50° C. | — | 0.003 |
| | RRT 0.75 | | |
| | 50° C. | — | 0.01 |
| | RRT 0.85 | | |
| 2 M | 50° C. | — | 0.003 |
| | RRT 0.75 | | |
| | 50° C. | — | 0.009 |
| | RRT 0.85 | | |
| 3 M | 25° C. | 0.003 | 0.004 |
| | RRT 0.75 | | |
| | 25° C. | 0.01 | 0.01 |
| | RRT 0.85 | | |
| | 40° C. | 0.003 | 0.003 |
| | RRT 0.75 | | |
| | 40° C. | 0.01 | 0.01 |
| | RRT 0.85 | | |
| 6 M | 25° C. | 0.003 | 0.003 |
| | RRT 0.75 | | |
| | 25° C. | 0.01 | 0.01 |
| | RRT 0.85 | | |
| | 40° C. | 0.003 | 0.003 |
| | RRT 0.75 | | |
| | 40° C. | 0.01 | 0.01 |
| | RRT 0.85 | | |
| 9 M | 25° C. | 0.003 | 0.003 |
| | RRT 0.75 | | |
| | 25° C. | 0.009 | 0.01 |
| | RRT 0.85 | | |
| 12 M | 25° C. | 0.003 | 0.003 |
| | RRT 0.75 | | |
| | 25° C. | 0.009 | 0.009 |
| | RRT 0.85 | | |
| 18 M | 25° C. | 0.003 | 0.003 |
| | RRT 0.75 | | |
| | 25° C. | 0.009 | 0.009 |
| | RRT 0.85 | | |
| 24 M | 25° C. | 0.003 | 0.003 |
| | RRT 0.75 | | |
| | 25° C. | 0.009 | 0.009 |
| | RRT 0.85 | | |

TABLE 9
Appearance and Collapse time of Doxycycline at 25° C. and 40° C.

| | Appearance | Appearance | Collapse time (25° C.) | | Collapse time (40° C.) | |
|---|---|---|---|---|---|---|
| Time | (25° C.) | (40° C.) | Collapse | Time to | Collapse | Time to |
| Points | Quality | Quality | time(sec) | FG(sec) | time(sec) | FG(sec) |
| T0 | G | — | 100 | 150 | — | — |
| 3 M | E | G | 115 | 90 | 165 | 90 |
| 6 M | E | G | >180 | 120 | >180 | >180 |
| 9 M | E | — | 150 | 120 | — | — |
| 12 M | G | — | 105 | 120 | — | — |
| 18 M | E | — | >180 | >180 | — | — |
| 24 M | E | — | >180 | >180 | — | — |

### Doxycycline DOX-330A-140331 Physical and Chemical Stability:

The results for physical stability following storage at 25° C. for 9 months and for 12 months were as follows:

Foam quality: Excellent,

Collapse time: At least 180 seconds.

Production: GMP Compliance.

For the purpose of clinical supplies, the production of the compositions was performed according to the principles of current good manufacturing practice (c-GMP). Production conditions were aimed to ensure high quality of the product and to prevent any potential cross contamination. The production site was certified by the Israel Ministry of Health as suitable for GMP production and supply of small clinical batches for Phase I and IIa clinical trials.

The below composition was prepared according to the manufacturing procedures detailed in Example 1.

TABLE 10
Formulation of DOX-330A-140331

| Ingredient Name | % W/W |
|---|---|
| Coconut oil | 23.60 |
| Mineral oil light | 0.90 |
| soybean oil | 50.00 |
| stearic acid | 3.00 |
| behenyl alcohol | 1.10 |
| hydrogenated castor oil | 2.00 |
| Beeswax | 2.00 |
| Stearyl alcohol | 1.50 |
| Cetostearyl alcohol | 3.50 |
| Myristyl alcohol | 2.50 |
| Cyclomethicone | 5.00 |
| Doxycycline Hyclate (micronized) | 4.90 |

TABLE 11
Doxycycline % content in DOX-330A-140331 PF following storage
for 12 months at 25° C. and 40° C.

| | | | | | | | |
|---|---|---|---|---|---|---|---|
| Batch/Sample | T = 0 | 3 w | 2 M | 3 M | 6 M | 9 M | 12 M |

| name | | 40° C. | 40° C. | 25° C. | 40° C. | 25° C. | 40° C. | 25° C. | 25° C. |
|---|---|---|---|---|---|---|---|---|---|
| DOX-330A- | 4.032 | 3.984 | 3.981 | 4.086 | 3.942 | 4.086 | 4.088 | 3.993 | 4.032 |
| 140331 PF | | | | | | | | | |

TABLE 12
Stability of Doxycycline Foam at 25° C. and 40° C.
%² Doxycycline Hyclate DOX330 foam product

| Months | 40° C. (foam) | 25° C. (foam) |
|---|---|---|
| 0 | 100.8 | 100.8 |
| 0.75 | 99.6 | |
| 2 | 99.5 | |
| 3 | 98.6 | 102.2 |
| 6 | 102.2 | 102.2 |
| 9 | | 99.8 |
| 12 | | 100.8 |
| 18 | | |
| 24 | | |

²The percentages are derived from the PF figures in Table 11. Note 1.2% doxycycline hyclate is equivalent to 1.0176%. doxycycline based on USP

TABLE 13
Degradation of Doxycycline at 25° C. and 40° C.

| Degradation | Batch/Sample | |
|---|---|---|
| product w/w | name | DOX-330A-140331 PF |
| T0 | RRT 0.85 | — |
| 3 w | 40° C. | 0.017 |
| | RRT 0.85 | |
| 2 M | 40° C. | 0.014 |
| | RRT 0.85 | |
| 3 M | 25° C. | 0.016 |
| | RRT 0.85 | |
| | 40° C. | 0.016 |
| | RRT 0.85 | |
| 6 M | 25° C. | 0.017 |
| | RRT 0.85 | |
| | 40° C. | 0.017 |
| | RRT 0.85 | |
| 9 M | 25° C. | 0.020 |
| | 6-epi (RRT 0.85) | |
| 12 M | 25° C. | 0.0213 |
| | 6-epi (RRT 0.85) | |

TABLE 14
Appearance and Collapse time of Doxycycline at 25° C. and 40° C.

| | Appearance | Appearance | Collapse time (25° C.) | | Collapse time (40° C.) | |
|---|---|---|---|---|---|---|
| Time | (25° C.) | (40° C.) | Collapse | Time to | Collapse | Time to |
| Points | Quality | Quality | time(sec) | FG(sec) | time(sec) | FG(sec) |
| T0 | E | — | 160 | 180 | — | — |
| 3 W | — | E- falls a | — | — | >180 | >180 |
| | | little | | | | |
| 2 M | — | E | — | — | >180 | >180 |
| 3 M | E | E | >180 | >180 | 180 | >180 |
| 6 M | E | E | >180 | >180 | >180 | >180 |
| 9 M | E | — | >180 | >180 | — | — |
| 12 M | E | — | >180 | >180 | — | — |

### Doxycycline Physical and Chemical Stability:

The results for physical stability following storage at 25° C. for 9 months and for 6 months were as follows:

Foam quality: Excellent.

Odor: No odor

Collapse time: At least 120 seconds.

Production: GMP Compliance.

For the purpose of clinical supplies, the production of the compositions was performed according to the principles of current good manufacturing practice (c-GMP). Production conditions were aimed to ensure high quality of the product and to prevent any potential cross contamination. The production site was certified by the Israel Ministry of Health as suitable for GMP production and supply of small clinical batches for Phase I and IIa clinical trials.

The below composition was prepared according to the manufacturing procedures detailed in Example 1.

TABLE 15
Formulation of DOX-330A-140818 PF (FDX104)

| Ingredient Name | % W/W |
|---|---|
| Coconut oil | 23.60 |
| Mineral oil light | 1.13 |
| soybean oil | 50.00 |
| stearic acid | 3.00 |
| behenyl alcohol | 1.10 |
| hydrogenated castor oil | 2.00 |
| Beeswax | 2.00 |
| Stearyl alcohol | 1.50 |
| Cetostearyl alcohol | 3.50 |
| Myristyl alcohol | 2.50 |
| Cyclomethicone | 5.00 |
| Doxycycline Hyclate (micronized) | 4.67 |

TABLE 16
Doxycycline % content in DOX-330A-140818 (FDX104) PF following
storage for 9 months at 25° C. and 40° C.

| | Doxycycline content (% w/w) | | | | | | | | | |
|---|---|---|---|---|---|---|---|---|---|---|
| Batch/Sample | | 1 M | 3 M | | 6 M | | 9 M | 12 M | 18 M | 24 M |
| name | T = 0 | 40° C. | 25° C. | 40° C. | 25° C. | 40° C. | 25° C. | 25° C. | 25° C. | 25° C. |

| | | | | | | | |
|---|---|---|---|---|---|---|---|
| DOX-330A- | 3.908 | 3.899 | 3.792 | 3.763 | 3.727 | 3.783 | 3.763 |
| 140818 PF | | | | | | | |

TABLE 17
Stability of Doxycycline Foam at 25° C. and 40° C.
%³ Doxycycline in DOX330 140818

| Months | 40° C. (foam) | 25° C. (foam) |
|---|---|---|
| 0 | 97.7 | 97.7 |
| 1 | 97.5 | |
| 3 | 94.1 | 94.8 |
| 6 | 94.6 | 93.2 |
| 9 | | 94.1 |
| 12 | | |
| 18 | | |
| 2.4 | | |

³The percentages are derived from the PF figures in Table 16. Note 1.2% doxycycline hyclate is equivalent to 1.0176%, doxycycline based on USP

TABLE 18
Degradation of Doxycycline at 25° C. and 40° C.

| Degradation | | |
|---|---|---|
| product w/w | Batch/Sample name | DOX330 140818 |
| T = 0 | RRT 0.85 | 0.016 |
| | (6-epi) | |
| 1 M | 40° C. | 0.017 |
| | RRT 0.85 (6-epi) | |
| 3 M | 25° C. | 0.019 |
| | 6-epi | |
| | 40° C. | 0.019 |
| | 6-epi | |
| 6 M | 25° C. | 0.019 |
| | 6-epi | |
| | 40° C. | 0.019 |
| | 6-epi | |
| 9 M | 25° C. | 0.019 |
| | 6-epi | |

TABLE 19
Appearance and Collapse time of Doxycycline at 25° C. and 40° C.

| | Appearance | Appearance | Collapse time (25° C.) | | Collapse time (40° C.) | |
|---|---|---|---|---|---|---|
| Time | (25° C.) | (40° C.) | Collapse | Time to | Collapse | Time to |
| Points | Quality | Quality | time(sec) | FG(sec) | time(sec) | FG(sec) |
| T0 | E | — | 155 | 180 | | |
| 1 M | E | — | 90 | >180 | | |
| 3 M | E | E | 180 | >180 | 150 | >180 |
| 6 M | E | E | 180 | >180 | >180 | >180 |
| 9 M | E | — | 120 | >180 | | |

### Doxycycline and Adapalene DOD-003 Physical and Chemical Stability:

TABLE 20
Doxycycline % content in DOD-003 following storage
for 1 month at 25° C., 40° C. and 60° C.

| | Doxycycline content (% w/w) | | | | |
|---|---|---|---|---|---|
| Batch/Sample | T = 0 | | 1 M | | |
| name | 25° C./40° C. | 60° C. | 25° C. | 40° C. | 60° C. |
| DOD-003 | 3.850 | 3.880 | 3.949 | 3.860 | 3.824 |

TABLE 21
Stability of Doxycycline at 25° C., 40° C. and 60° C.
%⁴ Doxycycline in DOD-003

| Months | 25° C. (foam) | 40° C. (foam) | 60° C. (Pre foam formulation) |
|---|---|---|---|
| 0 | 96.3 | 96.3 | 97.0 |
| 1 | 98.7 | 96.5 | 95.6 |

⁴The percentages are derived from the figures in Table 20.

TABLE 22
Degradation of Doxycycline at 25° C., 40° C. and 60° C.

| Degradation | | |
|---|---|---|
| product w/w | Batch/Sample name | DOD-003 |
| T = 0 | 25° C. 6-epi | 0.021 |
| | 40° C. 6-epi | 0.021 |
| | 60° C. 6-epi | 0.022 |
| 1 M | 25° C. 6-epi | 0.022 |
| | 40° C. 6-epi | 0.021 |
| | 60° C. 6-epi | 0.021 |

TABLE 23
Appearance, Collapse time and shakability of Doxycycline at 25° C. and 40° C. in DOD-003

| | | | | Collapse time | | | | | |
|---|---|---|---|---|---|---|---|---|---|
| | Appearance | | | 25° C. | | 40° C. | | 60° C. | |
| Time | 25° C. | 40° C. | 60° C. | Collapse | Time to | Collapse | Time to | Collapse | Time to |
| Points | Quality | Quality | Qualify | time (s) | FG (s) | time (s) | FG (s) | time (s) | FG (s) |
| T0 | E | E | NM | >180 | >180 | >180 | >180 | NM | NM |
| 1 M | E | E | NM | >180 | 120 | >180 | 120 | NM | NM |

| Time Points | Shakability (25° C.) | Shakability (40° C.) | Shakability (60° C.) |
|---|---|---|---|
| T0 | 2 | 2 | NM |
| 1 M | 0 | 2 | NM |

TABLE 24
Adapalene % content in DOD-003 following storage
for 1 month at 25° C., 40° C. and 60° C.

| | Adapalene content (% w/w) | | | | |
|---|---|---|---|---|---|
| Batch/Sample | T = 0 | | 1 M | | |
| name | 25° C./40° C. | 60° C. | 25° C. | 40° C. | 60° C. |
| DOD-003 | 0.2948 | 0.2948 | 0.3030 | 0.2950 | 0.3076 |

TABLE 25
Stability of Adapalene at 25° C., 40° C. and 60° C.
%⁵ Adapalen in DOD-003

| Months | 25° C. (foam) | 40° C. (foam) | 60° C. (Pre foam formulation) |
|---|---|---|---|
| 0 | 98.3 | 98.3 | 98.3 |
| 1 | 101.0 | 98.3 | 102.5 |

⁵The percentages are derived from the figures in Table 24.

### Minocycline and Adapalene MCD-037-160320 Physical and Chemical Stability:

TABLE 26
Minocycline % content in MCD-037-160320 following storage for 4 months at 25° C. and 40° C.

| | Minocycline content (% w/w) | | | | | | | | |
|---|---|---|---|---|---|---|---|---|---|
| Batch/Sample | T = 0 | 1 M | | 2 M | | 3 M | | 4 M | |
| name | 25° C./40° C. | 25° C. | 40° C. | 25° C. | 40° C. | 25° C. | 40° C. | 25° C. | 40° C. |
| MCD-037-160320 | 3.89 | NM | 3.90 | NM | 4.03 | 3.88 | 3.89 | 3.99 | 3.95 |

TABLE 27
Stability of Minocycline at 25° C. and 40° C.
%⁶ Minocycline in MCD-037-160320

| Months | 25° C. (foam) | 40° C. (foam) |
|---|---|---|
| 0 | 97.22 | 97.22 |
| 1 | NM | 97.62 |
| 2 | NM | 100.68 |
| 3 | 97.01 | 97.30 |
| 4 | 99.66 | 98.79 |

⁶The percentages are derived from the figures in Table 26.

TABLE 28
Degradation of Minocycline at 25° C. and 40° C.

| Degradation | | |
|---|---|---|
| product w/w | Batch/Sample name | MCD-037-160320 |
| T = 0 | 25° C. 4-epi | 0.06 |
| | 40° C. 4-epi | 0.06 |
| 1 M | 25° C. 4-epi | NM |
| | 40° C. 4-epi | 0.10 |
| 2 M | 25° C. 4-epi | NM |
| | 40° C. 4-epi | 0.08 |
| 3 M | 25° C. 4-epi | 0.07 |
| | 40° C. 4-epi | 0.09 |
| 4 M | 25° C. 4-epi | 0.09 |
| | 40° C. 4-epi | 0.10 |

TABLE 29
Appearance, Collapse time, shakability and homogeneity of Minocycline
at 25° C. and 40° C. in MCD-037-160320

| | Appearance | Appearance | Collapse time (25° C.) | | Collapse time (40° C.) | |
|---|---|---|---|---|---|---|
| Time | (25° C.) | (40° C.) | Collapse | Time to | Collapse | Time to |
| Points | Quality | Quality | time(sec) | FG(sec) | time(sec) | FG(sec) |
| T0 | E | E | >180 | >180 | >180 | >180 |
| 1 M | NM | E | NM | NM | >180 | >180 |
| 2 M | NM | E | NM | NM | >180 | >180 |
| 3 M | E | E | >180 | >180 | >180 | >180 |
| 4 M | E | E | >180 | >180 | >180 | >180 |

| Time | Shakability | Shakability | | |
|---|---|---|---|---|
| Points | (25° C.) | (40° C.) | Homogeneity (25° C.) | Homogeneity (40° C.) |
| T0 | 2 | 2 | Crystals uniformly | Crystals uniformly |
| | | | disdtributed | disdtributed |
| 1 M | NM | 2 | NM | Crystals uniformly |
| | | | | disdtributed |
| 2 M | NM | 2 | NM | Crystals uniformly |
| | | | | disdtributed |
| 3 M | 2 | 2 | Crystals uniformly | Crystals uniformly |
| | | | disdtributed | disdtributed |
| 4 M | 2 | 2 | Crystals uniformly | Crystals uniformly |
| | | | disdtributed | disdtributed |

TABLE 30
Adapalene % content in MCD-037-160320 following storage for 4 months at 25° C. and 40° C.

| | Adapalene content (% w/w) | | | | | | | | |
|---|---|---|---|---|---|---|---|---|---|
| | T = 0 | | | | | | | | |
| Batch/Sample | 25° C./ | 1 M | | 2 M | | 3 M | | 4 M | |
| name | 40° C. | 25° C. | 40° C. | 25° C. | 40° C. | 25° C. | 40° C. | 25° C. | 40° C. |
| MCD-037-160320 | 0.09 | NM | 0.10 | NM | 0.10 | 0.10 | 0.10 | 0.0967 | 0.10 |

TABLE 31
Stability of Adapalene at 25° C. and 40° C.
%⁷ Adapalene in MCD-037-160320

| Months | 25° C. (foam) | 40° C. (foam) |
|---|---|---|
| 0 | 93.9 | 93.9 |
| 1 | NM | 96.9 |
| 2 | NM | 95.7 |
| 3 | 96.4 | 97.4 |
| 4 | 96.7 | 97.10 |

⁷The percentages are derived from the figures in Table 30.

### Minocycline and Adapalene MCD-045-160306 Physical and Chemical Stability:

TABLE 32
Minocycline % content in MCD-045-160306 following storage
for 3 months at 25° C. and 40° C.

| | Minocycline content (% w/w) | | | | | | |
|---|---|---|---|---|---|---|---|
| | T = 0 | | | | | | |
| Batch/Sample | 25° C./ | 1 M | | 2 M | | 3 M | |
| name | 40° C. | 25° C. | 40° C. | 25° C. | 40° C. | 25° C. | 40° C. |
| MCD-045-160306 | 3.83 | NM | 4.21 | NM | 3.93 | 3.91 | 3.96 |

TABLE 33
Stability of Minocycline at 25° C. and 40° C.
%⁸ Minocycline in MCD-045-160306

| Months | 25° C. (foam) | 40° C. (foam) |
|---|---|---|
| 0 | 95.76 | 95.76 |
| 1 | NM | 105.15 |
| 2 | NM | 98.14 |
| 3 | 97.79 | 99.08 |

⁸The percentages are derived from the figures in Table 32.

TABLE 34
Degradation of Minocycline at 25° C. and 40° C.

| Degradation | | |
|---|---|---|
| product w/w | Batch/Sample name | MCD-045-160306 |
| T = 0 | 25° C. 4-epi | 0.07 |
| | 40° C. 4-epi | 0.07 |
| 1 M | 25° C. 4-epi | NM |
| | 40° C. 4-epi | 0.11 |
| 2 M | 25° C. 4-epi | NM |
| | 40° C. 4-epi | 0.07 |
| 3 M | 25° C. 4-epi | 0.08 |
| | 40° C. 4-epi | 0.10 |

TABLE 35
Appearance, Collapse time, shakability and homogeneity of Minocycline
at 25° C. and 40° C. in MCD-045-160306

| | Appearance | Appearance | Collapse time (25° C.) | | Collapse time (40° C.) | |
|---|---|---|---|---|---|---|
| Time | (25° C.) | (40° C.) | Collapse | Time to | Collapse | Time to |
| Points | Quality | Quality | time(sec) | FG(sec) | time(sec) | FG(sec) |
| T0 | E | E | >180 | >180 | >180 | >180 |
| 1 M | NM | E | NM | NM | >180 | >180 |
| 2 M | NM | E | NM | NM | >180 | >180 |
| 3 M | E | E | >180 | >180 | >180 | 150 |
| 4 M | E | E- | >180 | >180 | 140 | 120 |

| Time | Shakability | Shakability | | |
|---|---|---|---|---|
| Points | (25° C.) | (40° C.) | Homogeneity (25° C.) | Homogeneity (40° C.) |
| T0 | 2 | 2 | Crystals uniformly | Crystals uniformly |
| | | | disdtributed | disdtributed |
| 1 M | NM | 2 | NM | Crystals uniformly |
| | | | | disdtributed |
| 2 M | NM | 2 | NM | Crystals uniformly |
| | | | | disdtributed |
| 3 M | 2 | 2 | Crystals uniformly | Crystals uniformly |
| | | | disdtributed | disdtributed |
| 4 M | 2 | 2 | Crystals uniformly | Crystals uniformly |
| | | | disdtributed | disdtributed |

TABLE 36
Adapalene % content in MCD-045-160306 following storage
for 3 months at 25° C. and 40° C.

| | Adapalene content (% w/w) | | | | | | |
|---|---|---|---|---|---|---|---|
| Batch/Sample | T = 0 | 1 M | | 2 M | | 3 M | |
| name | 25° C./40° C. | 25° C. | 40° C. | 25° C. | 40° C. | 25° C. | 40° C. |
| MCD-045-160306 | 0.10 | NM | 0.10 | NM | 0.10 | 0.09 | 0.10 |

TABLE 37
Stability of Adapalene at 25° C. and 40° C.
%⁹ Adapalene in MCD-045-160306

| Months | 25° C. (foam) | 40° C. (foam) |
|---|---|---|
| 0 | 95.10 | 95.03 |
| 1 | NM | 101.13 |
| 2 | NM | 95.80 |
| 3 | 94.40 | 95.83 |

⁹The percentages are derived from the figures in Table 36.

### Minocycline and Adapalene MCD-052-160410 Physical and Chemical Stability:

TABLE 38
Minocycline % content in MCD-052-160410 following storage
for 3 months at 25° C. and 40° C.

| | Minocycline content (% w/w) | | | | | | |
|---|---|---|---|---|---|---|---|
| Batch/Sample | T = 0 | 1 M | | 2 M | | 3 M | |
| name | 25° C./40° C. | 25° C. | 40° C. | 25° C. | 40° C. | 25° C. | 40° C. |
| MCD-052-160410 | 3.94 | 3.88 | 3.71 | 3.82 | 3.80 | 3.81 | 3.84 |

TABLE 39
Stability of Minocycline at 25° C. and 40° C.
%¹⁰ Minocycline in MCD-052-160410

| Months | 25° C. (foam) | 40° C. (foam) |
|---|---|---|
| 0 | 98.48 | 98.48 |
| 1 | 97.02 | 92.75 |
| 2 | 95.43 | 95.08 |
| 3 | 95.30 | 96.05 |

¹⁰The percentages are derived from the figures in Table 38.

TABLE 40
Degradation of Minocycline at 25° C. and 40° C.

| Degradation | | |
|---|---|---|
| product w/w | Batch/Sample name | MCD-052-160410 |
| T = 0 | 25° C. 4-epi | 0.09 |
| | 40° C. 4-epi | 0.09 |
| 1 M | 25° C. 4-epi | 0.07 |
| | 40° C. 4-epi | 0.06 |
| 2 M | 25° C. 4-epi | 0.08 |
| | 40° C. 4-epi | 0.08 |
| 3 M | 25° C. 4-epi | 0.09 |
| | 40° C. 4-epi | 0.07 |

TABLE 41
Appearance, Collapse time, shakability and homogeneity of Minocycline
at 25° C. and 40° C. in MCD-052-160410

| | Appearance | Appearance | Collapse time (25° C.) | | Collapse time (40° C.) | |
|---|---|---|---|---|---|---|
| Time | (25° C.) | (40° C.) | Collapse | Time to | Collapse | Time to |
| Points | Quality | Quality | time(sec) | FG(sec) | time(sec) | FG(sec) |
| T0 | E | E | >180 | >180 | >180 | >180 |
| 1 M | E | E | >180 | >180 | >180 | >180 |
| 2 M | E | E | >180 | >180 | >180 | >180 |
| 3 M | E | E | NM | NM | NM | NM |

| Time | Shakability | Shakability | | |
|---|---|---|---|---|
| Points | (25° C.) | (40° C.) | Homogeneity (25° C.) | Homogeneity (40° C.) |
| T0 | 2 | 2 | Crystals uniformly | Crystals uniformly |
| | | | disdtributed | disdtributed |
| 1 M | 2 | 2 | Crystals uniformly | Crystals uniformly |
| | | | disdtributed | disdtributed |
| 2 M | 2 | 2 | Crystals uniformly | Crystals uniformly |
| | | | disdtributed | disdtributed |
| 3 M | 0 | 0 | Crystals uniformly | Crystals uniformly |
| | | | disdtributed | disdtributed |

TABLE 42
Adapalene % content in MCD-052-160410 following storage
for 3 months at 25° C. and 40° C.

| | Adapalene content (% w/w) | | | | | | |
|---|---|---|---|---|---|---|---|
| Batch/Sample | T = 0 | 1 M | | 2 M | | 3 M | |
| name | 25° C./40° C. | 25° C. | 40° C. | 25° C. | 40° C. | 25° C. | 40° C. |
| MCD-052-160410 | 0.29 | 0.29 | 0.29 | 0.30 | 0.30 | 0.30 | 0.29 |

TABLE 43
Stability of Adapalene at 25° C. and 40° C.
%¹¹ Adapalene in MCD-052-160410

| Months | 25° C. (foam) | 40° C. (foam) |
|---|---|---|
| 0 | 97.31 | 97.31 |
| 1 | 95.50 | 96.74 |
| 2 | 98.69 | 99.00 |
| 3 | 99.16 | 97.78 |

¹¹The percentages are derived from the figures in Table 42.

### Minocycline and Adapalene MCD-053-160413 Physical and Chemical Stability:

TABLE 44
Minocycline % content in MCD-053-160413 following storage
for 3 months at 25° C. and 40° C.

| | Minocycline content (% w/w) | | | | | | |
|---|---|---|---|---|---|---|---|
| Batch/Sample | T = 0 | 1 M | | 2 M | | 3 M | |
| name | 25° C./40° C. | 25° C. | 40° C. | 25° C. | 40° C. | 25° C. | 40° C. |
| MCD-053-160413 | 3.91 | 3.95 | 3.91 | 3.84 | 3.88 | 3.97 | 3.94 |

TABLE 45
Stability of Minocycline at 25° C. and 40° C.
%¹² Minocycline in MCD-053-160413

| Months | 25° C. (foam) | 40° C. (foam) |
|---|---|---|
| 0 | 97.79 | 97.79 |
| 1 | 98.64 | 97.69 |
| 2 | 96.03 | 96.98 |
| 3 | 99.32 | 98.43 |

¹²The percentages are derived from the figures in Table 44.

TABLE 46
Degradation of Minocycline at 25° C. and 40° C.

| Degradation | | |
|---|---|---|
| product w/w | Batch/Sample name | MCD-053-160413 |
| T = 0 | 25° C. 4-epi | 0.07 |
| | 40° C. 4-epi | 0.07 |
| 1 M | 25° C. 4-epi | 0.07 |
| | 40° C. 4-epi | 0.08 |
| 2 M | 25° C. 4-epi | 0.08 |
| | 40° C. 4-epi | 0.09 |
| 3 M | 25° C. 4-epi | 0.11 |
| | 40° C. 4-epi | 0.11 |

TABLE 47
Appearance, Collapse time, shakability and homogeneity of Minocycline
at 25° C. and 40° C. in MCD-053-160413

| | Appearance | Appearance | Collapse time (25° C.) | | Collapse time (40° C.) | |
|---|---|---|---|---|---|---|
| Time | (25° C.) | (40° C.) | Collapse | Time to | Collapse | Time to |
| Points | Quality | Quality | time(sec) | FG(sec) | time(sec) | FG(sec) |
| T0 | E | E | >180 | >180 | >180 | >180 |
| 1 M | E | E | >180 | 60 | >180 | >180 |
| 2 M | E | E | 175 | 180 | 180 | 180 |

| Time | Shakability | Shakability | | |
|---|---|---|---|---|
| Points | (25° C.) | (40° C.) | Homogeneity (25° C.) | Homogeneity (40° C.) |
| T0 | 2 | 2 | Crystals uniformly | Crystals uniformly |
| | | | disdtributed | disdtributed |
| 1 M | 2 | 2 | Crystals uniformly | Crystals uniformly |
| | | | disdtributed | disdtributed |
| 2 M | 2 | 2 | Crystals uniformly | Crystals uniformly |
| | | | disdtributed | disdtributed |

TABLE 48
Adapalene % content in MCD-053-160413 following storage
for 3 months at 25° C. and 40° C.

| | Adapalene content (% w/w) | | | | | | |
|---|---|---|---|---|---|---|---|
| Batch/Sample | T = 0 | 1 M | | 2 M | | 3 M | |
| name | 25° C./40° C. | 25° C. | 40° C. | 25° C. | 40° C. | 25° C. | 40° C. |
| MCD-053-160413 | 0.28 | 0.28 | 0.28 | 0.28 | 0.28 | 0.29 | 0.28 |

TABLE 49
Stability of Adapalene at 25° C. and 40° C.
%¹³ Adapalene in MCD-053-160413

| Months | 25° C. (foam) | 40° C. (foam) |
|---|---|---|
| 0 | 93.41 | 93.41 |
| 1 | 94.66 | 93.42 |
| 2 | 91.80 | 93.78 |
| 3 | 95.44 | 94.07 |

¹³The percentages are derived from the figures in Table 48.

### Minocycline and Adapalene MCD-058-160414 Physical and Chemical Stability:

TABLE 50
Minocycline % content in MCD-058-160414 following storage
for 3 months at 25° C. and 40° C.

| | Minocycline content (% w/w) | | | | | | |
|---|---|---|---|---|---|---|---|
| Batch/Sample | T = 0 | 1 M | | 2 M | | 3 M | |
| name | 25° C./40° C. | 25° C. | 40° C. | 25° C. | 40° C. | 25° C. | 40° C. |
| MCD-058-160414 | 4.06 | 3.90 | 3.97 | 3.92 | 4.01 | 3.91 | 3.90 |

TABLE 51
Stability of Minocycline at 25° C. and 40° C.
%¹⁴ Minocycline in MCD-058-160414

| Months | 25° C. (foam) | 40° C. (foam) |
|---|---|---|
| 0 | 101.61 | 101.61 |
| 1 | 97.58 | 99.20 |
| 2 | 97.88 | 100.36 |
| 3 | 97.76 | 97.50 |

¹⁴The percentages are derived from the figures in Table 50.

TABLE 52
Degradation of Minocycline at 25° C. and 40° C.

| Degradation | | |
|---|---|---|
| product w/w | Batch/Sample name | MCD-058-160414 |
| T = 0 | 25° C. 4-epi | 0.09 |
| | 40° C. 4-epi | 0.09 |
| 1 M | 25° C. 4-epi | 0.09 |
| | 40° C. 4-epi | 0.10 |
| 2 M | 25° C. 4-epi | 0.09 |
| | 40° C. 4-epi | 0.10 |
| 3 M | 25° C. 4-epi | 0.12 |
| | 40° C. 4-epi | 0.12 |

TABLE 53
Appearance, Collapse time, shakability and homogeneity of Minocycline
at 25° C. and 40° C. in MCD-058-160414

| | Appearance | Appearance | Collapse time (25° C.) | | Collapse time (40° C.) | |
|---|---|---|---|---|---|---|
| Time | (25° C.) | (40° C.) | Collapse | Time to | Collapse | Time to |
| Points | Quality | Quality | time(sec) | FG(sec) | time(sec) | FG(sec) |
| T0 | E | E | >180 | 150 | >180 | 150 |
| 1 M | E | E | >180 | >180 | >180 | >180 |
| 2 M | E | E | >180 | 180 | >180 | 180 |

| Time | Shakability | Shakability | | |
|---|---|---|---|---|
| Points | (25° C.) | (40° C.) | Homogeneity (25° C.) | Homogeneity (40° C.) |
| T0 | 2 | 2 | Crystals uniformly | Crystals uniformly |
| | | | disdtributed | disdtributed |
| 1 M | 2 | 2 | Crystals uniformly | Crystals uniformly |
| | | | disdtributed | disdtributed |
| 2 M | 2 | 2 | Crystals uniformly | Crystals uniformly |
| | | | disdtributed | disdtributed |

TABLE 54
Adapalene % content in MCD-058-160414 following storage
for 3 months at 25° C. and 40° C.

| | Adapalene content (% w/w) | | | | | | |
|---|---|---|---|---|---|---|---|
| Batch/Sample | T = 0 | 1M | | 2M | | 3M | |
| name | 25° C./40° C. | 25° C. | 40° C. | 25° C. | 40° C. | 25° C. | 40° C. |
| MCD-058-160414 | 0.29 | 0.27 | 0.28 | 0.28 | 0.29 | 0.29 | 0.29 |

TABLE 55
Stability of Adapalene at 25° C. and 40° C.
%¹⁵ Adapalene in MCD-058-160414

| Months | 25° C. (foam) | 40° C. (foam) |
|---|---|---|
| 0 | 95.01 | 95.01 |
| 1 | 91.47 | 94.05 |
| 2 | 93.64 | 95.11 |
| 3 | 95.87 | 95.09 |

¹⁵The percentages are derived from the figures in Table 54.

### Doxycycline DOX331 Physical and Chemical Stability:

TABLE 56
Doxycycline % content in DOX331 following
storage for 1 week at 25° C. and 40° C.

| | Doxcycline content (% w/w) | |
|---|---|---|
| Batch/Sample | T = 0 | 1 Week |

| name | 25° C. | 40° C. | 25° C. | 40° C. |
|---|---|---|---|---|
| DOX331 | 4.146 | 4.146 | 4.103 | 4.106 |

TABLE 57
Stability of Doxycycline at 25° C. and 40° C.
%¹⁶ Doxycycline in DOX331

| Time | 25° C. | 40° C. |
|---|---|---|
| 0 | 103.7 | 103.7 |
| 1 week | 102.6 | 102.7 |

¹⁶The percentages are derived from the figures in Table 56.

TABLE 58
Degradation of Doxycycline at 25° C. and 40° C.

| Degradation | | |
|---|---|---|
| product w/w | Batch/Sample name | DOX331 |
| T = 0 | 25° C. 6-epi | 0.026 |
| | 40° C. 6-epi | 0.026 |
| 1 Week | 25° C. 6-epi | 0.026 |
| | 40° C. 6-epi | 0.025 |

TABLE 59
Appearance, Collapse time and shakability of
Doxycycline at 25° C. and 40° C. in DOX331

| | Appearance | Appearance | | |
|---|---|---|---|---|
| | (25° C.) | (40° C.) | Shakability | Shakability |
| Time Points | Quality | Quality | (25° C.) | (40° C.) |
| T0 | E | E | 0 | 0 |
| 1 week | E | E | 1 | 1 |

### Doxycycline DOX332 Physical and Chemical Stability:

TABLE 60
Doxycycline % content in DOX332 following storage for 1 week
at 25° C. and 40° C.

| | Doxycycline content (% w/w) | |
|---|---|---|
| Batch/Sample | T = 0 | 1 Week |

| name | 25° C. | 40° C. | 25° C. | 40° C. |
|---|---|---|---|---|
| DOX332 | 4.074 | 4.074 | 4.124 | 4.169 |

TABLE 61
Stability of Doxycycline at 25° C. and 40° C.
%¹⁷ Doxycycline in DOX332

| Time | 25° C. | 40° C. |
|---|---|---|
| 0 | 101.8 | 101.8 |
| 1 week | 103.1 | 104.2 |

¹⁷The percentages are derived from the figures in Table 60.

TABLE 62
Degradation of Doxycycline at 25° C. and 40° C.

| Degradation | | |
|---|---|---|
| product w/w | Batch/Sample name | DOX332 |
| T = 0 | 25° C. 6-epi | 0.026 |
| | 40° C. 6-epi | 0.026 |
| 1 Week | 25° C. 6-epi | 0.026 |
| | 40° C. 6-epi | 0.026 |

TABLE 63
Appearance, Collapse time and shakability of Doxycycline
at 25° C. and 40° C. in DOX332

| | Appearance | Appearance | | |
|---|---|---|---|---|
| | (25° C.) | (40° C.) | Shakability | Shakability |
| Time Points | Quality | Quality | (25° C.) | (40° C.) |
| T0 | E | E | 1 | 1 |
| 1 week | E | E | 1 | 1 |

### Example 4—Clinical Study Phase I (4% Minocycline Foam) PK Study Under Maximum Use Conditions for 16 Days

Study Synopsis

Cancer patients receiving cancer therapy are faced with a high burden on their internal organs and blood circulating system. Additional drugs given systemically will increase this burden and in many cases could result in exhaustion and failure of patients' internal organs. A method for ameliorating anti-tumor treatment-induced skin toxicity comprising administrating a topical tetracycline composition will allow delivery of the drug directly to the target organ while avoiding side effects to internal organs and minimizing the systemic burden.

In this example, topical administration of tetracycline (for example minocycline) was studied and the pharmacokinetic profile of the drug and its bioavailability was characterized.

STUDY TITLE: An Open-label, Multiple Dose Study to Assess the Pharmacokinetic Profile of Minocycline from FMX-101 Foam (4%) in Male and Female Volunteers.

OBJECTIVES: 1. To assess bioavailability of minocycline from FMX-101 minocycline HCl foam, 4%. 2. To characterize the pharmacokinetic profile of minocycline following multiple-dose topical administration of FMX-101 (4%) in healthy volunteers with or without acne.

STUDY MEDICATION: FMX-101 minocycline (4%)—approximately 4 gr per application.

DOSAGE FORM: Foam.

INDICATION: Acne vulgaris.

DESIGN: An open-label, single-center, non-randomized, multiple administrations study in males and females, some of which are with acne. Twelve (12) subjects (at least 4 subjects with acne) enrolled to receive a daily dose of topical FMX-101 minocycline (4%) foam for sixteen consecutive days.

Eligible subjects were admitted to the Clinical Research Center (CRC) in the evening before the first study drug administration (Day 0), and remained in-house for 24 hours after first dosing (Day 1). Throughout this day blood samples for PK were drawn at time points specified below. After receiving the second dose (Day 2) they were released from the CRC.

Subjects then arrived at the CRC on the mornings of Days 3, 5, 7, 8, 9, 10, 11, 12, 14 and 15. They remained under supervision in the CRC, with the application areas uncovered, for 30 min before being released. On Days 3, 7, 9, 11 and 14, blood was drawn for PK (trough) within 10 min before the subjects received the study drug.

On days 4, 6 and 13 the drug was applied at home by the subject according to the Investigator/study staff instructions.

On the evening of Day 15 the subjects were re-admitted to the CRC. On Day 16 they received the last (sixteenth) dose and went through the same procedures as in Day 1. After being released form the CRC they were required to attend three additional ambulatory PK blood sampling (36, 48 and 60 hours post-dose).

PK Evaluation Timing of PK Blood Sampling

Blood samples to determine plasma of minocycline were collected at the following time points:
Day 1: pre-dose (within 90 min before first dosing), 30 min. 1, 2, 4, 8, 12, 16 hours post-dose and Day 2 at 24 hrs post-dose within 10 min before second dosing—a total of 9 samples).
Days 3, 7, 9, 11 and 14: pre-dose (trough) samples, within 10 min before drug application.
Day 16: pre-dose (within 10 min before drug application), 30 min, 1, 2, 4, 8, 12, 16 hours post-dose, Day 17, 24 (±10 min) hours post-dose (before discharge from the CRC) and additional ambulatory PKs at 36 (±15 min), (Day 17), 48 (±30 min) and 60 (±30 min) hours after last drug application (Day 18)—a total of 12 blood samples.

TABLE 64
PK sampling scheme

| Study Day | Time relative to dosing | |
|---|---|---|
| Day 1 | 0 | h (Predose) |
| | 0.5 | h |
| | 1 | h |
| | 2 | h |
| | 4 | h |
| | 8 | h |
| | 12 | h |
| | 16 | h |
| Day 2 | Predose (24 h postdose) | |
| Day 3 | Predose (24 h postdose) | |
| Day 7 | Predose (24 h postdose) | |
| Day 9 | Predose (24 h postdose) | |
| Day 11 | Predose (24 h postdose) | |
| Day 14 | Predose (24 h postdose) | |
| Day 16 | 0 | h (Predose, 24 h postdose) |

| | |
|---|---|
| | 0.5 |
| | 1 |
| | 2 |
| | 4 |
| | 8 |
| | 12 |
| | 16 |
| Day 17 | 24 |
| | 36 |
| Day 18 | 48 |
| | 60 |

Throughout a period of 18 days a total of 26 samples per subject were drawn for PK.

Calculation of Pharmacokinetic Parameters

PK of minocycline was derived from plasma concentration versus time data. For purposes of calculating PK parameters, concentrations <LLQ were treated as zero. For purposes of tabular presentation and graphing mean profiles, concentration values <LLQ were treated as missing.

The PK parameters assessed included:

Cmax—Maximum plasma concentration achieved (dosing days 1 and 16).

AUCT—The area under the plasma concentration versus time curve in ng*mL/h. The AUC from time zero to the last experimental time point (t*) with a detectable drug concentration equal to or greater than the limit of quantification value were designated AUCT and calculated by the linear trapezoidal rule (dosing days 1 and 16).

All calculated concentration values were electronically transferred. Individual subject PK parameter values were derived by non-compartmental methods by WinNonlin 6.3 within the Phoenix 64 software package. The peak plasma concentration (Cmax) was obtained from experimental observations.

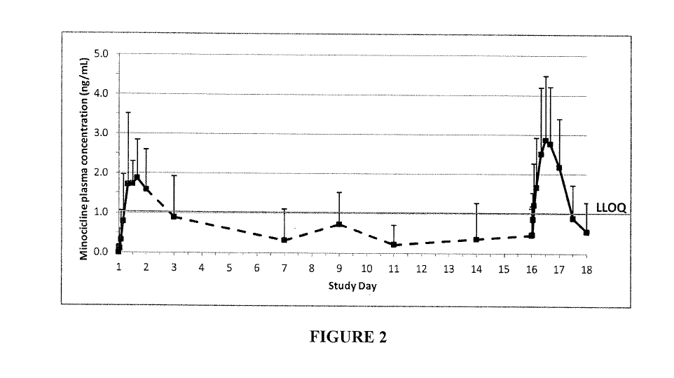

FIG. 2 depicts the mean minocycline plasma concentrations from Day 1 to Day 16 for subjects who received FMX-101.

Results:

TABLE 65
FMX101-1 PK parameters
PK Non-Compartmental Analysis Summary Statistics (Day 1, Day 16)

| Parameter | Day 1 | Day 16 |
|---|---|---|
| Cₘₐₓ [ng/mL] | 2.26 ± 1.60 | 5.04 ± 6.19 |
| AUC_{T} [ng · h/mL] | 33.83 ± 22.73 | 84.36 ± 48.36 |

Analysis:

In general, the observed minocycline plasma concentrations throughout the study were low and close to the sensitive lower limit of quantification (LLOQ=1.1 ng/mL).

The results of this study showed a very low absorption, with the Cmax (Day 16)=5 ng/mL, about 500 times lower than the Cmax and AUC for the labeled dose of the oral extended release minocycline, Solodyn® (100-135 mg).

Similar PK studies for additional tetracycline antibiotics, such as doxycycline in one or more embodiments may be undertaken. For example, PK studies for doxycycline in formulations such as FDX104, DOX331, DOX332, DOD-003, MCD-037, MCD-045, MCD-052 MCD-053 and MCD-058.

Doxycycline is generally regarded as non-toxic in short term treatment, as indicated by its oral acute toxicity of LD50=1007 mg/kg (mouse).

Chronic toxicity of doxycycline was evaluated in rats at oral doses up to 500 mg/kg/day for 18 months. Findings revealed no adverse effects on growth, food consumption, or survival.

Oral doxycycline administration may cause common side effects, including upset stomach, nausea, diarrhea and mild headache.

Since doxycycline hyclate is a larger molecule compared to minocycline HCl, in some embodiments it can have a reduced penetration and hence the maximum plasma concentrations can be less than those obtained for minocycline HCl.

### Example 5—Compatibility Study

Procedure: Minocycline hydrochloride (“MCH”) was incubated as a suspension with various excipients at 25° C. and 40° C. for maximum of sixty days or to the point where degradation was suspected. The ratio between MCH and the tested excipient is detailed below. Visual inspection was the major criterion for indication of compatibility. The color of intact MCH suspension is pale yellow; and any change of color (e.g., to dark orange, red, green, brown and black) indicates oxidation or degradation.

Hydrophilic solvents were tested for compatibility with MCH at a ratio of MCH: excipient of 1:250. Dimethyl Isosorbide, Glycerin, Ethanol, Propylene glycol, Butylene Glycol, PEG 200, Hexylene Glycol, PEG 400, Dimethyl Sulfoxide and Diethylene glycol monoethyl ether were found to be incompatible with MCH.

Oily emollients and waxes were tested for compatibility with MCH at a ratio of MCH:excipient of 1:250 for Oily emollients and 1:50 for waxes. Hydrogenated castor oil, Castor oil, Cocoglycerides, Disopropyl adipate, Mineral oil light, Coconut oil, Beeswax, MCT oil, Cyclomethicone, Isododecane, Cetearyl octanoate, Gelled mineral oil, Isopropyl myristate, PPG 15 stearyl ether, Mineral oil heavy, Octyl dodecanol, White Petrolatum, Petrolatum (Sofmetic), Paraffin 51-53, Paraffin 51-53. Paraffin 58-62, Calendula oil, Shea butter, Grape seed oil, Almond oil, Jojoba oil, Avocado oil, Peanut oil, Wheat germ oil and Hard Fat were found to be compatible with MCH. Pomegranate seed oil was found to be incompatible with MCH. Other than hydrogenated castor oil, beeswax, paraffin and hard fat, the aforesaid items listed are oily emollients.

The compatibility of MCH with hydrophobic surfactant was tested following solubilization of the surfactant in mineral oil (mineral oil was previously shown to be compatible with MCH). Surfactants were tested for compatibility with MCH at a ratio of MCH:excipient of 1:50. PEG 150 distearate, Laureth 4, PEG 40 hydrogenated castor oil, PEG 75 lanolin, Glucam P20 distearate, PEG 100 stearate, Glyceryl monostearate, PEG 40 stearate, Montanov S (Cocoyl Alcohol (and) C12-20 Alkyl Glucoside), Alkyl lactate. Benton gel, SPAN 60, Sorbitan sesquistearate, SPAN 40, SPAN 80, Tween 20, Ceteth 2, Sucrose stearic acid esters D1813, Ceteareth 20, Steareth 2/Steareth 21, Methyl glucose sesquistearate, Oleth 20, PPG 20 methyl glucose ether, Tween 60 were found to be incompatible with MCH. Sucrose stearic acid esters D1803, Sucrose stearic acid esters D1807 and Sucrose stearic acid esters D1811 were found to be compatible with MCH; however, not all of them dissolved in oil (e.g. 1811, 1813).

Foam adjuvants were tested for compatibility with MCH at a ratio of MCH:excipient of 1:50. Isostearyl alcohol, Behenyl alcohol, Stearyl alcohol, Cetyl alcohol, Oleyl alcohol, Myristyl alcohol, Cetostearyl alcohol, Palmitic acid, Stearic acid and Oleic acid were found to be compatible with MCH. Isostearic acid was not compatible with MCH.

Additives were tested for compatibility with MCH at a ratio of MCH:excipient of 1:50. Aerosil and Menthol were found to be compatible with MCH. Titanium dioxide and Ethocel were not compatible with MCH.

Additives were tested for compatibility with MCH. Minimal quantities of water (100 μL) were added to MCH, suspended in excipients that had demonstrated compatibility to examine whether water can enhance oxidation/degradation in the absence or presence of antioxidant. In parallel, antioxidants were added to the MCH suspensions comprising water. Antioxidants were also added to excipients which were found to be non-compatible with MCH. Addition of water caused prompt degradation of MCH. Addition of the antioxidants alpha-tocopherol, BHA/BHT and propyl gallate did not prevent MCH degradation. Compatible excipients became incompatible in the presence of water. Addition of antioxidants did not alter this result.

Doxycycline

A similar compatibility study was conducted for Doxycycline Hyclate and Doxycycline Monohydrate.

The physicochemical properties of these two forms of Doxycycline are similar to those of other tetracycline antibiotics with the exception of differences resulting from the presence of an H₂O molecule in Doxycycline Monohydrate and an H₂O molecule and two HCl molecules for every water molecule in Doxycycline Hyclate.

General properties of Doxycycline Hyclate and Doxycycline Monohydrate:

Doxycycline Hyclate

1. Doxycycline Hyclate is a broad-spectrum antibiotic synthetically derived from oxytetracycline.

2. Doxycycline hyclate is a yellow crystalline powder soluble in water and in solutions of alkali hydroxides and carbonates.

3. Doxycycline hyclate has a high degree of lipid solubility and a low affinity for calcium binding.

Doxycycline Monohydrate

1. Doxycycline monohydrate is a broad-spectrum antibiotic synthetically derived from oxytetracycline.

2. The chemical designation of the light-yellow crystalline powder is alpha-6-deoxy-5-oxytetracycline.

The major degradative pathways for both types of Doxycycline are carbon-4 epimerization and oxidative processes.

Doxycycline is a member of the tetracycline antibiotics group and is commonly used to treat a variety of infections, particularly effective in treating acne condition.

Different compositions of hydrophilic and hydrophobic solvents containing Doxycycline Hyclate (Set I and Set II) and Doxycycline Monohydrate (Set III) were prepared by weighing the antibiotic in a glass vial and shaking overnight with each solvent investigated. Mixtures of Doxycycline salts 1.04% w/w with solid excipients were prepared in a similar way as for Minocycline HCl. The results are presented in Tables 22A-26.

TABLE 66
Doxycycline Hyclate Compatibility Test (Group I)
A

| | Mixtures of 1.04% w/w of Doxycycline Hyclate stored at 25° C., 40° C. and 50° C. for two weeks | | | | | | | | | |
|---|---|---|---|---|---|---|---|---|---|---|
| | Ingredients | | | | | | | | | |
| | | PPG-15 | | | | | | | | |
| | Cyclo- | stearyl | Octyl- | | Propylene | | | | | Diisopropyl |
| Group I | methicone | ether | dodecanol | Mineral oil | glycol | Glycerol | PEG 200 | PEG 400 | MCT oil | adipate |
| Visual | White | White | White | White | Light | Light | Light | Light | White | White |
| inspection | liquid and | liquid and | liquid and | liquid and | yellow | yellow | yellow | yellow | liquid and | liquid and |
| at T-0 | yellow | yellow | yellow | yellow | solution | solution | solution | solution | yellow | yellow |
| | powder | powder | powder | powder | | | | | powder | powder |
| | sedim. | sedim. | sedim. | sedim. | | | | | sedim. | sedim. |
| Visual | White | White | White | White | Light | Light | Light | Light | White | White |
| inspection | liquid and | liquid and | liquid and | liquid and | yellow | yellow | yellow | yellow | liquid and | liquid and |
| after the | yellow | yellow | yellow | yellow | solution | solution | solution | solution | yellow | yellow |
| storage at | powder | powder | powder | powder | | | | | powder | powder |
| 25° C. | sedim. | sedim. | sedim. | sedim. | | | | | sedim. | sedim. |
| Visual | White | White | Light | White | Yellow | brownish | Brown | Orange | White | White |
| inspection | liquid and | liquid and | orange | liquid and | solution | Yellow | solution | solution | liquid and | liquid and |
| after the | yellow | yellow | solution | yellow | | solution | | | yellow | yellow |
| storage at | powder | powder | | powder | | | | | powder | powder |
| 40° C. | sedim. | sedim. | | sedim. | | | | | sedim. | sedim. |
| Visual | White | White | Orange | White | Brownish | Light | Orange | Orange | White | White |
| inspection | liquid and | liquid and | solution | liquid and | orange | brown | solution | solution | liquid and | liquid and |
| after the | yellow | yellow | | yellow | solution | solution | | | yellow | yellow |
| storage at | powder | powder | | powder | | | | | powder | powder |
| 50° C. | sedim. | sedim. | | sedim. | | | | | sedim. | sedim. |
| Compat- | Compat. | Compat. | Non | Compat. | Non | Non | Non | Non | Compat. | Compat |
| ibility | no | no | compat. | no | compat. | compat. | compat. | compat. | no | no |
| Results | oxidation | oxidation | no | oxidation | oxidation | oxidation | oxidation | oxidation | oxidation | oxidation |
| after the | | | oxidation | | | | | | | |
| storage | | | | | | | | | | |

B

| | Mixtures of 1.04% w/w of Doxycycline Hyclate stored at 25° C., 40° C. and 50° C. for two weeks | | | | |
|---|---|---|---|---|---|
| | Ingredients | | | | |
| | Cetcaryl | | | Sorbitan | Dimethyl |
| Group I | octanoate | Hexylene glycol | Butylene glycol | Monolaurate | Isosorbide |
| Visual inspection | bright yellow | bright yellow | bright yellow | bright yellow | yellow solution |
| at T-0 | solution | solution | solution | mixture | |
| Visual inspection | bright yellow | bright yellow | bright yellow | Brown solution | Yellow solution |
| after the storage | solution | solution | solution | | |
| at 25° C. | | | | | |
| Visual inspection | bright yellow | light yellow | Light orange | Brown solution | Brownish orange |
| after the storage | solution | solution | solution | | |
| at 40° C. | | | | | |
| Visual inspection | White liquid and | Light yellow | Light orange | Black solution | Orange solution |
| after the storage | yellow powder | liquid and | solution | | |
| at 50° C. | sedim. | yellow powder | | | |
| | | sedim. | | | |
| Compatibility | Compat. no | Compat. no | Non compat. | Non compat. | Non compat. |
| Results | oxidation | oxidation | oxidation | oxidation | Oxidation |

Group II included Doxycycline Hyclate mixed with various vehicles with addition of antioxidants like alpha tocopherol, butylated hydroxytoluene (BHT), and ascorbic acid.

TABLE 23
Doxycycline Hyclate Compatibility Test (Group II)

| | Mixtures of 1.04% w/w of Doxycycline Hyclate stored at 25° C., 40° C. and 50° C. for two weeks | | | | |
|---|---|---|---|---|---|
| | Ingredients | | | | |
| | | | | Propylene glycol, alpha | PEG 200, alpha |
| | | | Ethanol 95%, BHT and | tocopherol and ascorbic | tocopherol and ascorbic |
| Group II | Ethanol 95% | Ethanol 95% and BHT | ascorbic acid | acid | acid |
| Visual inspection at | bright yellow | bright yellow | bright yellow | bright yellow | bright yellow |
| T-0 | solution | solution | solution | solution | solution |
| Visual inspection | bright yellow | bright yellow | Yellow solution | bright yellow | Yellow solution |
| after the storage at | solution | solution | | solution | |
| 25° C. | | | | | |
| Visual inspection | bright yellow | bright yellow | Yellow solution | Light orange solution | Orange solution |
| after the storage at | solution | solution | | | |
| 40° C. | | | | | |
| Visual inspection | bright yellow | bright yellow | Orange solution | Light organe solution | Brownish orange |
| after the storage at | solution | solution | | | solution |
| 50° C. | | | | | |
| Compatibility | compatible. | compatible | Non compatible. | Non compatible. | non compatible. |
| Results | no oxidation | no oxidation | Oxidation | oxidation | Oxidation |

TABLE 67
Doxycycline Hyclate Compatibility Test (Group III)

| | Mixtures of 1.04% w/w of Doxycycline Hyclate stored at 25° C., 40° C. and 50° C. for 3 days | | | | | | | | |
|---|---|---|---|---|---|---|---|---|---|
| | Ingredients | | | | | | | | |
| | | | | | Myristyl | | | | |
| | | | Steareth 20 | | alcohol and | | | | |
| | Isostearic | Oleyl | and | Hydrogenated | Stearyl | PEG 40 | PEG 100 | Sorbitan | |
| Group II | Acid | alchohol | Steareth 2 | Castor Oil | alcohol | Stearate | Stearate | Monostearate | Cocoglycerides |
| Visual | Yellow | Yellow | Yellow | Yellow | Yellow | Yellow | Yellow | Yellow | Yellow |
| inspection | suspen | suspen | suspen | suspen | suspen | suspen | suspen | suspen | suspen |
| at T-0 | | | | | | | | | |
| Visual | Yellow | Yellow | Yellow | Yellow | Yellow | Yellow | Yellow | Yellow | Yellow |
| inspection | suspen | suspen | suspen | suspen | suspen | suspen. | suspen | suspen | suspen |
| after the | | | | | | | | | |
| storage at | | | | | | | | | |
| 25° C. | | | | | | | | | |
| Visual | Yellow | Yellow | Brown | Yellow | Yellow | Brown | Yellow | Yellow | Yellow |
| inspection | suspen | suspen | suspen | suspen | suspen | suspen | suspen | suspen | suspen |
| after the | | | | | | | | | |
| storage at | | | | | | | | | |
| 40° C. | | | | | | | | | |
| Visual | Yellow | Yellow | Brown | Yellow | Yellow | Brown | Yellow | Yellow | Light |
| inspection | suspen | suspen | suspen | suspen | suspen | suspen | suspen | suspen | brown |
| after the | | | | | | | | | powder |
| storage at | | | | | | | | | |
| 50° C. | | | | | | | | | |
| Compat- | Compat. | Compat. | Non | Compat. | Compat. | Non | Compat.. | Compat. | Non |
| ibility | no | No | compat. | No | No | Compat. | No | No | Compat. |
| Results | oxidation | oxidation | Oxidation | oxidation | oxidation | oxidation | oxidation | oxidation | oxidation |

Suspen.—suspension;
compat.—compatible

A similar compatibility test was performed on another form of Doxycycline-Doxycycline Monohydrate. The results are presented in Tables 25A, 25B, and 26.

TABLE 68
Doxycycline Monohydrate Compatibility Test (Group I)
A

| | Mixtures of 1.04% w/w of Doxycycline Monohydrate stored at 25° C., 40° C., and 50° C. for two weeks | | | | | | | | | |
|---|---|---|---|---|---|---|---|---|---|---|
| | Ingredients | | | | | | | | | |
| | | PPG-15 | | | | | | | | |
| | Cyclo- | stearyl | Octyl- | | Propylene | | | | | Diisopropyl |
| Group I | methicone | ether | dodecanol | Mineral oil | glycol | Glycerol | PEG 200 | PEG 400 | MCT oil | adipate |
| Visual | White | White | White | White | Light | yellow | yellow | Dark | White | White |
| inspection | liquid and | liquid and | liquid and | liquid and | yellow | solution | solution | yellow | liquid and | liquid and |
| at T-0 | yellow | yellow | yellow | yellow | solution | | | solution | yellow | yellow |
| | powder | powder | powder | powder | | | | | powder | powder |
| | sedim. | sedim. | sedim. | sedim. | | | | | sedim. | sedim. |
| Visual | White | White | White | White | Orange | yellow | Yellowish | Yellowish | White | White |
| inspection | liquid and | liquid and | liquid and | liquid and | solution | solution | black | brown | liquid and | liquid and |
| after the | yellow | yellow | yellow | yellow | | | solution | solution | yellow | yellow |
| storage at | powder | powder | powder | powder | | | | | powder | powder |
| 25° C. | sedim. | sedim. | sedim. | sedim. | | | | | sedim. | sedim. |
| Visual | White | Yellowish | orange | White | Black | black | Black | Brown | White | White |
| inspection | liquid and | orange | solution | liquid and | solution | solution | solution | solution | liquid and | liquid and |
| after the | yellow | mixture | | yellow | | | | | yellow | yellow |
| storage at | powder | | | powder | | | | | powder | powder |
| 40° C. | sedim. | | | sedim. | | | | | sedim. | sedim. |
| Visual | White | Yellowish | Orange | White | black | black | Black | Black | Dirty | Brown |
| inspection | liquid and | orange | solution | liquid and | solution | solution | solution | solution | yellow | mixture |
| after the | yellow | mixture | | yellow | | | | | | |
| storage at | powder | | | powder | | | | | | |
| 50° C. | sedim. | | | sedim. | | | | | | |
| Compatibility | Compat. | Non | Non | Compat. | Non | Non | Non | Non | Non | Non |
| Results | no | compat. | compat. | no | compat. | compat. | compat. | compat. | compat. | compat. |
| after the | oxidation | oxidation | oxidation | oxidation | oxidation | oxidation | oxidation | oxidation | oxidation | oxidation |
| storage | | | | | | | | | | |

B

| | Mixtures of 1.04% w/w of Doxycycline Monohydrate stored at 25° C., 40° C., and 50° C. for two weeks | | | | |
|---|---|---|---|---|---|
| | Ingredients | | | | |
| | Cetearyl | | | Sorbitan | Dimethyl |
| Group I | octanoate | Hexylene glycol | Butylene glycol | Monolaurate | Isosorbide |
| Visual inspection | White liquid and | White liquid and | White liquid and | Brown mixture | yellow solution |
| at T-0 | yellow powder | yellow powder | yellow powder | | |
| | sedim. | sedim. | sedim. | | |
| Visual inspection | White liquid and | bright yellow | Orange solution | orange solution | orange solution |
| after the storage | yellow powder | solution | | | |
| at 25° C | sedim. | | | | |
| Visual inspection | White liquid and | Brownish black | Brownish black | Brown solution | orange solution |
| after the storage | yellow powder | solution | solution | | |
| at 40° C. | sedim. | | | | |
| Visual inspection | White liquid and | Black solution. | black solution | brown solution | Orange solution |
| after the storage | yellow powder | | | | |
| at 50° C. | sedim. | | | | |
| Compatibility | compat. no | Non compat. | Non compat. | Non compat. | non compat. |
| Results | oxidation | oxidation | Oxidation | oxidation | Oxidation |

TABLE 69
Doxycycline Monohydrate Compatibility Test (Group II)

| | Mixtures of 1.04% w/w of Doxycycline Monohydrate stored at 25° C., 40° C. and 50° C. for two weeks | | | | |
|---|---|---|---|---|---|
| | Ingredients | | | | |
| | | | | Propylene glycol, alpha | PEG 200, alpha |
| | | Ethanol | Ethanol 95%, BHT and | tocopherol and ascorbic | tocopherol and ascorbic |
| Group II | 95% | 95% and BHT | ascorbic acid | acid | acid |
| Visual inspection at | bright yellow | bright yellow | bright yellow | bright yellow | bright yellow |
| T-0 | solution | solution | solution | solution | solution |
| Visual inspection | Brown solution | Brown solution | orange solution | Yellowish orange | Yellow solution |
| after the storage at | | | | solution | |
| 25° C. | | | | | |
| Visual inspection | Brown solution | Brown solution | Orange solution | Orange solution | Dark yellow solution |
| after the storage at | | | | | |
| 40° C. | | | | | |
| Visual inspection | Black solution | Black solution | Black solution | Brownish orange | Brown orange solution |
| after the storage at | | | | solution | |
| 50° C. | | | | | |
| Compatibility | Non compatible. | Non compatible | Non compatible. | Non compatible. | non compatible. |
| Results | oxidation | oxidation | Oxidation | oxidation | Oxidation |

Interesting and unexpected phenomena were found during the compatibility studies of Minocycline HCl, Doxycycline Hyclate and Doxycycline Monohydrate:

1. Whilst Minocycline displayed intensive oxidation on dissolution in glycerol, the antibiotic surprisingly revealed full compatibility with octyldodecanol, a branched chain fatty alcohol. Both molecules have similar hydroxyl units in their structures.

2. Doxycycline Hyclate and Monohydrate unexpectedly revealed different compatibility with excipients. For example, Doxycycline Hyclate was stable in a mixture with PPG-15 Stearyl Ether. Surprisingly, the Doxycycline Monohydrate was found to be non-compatible with PPG-15 Stearyl Ether during the storage at 40° C. and 50° C. for two weeks.

3. Doxycycline Hyclate was stable in a mixture with ethanol 95% and hexylene glycol. Doxycycline Monohydrate oxidized in similar mixtures.

4. Unexpectedly, addition of strong anti-oxidants like alpha-tocopherol and ascorbic acid did not prevent the oxidation of any of Minocycline HCl, Doxycycline Hyclate and Monohydrate in a waterless medium of propylene glycol and PEG 200.

5. Surprisingly, Doxycycline Hyclate revealed stability in Ethanol 95% following the storage at 40° C. and 50° C. for two weeks although both Minocycline HCl and Doxycycline Monohydrate changed their colour from yellow to orange upon dissolution in Ethanol 95%.

6. In conclusion, the following non predictable substances were found to be compatible with Minocycline and Doxycycline:

TABLE 70
Summary of MCH and DOX compatibility studies

| | Compatibility tested after the storage for up to 3 weeks | | | |
|---|---|---|---|---|
| | Minocycline | Doxycycline | Doxycycline | |
| Ingredient | HCl | Hyclate | Monohydrate | Comments |
| Cyclomethicone 5 NF | Yes | Yes | Yes | All compatible |
| PPG-15 Stearyl Ether | Yes | Yes | No | |
| Octyldodecanol | Yes | No | No | |
| Mineral Oil | Yes | Yes | Yes | All compatible |
| Propylene Glycol | No | No | No | |
| Glycerol | No | No | No | |
| PEG 200 | No | No | No | |
| PEG 400 | No | No | No | |
| MCT Oil | Yes | Yes | No | |
| Diisopropyl adipate | Yes | Yes | No | |
| Ethanol 95% | No | Yes | No | |
| Isostearic acid | No | Yes | Not tested | |
| Oleyl alcohol | Yes | Yes | Not tested | |
| Steareth 20 (Polyoxyl 20 | No | No | Not tested | |
| Stearyl Ether) | | | | |
| Steareth 2 (Polyoxyl 2 | No | No | Not tested | |
| Stearyl Ether) | | | | |
| Methyl glycose | No | Not tested | Not tested | |
| sesquistearate (MGSS) | | | | |
| Aluminum Starch | Yes | Not tested | Not tested | |
| Octenylsuccinate(ASOS) | | | | |
| Cetearyl octanoate | Yes | Yes | Yes | All compatible |
| Hydrogenated Castor Oil | Yes | Yes | Not tested | |
| Stearyl alcohol | Yes | Yes | Not tested | |
| Myristyl alcohol | Yes | Yes | Not tested | |
| Titanium Dioxide | Yes | Not tested | Not tested | |
| PEG 40 stearate | Yes | No | Not tested | |
| PEG 100 Stearate | Yes | Yes | Not tested | |
| Sorbitan Monostearate | Yes | Yes | Not tested | |
| Cocoglycerides | Yes | No | Not tested | |
| Coconut Alcohcol | Yes | Not tested | Not tested | |
| Hexylene glycol | No | Yes | No | |
| Butylene glycol | No | No | No | |
| Sorbitan Monolaurate | No | No | No | |
| Dimethyl Isosorbide | No | No | No | |
| Titanium dioxide | Yes | Not tested | Not tested | |
| Methyl glycose | No | Not tested | Not tested | |
| sesquistearate (MGSS) | | | | |
| Aluminum Starch | Yes | Not tested | Not tested | |
| Octenylsuccinate (ASOS) | | | | |
| Coconut alcohol | Yes | Not tested | Not tested | |

7. As could be seen from Table 70, not all of the ingredients compatible with MCH are compatible with Doxycycline Hyclate or Monohydrate. For example, octyldodecanol is compatible with Minocycline HCl but revealed incompatibility with Doxycycline Hyclate and Monohydrate. Surprisingly, there are discrepancies in the list of ingredients compatible with Doxycycline Hyclate and Doxycycline Monohydrate: for example, PPG-15 Stearyl Ether is compatible with Doxycycline Hyclate and non-compatible with Doxycycline Monohydrate.

8. The data presented herein could be used for selection of active materials from tetracycline family for topical formulations. A list of ingredients that were found to be compatible with MCH and DOX could be applied to other antibiotics from the tetracycline family. The following ingredients are suitable for topical formulations: mineral oil, cyclomethicone, cetearyl octanoate. Few ingredients are compatible with both forms of doxycycline and are also compatible with minocycline.

### Example 6—Phase II Study for FDX104 (Doxycycline Foam) in EGFRI Induced or Associated Rash

Twenty-four subjects receiving targeted monoclonal antibody treatment for colon and head and neck cancers were randomized and received at least one dose of FDX104 (4% doxycycline; Example 2, Table 2D) in a multicenter, randomized, double-blind, placebo-controlled, Phase 2 clinical study to evaluate the safety, tolerability, and efficacy of FDX104 for the prophylactic treatment of EGFRI-induced skin toxicity. Specifically, the subjects in the study were receiving epidermal growth factor receptor antibody inhibitors (EGFRI) including cetuximab (Erbitux®, Eli Lilly) and panitumumab (Vectibix®, Amgen). EGFRIs are known to induce moderate-to-severe skin rash in patients. The rash, also referred to as acneiform (acne-like) rashes, are the most common side effect of EGFRI drugs, and they can severely impact a patient's physical, psychological, and social well-being and can lead to treatment discontinuation and dose reduction. According to the prescription information of cetuximab and panitumumab, upon the occurrence of severe rash, the dosing of the EGFRI drug should be withheld, reduced or discontinued.

Twenty-four subjects applied FDX104 and vehicle twice daily, for 5 weeks. To optimize the power of the study, each subject acted as their own control by treating one side of the face with FDX104 and the other side with the matching foam vehicle (Placebo), (Example 2, Table 2D) in a blinded and randomized manner. The FDX104 treatment was started 7 (±3) days prior to EGFRI therapy. Subjects returned for evaluation at 2 weeks and 4 weeks following initiation of EGFRI therapy, i.e., after 3 weeks and 5 weeks of FDX104 treatment (end-of-treatment). Final follow-up was performed at 8 weeks following initiation of EGFRI treatment (i.e., 9 weeks after starting FDX104 treatment). High-definition photographs of the front and each side of the face were taken at each study visit (i.e., baseline, Week 2 and Week 4 of EGFRI treatment), and were used for rash severity grading, which was assessed blindly by an independent dermatologist at the end of the study, using established grading scales for evaluating the rash associated with the EGFRI treatment. Rash severity was assessed using the Global Severity Scale (GSS), which has four severity grades: 0=None; 1=Mild; 2=Moderate; and 3=Severe (also known as the Scope scale; Scope A. et al. “A prospective randomized trial of topical pimecrolimus for cetuximab-associated acnelike eruption.”). Severity was also assessed by the study investigators at each study visit using the modified MASCC EGFR Inhibitor Papulopustular Eruption Grading Scale (MESTT) (see Lacouture M A et al. (2011) “Clinical practice guidelines for the prevention and treatment of EGFR inhibitor-associated dermatologic toxicities,” Support Care Cancer 19:1079-1095). The maximal score for each subject was used for the analyses, unless otherwise stated.

Twenty of the twenty-four patients in the randomized intent-to-treat (ITT) population completed the study. Adherence with treatment was high, with a mean of 97.5% of subjects consistently using treatment as directed. Baseline characteristics of the enrolled subjects are shown in Table 71 below.

TABLE 71
Baseline characteristics of enrolled subjects

| Characteristic | |
|---|---|
| Mean age, years (range) | 55.2 (24.9-78.0) |
| Gender, n (%) | |
| Male/Female | 15 (62.5)/9 (37.5) |
| Race, n (%) | |
| Caucasian | 24 (100) |
| Mean MCRC duration, months (SD) | 17.9 (23.1) |
| EGFRI treatment, n | |
| Cetuximab | 12 |
| Panitumumab | 11* |

*One subject included in the ITT population, who was randomized to FDX104, withdrew from the study before receiving the EGFR1 treatment due to use of a prohibited medication.

Overall, 79% ( 19/24) of the subjects developed rash and 58% ( 14/24) developed moderate/severe rash (GSS) on at least one side of the face within 4 weeks of starting EGFRI treatment. Severe rash (grade 3) developed in 9/24 subjects (37.5%) on the vehicle (placebo) side compared with only 4/24 subjects (16.7%) on the FDX104 side. Five of twenty-four subjects did not develop a severe rash with FDX104. The GSS maximum scores for individual subjects are indicated in Table 72, below.

TABLE 72
GSS maximum scores for individual subjects

| | Score (GSS) | | | | | | | | | | | | | | | | | | | | | | | |
|---|---|---|---|---|---|---|---|---|---|---|---|---|---|---|---|---|---|---|---|---|---|---|---|---|
| | No rash (n = 5) | | | | | Mild rash (n = 5) | | | | | Moderate or severe rash (n = 14) | | | | | | | | | | | | | |
| FDX104 | 0 | 0 | 0 | 0 | 0 | 1 | 1 | 1 | 1 | 1 | 2 | 1 | 1 | 3 | 3 | 2 | 1 | 1 | 2 | 1 | 2 | 2 | 3 | 3 |
| Vehicle | 0 | 0 | 0 | 0 | 0 | 1 | 1 | 1 | 1 | 1 | 2 | 3 | 3 | 3 | 3 | 2 | 2 | 3 | 1 | 3 | 3 | 2 | 3 | 3 |
| Score | 0 | 0 | 0 | 0 | 0 | 0 | 0 | 0 | 0 | 0 | 0 | 2 | 2 | 0 | 0 | 0 | 1 | 2 | −1 | 2 | 1 | 0 | 0 | 0 |
| difference | | | | | | | | | | | | | | | | | | | | | | | | |

The results of this study indicate that FDX104 can prevent the development of or treat moderate-to-severe skin rashes in patients treated with EGFRIs.

When evaluating the tested patient population (those who developed and those who did not develop a rash), a clear treatment benefit was observed. Specifically, the mean and median severity scores following FDX104 treatment were lower compared with the placebo (median grade 1 vs 2, respectively). Using the GSS scale, the severity of the rash on the FDX104 side vs. the vehicle side was 1.3 vs. 1.7, and using the MESTT scale was 1.6 vs. 1.9. In addition, the FDX104 and placebo groups showed a statistically significant difference in the severity of the antibody induced rash, (T-test two sided p=0.036) (See Table 73, below).

TABLE 73
Mean maximal rash grade (ITT analysis)

| | Maximal GSS | | Maximal MESTT | |
|---|---|---|---|---|
| | grade (n = 24) | | grade (n = 24) | |
| | FDX104 | Vehicle | FDX104 | Vehicle |
| Mean severity (SD) | 1.3 (1.01) | 1.7 (1.20) | 1.6 (1.13) | 1.9 (1.19) |
| Median severity | 1.0 | 2.0 | 2.0 | 2.0 |
| Wilcoxon signed-rank | 0.63 | — | 0.344 | — |
| P-value | | | | |
| P Value (ITT, t-test, | 0.0359 | | | |
| paired): | | | | |

The paired differences between treatment groups (ITT) were transformed to a ranked difference based on clinical importance, as shown in Table 74. Analysis of the GSS scale results showed a significant difference in favor of FDX104 over vehicle (P=0.047; Wilcoxon signed-rank test). A similar trend was observed for the MESTT scale (data not shown).

TABLE 74
Paired ranked differences between treatment groups (ITT analysis)

| | Overall | |
|---|---|---|
| | (n = 24) | Ranked |
| Superior treatment difference in grade (GSS) | n (%) | difference |
| FDX104 Superior | | |
| +3: Vehicle = 3, FDX104 = 0 | 0 | +4 |
| +2: Vehicle = 3, FDX104 = 1; Vehicle = 2; | 4 (16.7) | +3 |
| FDX104 = 0 | | |
| +1: Vehicle = 3, FDX104 = 2 | 1 (4.2) | +2 |
| +1: Vehicle = 2, FDX104 = 1; Vehicle = 1, | 1 (4.2) | +1 |
| FDX104-0 | | |
| None Superior | | |
| 0 | 17 (70.8) | 0 |
| Vehicle Superior | | |
| −1: Vehicle = 1, FDX104 = 2; Vehicle = 0, | 1 (4.2) | −1 |
| FDX104 = 1 | | |
| −1: Vehicle = 2, FDX104 = 3 | 0 | −2 |
| −2: Vehicle = 1, FDX104 = 3; Vehicle = 0, | 0 | −3 |
| FDX104 = 2 | | |
| −3: Vehicie = 0, FDX104 = 3 | 0 | −4 |
| P-value, Wilcoxon signed-rank | 0.047 | |

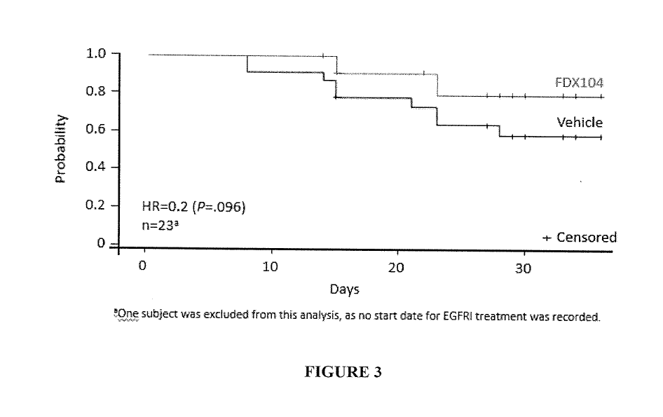

FDX104 treatment also showed a trend for a higher probability of a subject remaining free of severe rash (<3, GSS) after the start of EGFRI treatment (See FIG. 3). The probability of remaining free of severe rash from the start of EGFRI treatment to the time point of developing a grade 3 rash (GSS), i.e., the hazard ratio for FDX104/vehicle, was 0.2 (P=0.096): This suggested a higher possibility of developing a grade 3 rash with the vehicle.

In the Response Population (i.e., those subjects with grade 2 or grade 3 rash on at least one side of the face), the mean maximal rash grade on the FDX104 side was lower (i.e., better) than on the vehicle side, for both GSS and MESTT scales, as shown in Table 75, below.

TABLE 75
Mean maximal rash grade (Response Population)

| | Maximal GSS grade | | Maximal MESTT grade | |
|---|---|---|---|---|
| | (n = 14) | | (n = 17) | |
| | FDX104 | Vehicle | FDX104 | Vehicle |
| Mean (SD) | 1.9 (0.83) | 2.6 (0.65) | 2.1 (0.93) | 2.5 (0.62) |
| Median | 2.0 | 3.0 | 2.0 | 3.0 |
| Wilcoxon signed- | 0.063 | — | 0.250 | — |
| rank P-value | | | | |

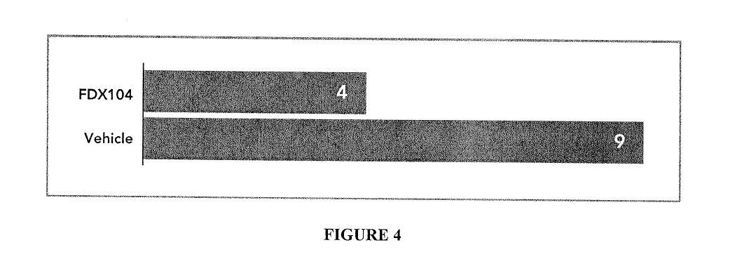

When analyzing a sub-group of the more severe patient population (n=14 patients), i.e., those subjects who developed a clinically significant rash (i.e., ≥2 Scope scale), additional findings of the study indicated that FDX104 is useful in preventing severe acneiform rash (see FIG. 4). Specifically, FDX104 prevented the development of or reduced the severity of such a rash in more than forty percent of the cases (6 of 14 subjects, 43%). Moreover, in 7 of the 14 patients, there was no worsening (change in severity) observed as compared to control. On the contrary, only one patient (7%) showed a moderately improved condition on the placebo treated side of the face compared to the drug treated side of the face. The mean and median severity scores following FDX104 treatment were lower compared with the placebo (median grade 2 vs 3, respectively). The FDX104 and placebo group-treated areas showed a statistically significant difference in the severity of the antibody induced rash (T-test two sided, p=0.035). (See Table 76, below). Whereas 9 patients developed the more severe rash (grade 3) on the placebo treated side of the face (64%), in 5 of said 9 patients only a grade 1 (mild) rash was observed in the FDX104 treated sides, i.e., FDX104 prevented the development of the severe rash in 55% of the patients. See Table 75, above.

TABLE 76
Efficacy results of patients with moderate-severe rash (N = 14) based on
photograph evaluation by an independent dermatologist (Scope A Scale)

| Patient Number | FDX104 treated side | Placebo treated side | Difference |
|---|---|---|---|
| 1 | 2 | 2 | 0 |
| 11 | 1 | 3 | 2 |
| 16 | 1 | 3 | 2 |
| 17 | 3 | 3 | 0 |
| 18 | 3 | 3 | 0 |
| 20 | 2 | 2 | 0 |
| 21 | 1 | 2 | 1 |
| 25 | 1 | 3 | 2 |
| 26 | 2 | 1 | −1 |
| 7 | 1 | 3 | 2 |
| 10 | 2 | 3 | 1 |
| 12 | 2 | 2 | 0 |
| 22 | 3 | 3 | 0 |
| 24 | 3 | 3 | 0 |

For those subjects who developed a clinically significant rash, FDX104 prevented the development or severity of such a rash in more than 40% of the cases (see FIG. 4). The population was representative (i.e., the percentage developing a clinically significant rash was similar to the published data; see, e.g., Lacouture et al., Supportive Care Cancer (2011) 19(8): 1079-1095). For those patients who developed a clinically significant rash (grade 2 or higher) in this study, the odds of developing a more severe rash when on the placebo were 7 times higher than developing such a rash when on FDX104. For those patients who developed a clinically significant rash (grade 2 or higher) in this study, the odds of developing a more severe rash when on the FDX104 were about 7 times lower than developing such a rash when on placebo. Circles indicate a successful response to FDX104.

Throughout the study, adverse events (including systemic and dermal) were assessed using CTC-AE v4.0. FDX104 was found to be safe and well-tolerated in the subjects receiving EGFRIs. No systemic drug-related adverse events were recorded. Table 77, below, summarizes the FDX104 safety profile. Overall, 20 subjects experienced a total of 68 adverse events (AEs). The most common AEs were oral mucositis (29.2%), nausea and vomiting (20.8% each), and pruritus (16.7%), which were well-known to be related to EGFRI treatment. Six treatment-related AEs developed in 5 subjects; all were mild, local dermal skin reactions, and most ( 4/6) had resolved by study completion. One serious AE, febrile neutropenia, developed in 1 subject, and was considered unrelated to the study drug.

TABLE 77
FDX104 safety profile

| | N = 24 |
|---|---|
| Subjects with serious AEs, n (%) | 1* (4.2) |
| Subjects with any drug-related AE, n (%) | 5 (20.8) |
| Pruritus | 2 (8.3) |
| Skin hypopigmentation | 1 (4.2) |
| Dryness around the mouth and nose | 1 (4.2) |
| Erythema of face after application of foam | 1 (4.2) |
| Facial pain | 1 (4.2) |
| Discontinuation due to drug-related toxicity, n (%) | 0 |

*One case of febrile neutropenia, which was considered unrelated to treatment

The results indicate that FDX104 can reduce the incidence and severity of rash. Current treatments include prophylactic oral medication that potentially entails systemic side effects; thus, doctors do not have an effective treatment for cancer therapy side effects, which is especially disturbing and disruptive to the cancer patient population. There is a major unmet need for a safe and effective treatment for anti-cancer treatment induced rash. FDX104 has the potential to improve patients' quality of life and help maintain patients on their optimum anti-cancer treatment.

Brief highlights include, e.g., topical tetracycline antibiotic can help patients receiving EGFRI treatment. 4% doxycycline foam appeared to be safe and well tolerated in subjects who received EGFRI treatment with cetuximabor panitumumab. No drug-related systemic side effects were reported. 4% doxycycline foam appeared to be superior to vehicle in preventing EGFRI induced skin toxicity. More subjects had a lower grade of rash on the side of the face treated with 4% doxycycline foam than on the vehicle-treated side. The most prominent effect appeared to occur in the proportion of subjects who developed a severe grade rash on the vehicle treated side. Compliance with FDX104 was high, at 97.5%. Topical FDX104 has the potential to improve patients' quality of life, their adherence to EGFRI treatment, and cancer outcomes.

In some embodiments a similar Phase II clinical studies for additional tetracycline antibiotic formulations such as DOX331, DOX332, DOD-003, MCD-037, MCD-045, MCD-052 MCD-053 and MCD-058 is undertaken.

In some embodiments, a Phase II study for other tetracycline antibiotic formulations (such as DOX331, DOX332, DOD-003, MCD-037, MCD-045, MCD-052 MCD-053 and MCD-058) in EGFRI induced or associated rash are not dissimilar to that for the FDX104 formulation.

In some embodiments, a Phase II clinical study indicates that other tetracycline antibiotic formulations (such as DOX331, DOX332, DOD-003, MCD-037, MCD-045, MCD-052 MCD-053 and MCD-058) can prevent the development of or treat moderate-to-severe skin rashes in patients treated with EGFRIs.

In some embodiments, the mean and median severity scores in a Phase II clinical study for other tetracycline antibiotic formulations (such as DOX331, DOX332, DOD-003, MCD-037, MCD-045, MCD-052 MCD-053 and MCD-058) are following treatment lower compared with those with a placebo.

In some embodiments, there is a trend for a higher probability of a subject remaining free of severe rash (<3, GSS) after the start of EGFRI treatment with treatment by other tetracycline antibiotic formulations (such as DOX331, DOX332, DOD-003, MCD-037, MCD-045, MCD-052 MCD-053 and MCD-058). In an embodiment there is a higher possibility of developing a grade 3 rash with the vehicle.

In some embodiments, in a Response Population (i.e., those subjects with grade 2 or grade 3 rash on at least one side of the face), the mean maximal rash grade on the treated side (treated by one of the other tetracycline antibiotic formulations (such as DOX331, DOX332, DOD-003, MCD-037, MCD-045, MCD-052 MCD-053 and MCD-058)) is lower (i.e., better) than on the vehicle side, for both GSS and MESTT scales.

In some embodiments, when analyzing a sub-group of a more severe patient population i.e., those subjects who develop a clinically significant rash (i.e., ≥2 Scope scale), other tetracycline antibiotic formulations (such as DOX331, DOX332, DOD-003, MCD-037, MCD-045, MCD-052 MCD-053 and MCD-058) are useful in preventing severe acneiform rash. In an embodiment treatment can prevent the development of or reduces the severity of such a rash in more than about 40% of the cases. For example, prevention is in more than about 45% of the cases, or more than about 50% of the cases, or more than about 55% of the cases, or more than about 60% of the cases, or more than about 65% of the cases, or more than about 70% of the cases, or more than about 75% of the cases, or more than about 80% of the cases, or more than about 85% of the cases, or more than about 90% of the cases, or more than about 95% of the cases. In some embodiments mean and median severity scores following treatment with other tetracycline antibiotic formulations (such as DOX331, DOX332, DOD-003, MCD-037, MCD-045, MCD-052 MCD-053 and MCD-058) are lower compared with a placebo. In some embodiments a tetracycline antibiotic and a placebo group-treated areas can show a statistically significant difference in the severity of the antibody induced rash. In some embodiments other tetracycline antibiotic formulations (such as DOX331, DOX332, DOD-003, MCD-037, MCD-045, MCD-052 MCD-053 and MCD-058) can also prevent the development of the severe rash in about 55% of the patients. In some embodiments the development of the severe rash can be prevented in about 30% of the patients, or in about 35% of the patients, or in about 40% of the patients, or in about 45% of the patients, or in about 50% of the patients, or in about 55% of the patients, or in about 60% of the patients, or in about 65% of the patients, or in about 70% of the patients, or in about 75% of the patients, or in about 80% of the patients, or in about 85% of the patients, or in about 90% of the patients, or in about 95% of the patients.

In some embodiments, for those patients who develop a clinically significant rash (grade 2 or higher), the odds of developing a more severe rash when on the placebo are 7 times higher than developing such a rash when on other tetracycline antibiotic formulations (such as DOX331, DOX332, DOD-003, MCD-037, MCD-045, MCD-052 MCD-053 and MCD-058). In other embodiments, the odds are about 20 times higher, or about 18 times higher, or about 18 times higher, or about 16 times higher, or about 14 times higher, or about 12 times higher, or about 10 times higher, or about 8 times higher, or about 6 times higher, or about 4 times higher, or about 2 times higher.

In some embodiments, for those patients who develop a clinically significant rash (grade 2 or higher), the odds of developing a more severe rash when on other tetracycline antibiotic formulations (such as DOX331, DOX332, DOD-003, MCD-037, MCD-045, MCD-052 MCD-053 and MCD-058) are about 7 times lower than developing such a rash when on placebo. In other embodiments, the odds are about 20 times lower, or about 18 times lower, or about 18 times lower, or about 16 times lower, or about 14 times lower, or about 12 times lower, or about 10 times lower, or about 8 times lower, or about 6 times lower, or about 4 times lower, or about 2 times lower.

In some embodiments, other tetracycline antibiotic formulations (such as DOX331, DOX332, DOD-003, MCD-037, MCD-045, MCD-052 MCD-053 and MCD-058) are safe and well-tolerated in the subjects receiving EGFRIs. In some embodiments no systemic drug-related adverse events are recorded. In some embodiments The oral mucositis, nausea and vomiting, and pruritus, which are well-known to be related to EGFRI treatment are common adverse events.

In some embodiments, a Phase II clinical study for other tetracycline antibiotic formulations such as DOX331, DOX332, DOD-003, MCD-037, MCD-045, MCD-052 MCD-053 and MCD-058 indicate that such formulations can reduce the incidence and severity of rash. DOX331, DOX332, DOD-003, MCD-037, MCD-045, MCD-052 MCD-053 and MCD-058 have the potential to improve patients' quality of life and help maintain patients on their optimum anti-cancer treatment.

In some embodiments, DOX331, DOX332, DOD-003, MCD-037, MCD-045, MCD-052 MCD-053 and MCD-058 may help patients receiving EGFRI treatment. In some embodiments, DOX331, DOX332, DOD-003, MCD-037, MCD-045, MCD-052 MCD-053 and MCD-058 are safe and well tolerated in subjects who receive EGFRI treatment with cetuximabor panitumumab. In some embodiments there are no drug-related systemic side effects. DOX331, DOX332, DOD-003, MCD-037, MCD-045, MCD-052 MCD-053 and MCD-058 are in some embodiments superior to a vehicle in preventing EGFRI induced skin toxicity. In some embodiments more subjects can have a lower grade of rash on the side of the face treated with DOX331, DOX332, DOD-003, MCD-037, MCD-045, MCD-052 MCD-053 and MCD-058 than on the vehicle-treated side. In some embodiments subjects who develop a severe grade rash on the vehicle treated side are able to respond better to treatment and a prominent effect can occur on with treatment. In some embodiments compliance with DOX331, DOX332, DOD-003, MCD-037, MCD-045, MCD-052 MCD-053 and MCD-058 is high. In some embodiments application with one or more of topical DOX331, DOX332, DOD-003, MCD-037, MCD-045, MCD-052 MCD-053 and MCD-058 can improve patients' quality of life, their adherence to EGFRI treatment, and cancer outcomes.

1-79. (canceled)

80. A method for treating acne vulgaris in a subject in need thereof, comprising administering to the subject a topical composition comprising an effective amount of a minocycline antibiotic in a carrier according to a dosing schedule such that
(a) the mean maximum plasma concentration of the tetracycline antibiotic is about 0.2 ng/mL to about 5 ng/mL after at least one day of use, and/or
(b) the mean maximum plasma concentration of the tetracycline antibiotic is about 0.2 ng/mL to about 12 ng/mL after at least 16 days of use, and/or
(c) the area under the plasma concentration versus time curve at the last time point with a detectable drug concentration equal to or greater than the limit of quantification (AUCT) is about 36 ng*h/mL to 132 ng*h/mL,
wherein the carrier is essentially waterless and comprises at least one hydrophobic solvent, at least one wax, at least one fatty alcohol, and at least one fatty acid, and wherein the carrier is combined with propellant to produce a foam.

81. The method according to claim 80, wherein the at least one hydrophobic solvent is about 60% to about 95% by weight of the carrier, wherein the at least one wax is about 0.6% to about 5% by weight of the carrier, wherein the at least one fatty alcohol is about 3% to about 10% by weight of the carrier, and wherein the at least one fatty acid is about 0.7% to about 4% by weight of the carrier.

82. The method according to claim 80, wherein the hydrophobic solvent comprises a mineral oil, a silicone oil, a coconut oil and a soybean oil, wherein the wax comprises hydrogenated castor oil and a beeswax, wherein the fatty alcohol comprises a stearyl alcohol, a behenyl alcohol, a cetostearyl alcohol, and a myristyl alcohol, and wherein the fatty acid comprises a stearic acid.

83. The method according to claim 80, wherein the carrier comprises:
about 48% to about 51% soybean oil by weight of the topical composition,
about 23% to about 25% coconut oil by weight of the topical composition,
about 4% to about 6% cyclomethicone by weight of the topical composition,
about 0.7% to about 2% light mineral oil by weight of the topical composition,
about 3% to about 4% cetostearyl alcohol by weight of the topical composition,
about 2% to about 4% stearic acid by weight of the topical composition,
about 2% to about 3% myristyl alcohol by weight of the topical composition,
about 1% to about 3% hydrogenated castor oil by weight of the topical composition,
about 1% to about 3% beeswax by weight of the topical composition,
about 1% to about 2% stearyl alcohol by weight of the topical composition,
about 0.5% to about 1.5% behenyl alcohol by weight of the topical composition.

84. The method according to claim 80, wherein the minocycline antibiotic is minocycline or salt, hydrate, crystal, polymorph, enantiomer, isomer, ion, or complex thereof.

85. The method according to claim 80, wherein the minocycline antibiotic is minocycline or a salt thereof.

86. The method according to claim 85, wherein the minocycline or salt thereof is minocycline hydrochloride.

87. The method according to claim 86, wherein the effective amount of minocycline hydrochloride is about 0.1% w/w to about 10% w/w of the topical composition.

88. The method according to claim 86, wherein the effective amount of minocycline hydrochloride is about 1% w/w to about 6% w/w of the topical composition.

89. The method according to claim 86, wherein the effective amount of minocycline hydrochloride is about 4% w/w of the topical composition.

90. The method according to claim 85, wherein the mean maximum plasma concentration of the minocycline or salt thereof after administering the topical composition is at least 50 times less than the mean maximum plasma concentration after a comparable administration of an oral minocycline.

91. The method according to claim 85, wherein the mean maximum plasma concentration of the minocycline or salt thereof after administering the topical composition is at least 500 times less than the mean maximum plasma concentration after a comparable administration of an oral minocycline.

92. The method according to claim 85, wherein the mean maximum concentration of the minocycline or salt thereof in the plasma is reached during the 24 hour period following administration.

93. The method according to claim 80, wherein the topical composition is administered for at least 16 days.

94. The method according to claim 85, wherein the mean maximum concentration of the minocycline or salt thereof in the plasma is between 0.2 ng/mL and 3.0 ng/mL after one day of use.

95. The method according to claim 85, wherein the mean maximum concentration of the minocycline or salt thereof in the plasma is between 0.2 ng/mL and 7.0 ng/mL after sixteen days of use.

96. The method of claim 90, wherein the comparable oral minocycline is at a dose of about 100 mg to 135 mg.

97. The method according to claim 80, wherein the dosing schedule is topical application at least once daily for at least 16 days at an effective amount of minocycline hydrochloride.

98. The method according to claim 80, wherein the propellant is a pressurized gas mixture.

99. The method according to claim 80, wherein the gas mixture is a hydrocarbon gas.

100. The method according to claim 80, further comprising a retinoid.

101. The method according to claim 80, wherein the hydrophobic solvent comprises petrolatum.

102. The method according to claim 80, wherein the ratio between the hydrophobic solvent and the fatty acid is between about 50:1 and about 20:1, and wherein the ratio between the hydrophobic solvent and the fatty alcohol is between about 30:1 and about 8:1.

103. The method according to claim 80, wherein the dosing schedule is topical application at least once daily for at least 16 days at an effective amount of minocycline hydrochloride of about 4% w/w of the topical composition.

104. The method according to claim 80, wherein the dosing schedule is topical application at least once daily for at least 16 days at an effective amount of minocycline hydrochloride of about 1% to about 6% w/w of the topical composition.

105. A method for treating acne vulgaris in a subject in need thereof, comprising administering to the subject an effective amount of a minocycline antibiotic in a carrier according to a dosing schedule such that
(a) the mean maximum plasma concentration of the tetracycline antibiotic is about 0.2 ng/mL to about 5 ng/mL after at least one day of use, and/or
(b) the mean maximum plasma concentration of the tetracycline antibiotic is about 0.2 ng/mL to about 12 ng/mL after at least 16 days of use, and/or
(c) the area under the plasma concentration versus time curve at the last time point with a detectable drug concentration equal to or greater than the limit of quantification (AUCT) is about 36 ng*h/mL to 132 ng*h/mL,
wherein the carrier is essentially waterless and comprises:
about 48% to about 51% soybean oil by weight of the topical composition,
about 23% to about 25% coconut oil by weight of the topical composition,
about 4% to about 6% cyclomethicone by weight of the topical composition,
about 0.7% to about 2% light mineral oil by weight of the topical composition,
about 3% to about 4% cetostearyl alcohol by weight of the topical composition,
about 2% to about 4% stearic acid by weight of the topical composition,
about 2% to about 3% myristyl alcohol by weight of the topical composition,
about 1% to about 3% hydrogenated castor oil by weight of the topical composition,
about 1% to about 3% beeswax by weight of the topical composition,
about 1% to about 2% stearyl alcohol by weight of the topical composition, and
about 0.5% to about 1.5% behenyl alcohol by weight of the topical composition,
and wherein when the carrier is combined with propellant in an aerosol container to produce a foam.

106. The method according to claim 105, wherein the minocycline antibiotic is minocycline or a salt thereof.

107. The method according to claim 106, wherein the minocycline antibiotic is minocycline hydrochloride.

108. The method according to claim 107, wherein the effective amount of minocycline hydrochloride is about 0.1% w/w to about 10% w/w of the topical composition.

109. The method according to claim 107, wherein the effective amount of minocycline hydrochloride is about 1% w/w to about 6% w/w of the topical composition.

110. The method according to claim 107, wherein the effective amount of minocycline hydrochloride is about 4% w/w of the topical composition.

111. The method according to claim 105, wherein the topical composition is administered for at least 16 days.

112. The method according to claim 105, wherein the propellant is a pressurized gas mixture.

113. The method according to claim 105, wherein the gas mixture is a hydrocarbon gas.

114. The method according to claim 105, further comprising a retinoid.

115. The method according to claim 105, wherein the hydrophobic solvent comprises petrolatum.

116. The method according to claim 105, wherein the ratio between the hydrophobic solvent and the fatty acid is between about 50:1 and about 20:1, and wherein the ratio between the hydrophobic solvent and the fatty alcohol is between about 30:1 and about 8:1.

117. The method according to claim 105, wherein the dosing schedule is topical application at least once daily for at least 16 days at an effective amount of minocycline hydrochloride of about 4% w/w of the topical composition.
